(19) United States
(12) Patent Application Publication
(10) Pub. No.: US 2019/0263934 A1
(43) Pub. Date: Aug. 29, 2019

(54) FC VARIANTS WITH ENHANCED BINDING TO FCRN AND PROLONGED HALF-LIFE

(71) Applicant: GENZYME CORPORATION, Cambridge, MA (US)

(72) Inventors: Huawei Qiu, Westborough, MA (US); Brian Mackness, Bridgewater, NJ (US)

(21) Appl. No.: 16/258,080

(22) Filed: Jan. 25, 2019

Related U.S. Application Data
(60) Provisional application No. 62/622,468, filed on Jan. 26, 2018.

(51) Int. Cl. C07K 16/42

(52) U.S. Cl. CPC C07K 16/4241; C07K 2317/52; A61K 2039/505; C07K 2317/72; C07K 2317/524; C07K 2317/94

(57) ABSTRACT

The present disclosure provides binding polypeptides (e.g., antibodies and immunoadhesins) comprising a modified Fc domain. The present disclosure also provides nucleic acids encoding the binding polypeptides, recombinant expression vectors, and host cells for making such binding polypeptides. Methods of using the binding polypeptides disclosed herein to treat disease are also provided.

### RELATED APPLICATIONS

This application claims priority to U.S. Provisional Patent Application Ser. No. 62/622,468, filed Jan. 26, 2018, the entire disclosure of which is hereby incorporated herein by reference.

### BACKGROUND

The interaction of antibodies with neonatal Fc receptor (FcRn) is a determinant in maintaining and prolonging the serum half-life of antibodies and other Fc-derived therapeutics. FcRn is a heterodimer of an MHC class-I-like α-domain and a β2-macroglobulin (β2-m) subunit which recognizes regions on the antibody Fc heavy chain distinct from other Fcγ receptors (FcγRs) While FcRn is expressed in various tissues, it is thought to act mainly in the vascular endothelium, kidneys and at the blood brain barrier, preventing IgG degradation, excretion and triggering of inflammatory responses, respectively.

Antibody binding to FcRn is highly pH-dependent, and the interaction only occurs with high affinity (high nanomolar to low micromolar) at low pH (pH<6.5), but not at physiological pH (pH approximately 7.4). Upon acidification of the endosome to a pH less than 6.5, the interaction between IgG and FcRn becomes highly favorable, and is directly responsible for inhibiting degradation and promoting recycling of FcRn-bound antibodies to the cell surface. The increase in pH weakens the interaction and promotes release of antibodies into the bloodstream.

Fc engineering using high throughput mutagenesis approaches has been extensively pursued to identify variants that enhance FcRn binding affinity, as enhanced binding would presumably lead to increased efficacy and reduced dosage frequency for therapeutic antibodies as a direct result of a prolonged serum half-life compared to wild-type IgG antibodies. However, variants that enhance FcRn binding affinity can have unpredicted results. For example, certain IgG variants that show large increases in FcRn affinity at pH 6.0, such as N434W or P257I/Q311I among others, have wild-type or severely reduced serum half-lives in cynomolgus monkey and human FcRn (hFcRn) transgenic mouse studies (see, e.g., Kuo et al. 2011 supra; Datta-Mannan et al. 2007, J. Biol. Chem. 282:1709-1717; and Datta-Mannan et al. 2007, Metab. Dispos. 35: 86-94). The T250Q/M428L (QL) variant has shown IgG backbone-specific results in animal models (see, e.g., Datta-Mannan et al. 2007, J. Biol. Chem. 282:1709-1717; and Hinton et al. 2006, J. Immunol. 176:346-356). The M252Y/S254T/T256E (YTE, EU Numbering) variant has shown a 10-fold enhancement in vitro, but displays decreased antibody-dependent cell-mediated cytotoxicity (ADCC) in vivo due to a 2-fold reduction in affinity for the FcγRIIIa receptor (see, e.g., Dall'Acqua et al. 2002 supra).

Thus, there remains a need for alternative Fc variants that possess enhanced binding to FcRn and prolonged circulation half-life.

### SUMMARY

The present invention is based on the discovery of novel IgG antibodies having one or more of the following characteristics: increased serum half-life, enhanced FcRn binding affinity, enhanced FcRn binding affinity at acidic pH, enhanced FcγRIIIa binding affinity, and similar thermal stability, as compared to a wild-type IgG antibody.

Accordingly, in certain aspects, an isolated binding polypeptide comprising a modified Fc domain, comprising an aspartic acid (D) or a glutamic acid (E) at amino acid position 256, and/or a tryptophan (W) or a glutamine (Q) at amino acid position 307, wherein amino acid position 254 is not threonine (T), and further comprising a phenylalanine (F) or a tyrosine (Y) at amino acid position 434, or a tyrosine (Y) at amino acid position 252, wherein the amino acid positions are according to EU numbering, is provided.

In certain exemplary embodiments, the modified Fc domain is a modified human Fc domain. In certain exemplary embodiments, the modified Fc domain is a modified IgG1 Fc domain.

In certain exemplary embodiments, the binding polypeptide has human FcRn binding affinity, rat FcRn binding affinity, or both human and rat FcRn binding affinity.

In certain exemplary embodiments, the isolated binding polypeptide has an altered serum half-life compared to a binding polypeptide comprising a wild-type Fc domain. In certain exemplary embodiments, the isolated binding polypeptide has an increased serum half-life compared to a binding polypeptide comprising a wild-type Fc domain.

In certain exemplary embodiments, the isolated binding polypeptide has altered FcRn binding affinity compared to a binding polypeptide comprising a wild-type Fc domain. In certain exemplary embodiments, the isolated binding polypeptide has enhanced FcRn binding affinity compared to a binding polypeptide comprising a wild-type Fc domain. In certain exemplary embodiments, the isolated binding polypeptide has enhanced FcRn binding affinity at an acidic pH compared to a binding polypeptide comprising a wild-type Fc domain. In certain exemplary embodiments, the isolated binding polypeptide has enhanced FcRn binding affinity at an acidic pH compared to the FcRn binding affinity of the binding polypeptide at an elevated non-acidic pH. In certain exemplary embodiments, an enhanced FcRn binding affinity comprises a reduced FcRn binding off-rate.

In certain exemplary embodiments, an acidic pH is about 6.0. In certain exemplary embodiments, an acidic pH is about 6.0 and a non-acidic pH is about 7.4.

In certain exemplary embodiments, the isolated binding polypeptide has altered FcγRIIIa binding affinity compared to a binding polypeptide comprising a wild-type Fc domain. In certain exemplary embodiments, the isolated binding polypeptide has reduced FcγRIIIa binding affinity compared to a binding polypeptide comprising a wild-type Fc domain. In certain exemplary embodiments, the isolated binding polypeptide has enhanced FcγRIIIa binding affinity compared to a binding polypeptide comprising a wild-type Fc domain.

In certain exemplary embodiments, the isolated binding polypeptide has approximately the same FcγRIIIa binding affinity as a binding polypeptide comprising a wild-type Fc domain.

In certain exemplary embodiments, the isolated binding polypeptide has approximately the same thermal stability as a binding polypeptide comprising a wild-type Fc domain. In certain exemplary embodiments, the isolated binding polypeptide has approximately the same thermal stability as a binding polypeptide comprising a modified Fc domain having the triple amino acid substitution M252Y/S254T/T256E, according to EU numbering.

In certain exemplary embodiments, the isolated binding polypeptide is an antibody, e.g., a monoclonal antibody. In certain exemplary embodiments, the isolated antibody is a chimeric, humanized, or human antibody. In certain exemplary embodiments, the isolated antibody is a full-length antibody.

In certain exemplary embodiments, the isolated binding polypeptide specifically binds one or more human targets.

In other aspects, an isolated binding polypeptide comprising a modified Fc domain comprising a combination of amino acid substitutions at positions selected from the group consisting of a) a tyrosine (Y) at amino acid position 252, and an aspartic acid (D) at amino acid position 256, b) an aspartic acid (D) at amino acid position 256, and a phenylalanine (F) at amino acid position 434, c) an aspartic acid (D) at amino acid position 256, and a tyrosine (Y) at amino acid position 434, d) a tryptophan (W) at amino acid position 307, and a phenylalanine (F) at amino acid position 434, e) a tyrosine (Y) at amino acid position 252, and a tryptophan (W) at amino acid position 307, wherein a tyrosine (Y) is not at amino acid position 434, f) an aspartic acid (D) at amino acid position 256, and a tryptophan (W) at amino acid position 307, wherein a tyrosine (Y) is not at amino acid position 434, g) an aspartic acid (D) at amino acid position 256, and a glutamine (Q) at amino acid position 307, wherein a tyrosine (Y) is not at amino acid position 434, h) a tyrosine (Y) at amino acid position 252, an aspartic acid (D) at amino acid position 256, and a glutamine (Q) at amino acid position 307, wherein a tyrosine (Y) is not at amino acid position 434, and i) a tyrosine (Y) at amino acid position 252, a glutamic acid (E) at amino acid position 256, and a glutamine (Q) at amino acid position 307, wherein a threonine (T) is not at amino acid position 254, a histidine (H) is not at amino acid position 311, and a tyrosine (Y) is not at amino acid position 434, wherein the amino acid substitutions are according to EU numbering, is provided.

In certain exemplary embodiments, the modified Fc domain is a modified human Fc domain. In certain exemplary embodiments, the modified Fc domain is a modified IgG1 Fc domain.

In certain exemplary embodiments, the binding polypeptide has human FcRn binding affinity, rat FcRn binding affinity, or both human and rat FcRn binding affinity.

In certain exemplary embodiments, the isolated binding polypeptide has an altered serum half-life compared to a binding polypeptide comprising a wild-type Fc domain. In certain exemplary embodiments, the isolated binding polypeptide has an increased serum half-life compared to a binding polypeptide comprising a wild-type Fc domain.

In certain exemplary embodiments, the isolated binding polypeptide has altered FcRn binding affinity compared to a binding polypeptide comprising a wild-type Fc domain. In certain exemplary embodiments, the isolated binding polypeptide has enhanced FcRn binding affinity compared to a binding polypeptide comprising a wild-type Fc domain. In certain exemplary embodiments, the isolated binding polypeptide has enhanced FcRn binding affinity at an acidic pH compared to a binding polypeptide comprising a wild-type Fc domain. In certain exemplary embodiments, the isolated binding polypeptide has enhanced FcRn binding affinity at an acidic pH compared to the FcRn binding affinity of the binding polypeptide at an elevated non-acidic pH. In certain exemplary embodiments, an enhanced FcRn binding affinity comprises a reduced FcRn binding off-rate. In certain exemplary embodiments, the isolated binding polypeptide has less FcRn binding affinity at non-acidic pH than a binding polypeptide comprising a modified Fc domain having the double amino acid substitution M428L/N434S, according to EU numbering.

In certain exemplary embodiments, an acidic pH is about 6.0. In certain exemplary embodiments, an acidic pH is about 6.0 and a non-acidic pH is about 7.4.

In certain exemplary embodiments, the isolated binding polypeptide has altered FcγRIIIa binding affinity compared to a binding polypeptide comprising a wild-type Fc domain. In certain exemplary embodiments, the isolated binding polypeptide has reduced FcγRIIIa binding affinity compared to a binding polypeptide comprising a wild-type Fc domain. In certain exemplary embodiments, the isolated binding polypeptide has enhanced FcγRIIIa binding affinity compared to a binding polypeptide comprising a wild-type Fc domain.

In certain exemplary embodiments, the isolated binding polypeptide has approximately the same FcγRIIIa binding affinity as a binding polypeptide comprising a wild-type Fc domain.

In certain exemplary embodiments, the isolated binding polypeptide has approximately the same thermal stability as a binding polypeptide comprising a wild-type Fc domain. In certain exemplary embodiments, the isolated binding polypeptide has approximately the same thermal stability as a binding polypeptide comprising a modified Fc domain having the triple amino acid substitution M252Y/S254T/T256E, according to EU numbering.

In certain exemplary embodiments, the isolated binding polypeptide is an antibody, e.g., a monoclonal antibody. In certain exemplary embodiments, the isolated antibody is a chimeric, humanized, or human antibody. In certain exemplary embodiments, the isolated antibody is a full-length antibody.

In certain exemplary embodiments, the isolated binding polypeptide specifically binds one or more human targets.

In other aspects, an isolated binding polypeptide comprising a modified Fc domain comprising a) a double amino acid substitution selected from the group consisting of M252Y/T256D, M252Y/T256E, M252Y/T307Q, M252Y/T307W, T256D/T307Q, T256D/T307W, T256E/T307Q, and T256E/T307W, wherein a threonine (T) is not at amino acid position 254, a histidine (H) is not at amino acid position 311, and a tyrosine (Y) is not at amino acid position 434, or b) a triple amino acid substitution selected from the group consisting of M252Y/T256D/T307Q, M252Y/T256D/T307W, M252Y/T256E/T307Q, and M252Y/T256E/T307W, wherein a threonine (T) is not at amino acid position 254, a histidine (H) is not at amino acid position 311, and a tyrosine (Y) is not at amino acid position 434, wherein the amino acid substitutions are according to EU numbering, is provided.

In certain exemplary embodiments, the modified Fc domain is a modified human Fc domain. In certain exemplary embodiments, the modified Fc domain is a modified IgG1 Fc domain.

In certain exemplary embodiments, the binding polypeptide has human FcRn binding affinity, rat FcRn binding affinity, or both human and rat FcRn binding affinity.

In certain exemplary embodiments, the isolated binding polypeptide has an altered serum half-life compared to a binding polypeptide comprising a wild-type Fc domain. In certain exemplary embodiments, the isolated binding polypeptide has an increased serum half-life compared to a binding polypeptide comprising a wild-type Fc domain.

In certain exemplary embodiments, the isolated binding polypeptide has altered FcRn binding affinity compared to a binding polypeptide comprising a wild-type Fc domain. In certain exemplary embodiments, the isolated binding polypeptide has enhanced FcRn binding affinity compared to a binding polypeptide comprising a wild-type Fc domain. In certain exemplary embodiments, the isolated binding polypeptide has enhanced FcRn binding affinity at an acidic pH compared to a binding polypeptide comprising a wild-type Fc domain. In certain exemplary embodiments, the isolated binding polypeptide has enhanced FcRn binding affinity at an acidic pH compared to the FcRn binding affinity of the binding polypeptide at an elevated non-acidic pH. In certain exemplary embodiments, an enhanced FcRn binding affinity comprises a reduced FcRn binding off-rate. In certain exemplary embodiments, the isolated binding polypeptide has less FcRn binding affinity at non-acidic pH than a binding polypeptide comprising a modified Fc domain having the double amino acid substitution M428L/N434S, according to EU numbering.

In certain exemplary embodiments, an acidic pH is about 6.0. In certain exemplary embodiments, an acidic pH is about 6.0 and a non-acidic pH is about 7.4.

In certain exemplary embodiments, the isolated binding polypeptide has altered FcγRIIIa binding affinity compared to a binding polypeptide comprising a wild-type Fc domain. In certain exemplary embodiments, the isolated binding polypeptide has reduced FcγRIIIa binding affinity compared to a binding polypeptide comprising a wild-type Fc domain. In certain exemplary embodiments, the isolated binding polypeptide has enhanced FcγRIIIa binding affinity compared to a binding polypeptide comprising a wild-type Fc domain.

In certain exemplary embodiments, the isolated binding polypeptide has approximately the same FcγRIIIa binding affinity as a binding polypeptide comprising a wild-type Fc domain.

In certain exemplary embodiments, the isolated binding polypeptide has approximately the same thermal stability as a binding polypeptide comprising a wild-type Fc domain. In certain exemplary embodiments, the isolated binding polypeptide has approximately the same thermal stability as a binding polypeptide comprising a modified Fc domain having the triple amino acid substitution M252Y/S254T/T256E, according to EU numbering.

In certain exemplary embodiments, the isolated binding polypeptide is an antibody, e.g., a monoclonal antibody. In certain exemplary embodiments, the isolated antibody is a chimeric, humanized, or human antibody. In certain exemplary embodiments, the isolated antibody is a full-length antibody.

In certain exemplary embodiments, the isolated binding polypeptide specifically binds one or more human targets.

In certain aspects, an isolated binding polypeptide comprising a modified Fc domain, wherein the modified Fc domain comprises an aspartic acid (D) at amino acid position 256, and a glutamine (Q) at amino acid position 307, according to EU numbering, is provided.

In certain exemplary embodiments, the modified Fc domain is a modified human Fc domain. In certain exemplary embodiments, the modified Fc domain is a modified IgG1 Fc domain.

In certain exemplary embodiments, the binding polypeptide has human FcRn binding affinity or rat FcRn binding affinity, or both human and rat FcRn binding affinity.

In certain exemplary embodiments, the isolated binding polypeptide has an increased serum half-life compared to a binding polypeptide comprising a wild-type Fc domain.

In certain exemplary embodiments, the isolated binding polypeptide has enhanced FcRn binding affinity compared to a binding polypeptide comprising a wild-type Fc domain. In certain exemplary embodiments, the isolated binding polypeptide has enhanced FcRn binding affinity at an acidic pH compared to a binding polypeptide comprising a wild-type Fc domain. In certain exemplary embodiments, the isolated binding polypeptide has enhanced FcRn binding affinity at an acidic pH compared to the FcRn binding affinity of the binding polypeptide at an elevated non-acidic pH. In certain exemplary embodiments, enhanced FcRn binding affinity comprises a reduced FcRn binding off-rate.

In certain exemplary embodiments, an acidic pH is about 6.0. In certain exemplary embodiments, an acidic pH is about 6.0 and a non-acidic pH is about 7.4.

In certain exemplary embodiments, the isolated binding polypeptide has altered FcγRIIIa binding affinity compared to a binding polypeptide comprising a wild-type Fc domain.

In certain exemplary embodiments, the isolated binding polypeptide is a monoclonal antibody. In certain exemplary embodiments, the antibody is a chimeric, humanized, or human antibody.

In certain exemplary embodiments, the isolated binding polypeptide specifically binds one or more human targets.

In certain aspects, an isolated nucleic acid molecule comprising a nucleic acid encoding the isolated polypeptide, is provided.

In certain aspects, a vector comprising the isolated nucleic acid molecule is provided. In certain exemplary embodiments, the vector is an expression vector. In certain aspects, an expression vector comprising the isolated nucleic acid molecule, is provided.

In certain aspects, a host cell comprising the vector is provided. In certain aspects, a host cell comprising the expression vector, is provided.

In certain exemplary embodiments, the host cell is of eukaryotic or prokaryotic origin. In certain exemplary embodiments, the host cell is of mammalian origin. In certain exemplary embodiments, the host cell is of bacterial origin.

In certain aspects, a pharmaceutical composition comprising the isolated binding polypeptide, is provided.

In certain aspects, a pharmaceutical composition comprising the isolated antibody is provided.

In certain aspects, an isolated binding polypeptide comprising a modified Fc domain, wherein the modified Fc domain comprises an aspartic acid (D) at amino acid position 256, and a tryptophan (W) at amino acid position 307, according to EU numbering, is provided.

In certain exemplary embodiments, the modified Fc domain is a modified human Fc domain. In certain exemplary embodiments, the modified Fc domain is a modified IgG1 Fc domain.

In certain exemplary embodiments, the binding polypeptide has human FcRn binding affinity or rat FcRn binding affinity, or both human and rat FcRn binding affinity.

In certain exemplary embodiments, the isolated binding polypeptide has an increased serum half-life compared to a binding polypeptide comprising a wild-type Fc domain.

In certain exemplary embodiments, the isolated binding polypeptide has enhanced FcRn binding affinity compared to a binding polypeptide comprising a wild-type Fc domain. In certain exemplary embodiments, the isolated binding polypeptide has enhanced FcRn binding affinity at an acidic pH compared to a binding polypeptide comprising a wild-type Fc domain. In certain exemplary embodiments, the isolated binding polypeptide has enhanced FcRn binding affinity at an acidic pH compared to the FcRn binding affinity of the binding polypeptide at an elevated non-acidic pH. In certain exemplary embodiments, enhanced FcRn binding affinity comprises a reduced FcRn binding off-rate.

In certain exemplary embodiments, an acidic pH is about 6.0. In certain exemplary embodiments, an acidic pH is about 6.0 and a non-acidic pH is about 7.4.

In certain exemplary embodiments, the isolated binding polypeptide has altered FcγRIIIa binding affinity compared to a binding polypeptide comprising a wild-type Fc domain.

In certain exemplary embodiments, the isolated binding polypeptide is a monoclonal antibody. In certain exemplary embodiments, the antibody is a chimeric, humanized, or human antibody.

In certain exemplary embodiments, the isolated binding polypeptide specifically binds one or more human targets.

In certain aspects, an isolated nucleic acid molecule comprising a nucleic acid encoding the isolated polypeptide, is provided.

In certain aspects, a vector comprising the isolated nucleic acid molecule is provided. In certain exemplary embodiments, the vector is an expression vector. In certain aspects, an expression vector comprising the isolated nucleic acid molecule, is provided.

In certain aspects, a host cell comprising the vector is provided. In certain aspects, a host cell comprising the expression vector, is provided.

In certain exemplary embodiments, the host cell is of eukaryotic or prokaryotic origin. In certain exemplary embodiments, the host cell is of mammalian origin. In certain exemplary embodiments, the host cell is of bacterial origin.

In certain aspects, a pharmaceutical composition comprising the isolated binding polypeptide, is provided.

In certain aspects, a pharmaceutical composition comprising the isolated antibody is provided.

In certain aspects, an isolated binding polypeptide comprising a modified Fc domain, wherein the modified Fc domain comprises a tyrosine (Y) at amino acid position 252, and an aspartic acid (D) at amino acid position 256, according to EU numbering, is provided.

In certain exemplary embodiments, the modified Fc domain is a modified human Fc domain. In certain exemplary embodiments, the modified Fc domain is a modified IgG1 Fc domain.

In certain exemplary embodiments, the binding polypeptide has human FcRn binding affinity or rat FcRn binding affinity, or both human and rat FcRn binding affinity.

In certain exemplary embodiments, the isolated binding polypeptide has an increased serum half-life compared to a binding polypeptide comprising a wild-type Fc domain.

In certain exemplary embodiments, the isolated binding polypeptide has enhanced FcRn binding affinity compared to a binding polypeptide comprising a wild-type Fc domain. In certain exemplary embodiments, the isolated binding polypeptide has enhanced FcRn binding affinity at an acidic pH compared to a binding polypeptide comprising a wild-type Fc domain. In certain exemplary embodiments, the isolated binding polypeptide has enhanced FcRn binding affinity at an acidic pH compared to the FcRn binding affinity of the binding polypeptide at an elevated non-acidic pH. In certain exemplary embodiments, enhanced FcRn binding affinity comprises a reduced FcRn binding off-rate.

In certain exemplary embodiments, an acidic pH is about 6.0. In certain exemplary embodiments, an acidic pH is about 6.0 and a non-acidic pH is about 7.4.

In certain exemplary embodiments, the isolated binding polypeptide has altered FcγRIIIa binding affinity compared to a binding polypeptide comprising a wild-type Fc domain.

In certain exemplary embodiments, the isolated binding polypeptide is a monoclonal antibody. In certain exemplary embodiments, the antibody is a chimeric, humanized, or human antibody.

In certain exemplary embodiments, the isolated binding polypeptide specifically binds one or more human targets.

In certain aspects, an isolated nucleic acid molecule comprising a nucleic acid encoding the isolated polypeptide, is provided.

In certain aspects, a vector comprising the isolated nucleic acid molecule is provided. In certain exemplary embodiments, the vector is an expression vector. In certain aspects, an expression vector comprising the isolated nucleic acid molecule, is provided.

In certain aspects, a host cell comprising the vector is provided. In certain aspects, a host cell comprising the expression vector, is provided.

In certain exemplary embodiments, the host cell is of eukaryotic or prokaryotic origin. In certain exemplary embodiments, the host cell is of mammalian origin. In certain exemplary embodiments, the host cell is of bacterial origin.

In certain aspects, a pharmaceutical composition comprising the isolated binding polypeptide, is provided.

In certain aspects, a pharmaceutical composition comprising the isolated antibody is provided.

In certain aspects, an isolated binding polypeptide comprising a modified Fc domain, wherein the modified Fc domain comprises a combination of at least four amino acid substitutions comprising: an aspartic acid (D) or a glutamic acid (E) at amino acid position 256, and a tryptophan (W) or a glutamine (Q) at amino acid position 307, wherein amino acid position 254 is not threonine (T), and further comprising: a phenylalanine (F) or a tyrosine (Y) at amino acid position 434; and a tyrosine (Y) at amino acid position 252, wherein amino acid positions are according to EU numbering, is provided.

In certain aspects, an isolated binding polypeptide comprising a modified Fc domain having a combination of amino acid substitutions at positions selected from the group consisting of: a) a tyrosine (Y) at amino acid position 252, an aspartic acid (D) at amino acid position 256, a glutamine (Q) at amino acid position 307, and a tyrosine (Y) at amino acid position 434; b) a tyrosine (Y) at amino acid position 252, a glutamic acid (E) at amino acid position 256, a tryptophan (VV) at amino acid position 307, and a tyrosine (Y) at amino acid position 434; c) a tyrosine (Y) at amino acid position 252, a glutamic acid (E) at amino acid position 256, a glutamine (Q) at amino acid position 307, and a tyrosine (Y) at amino acid position 434; d) a tyrosine (Y) at amino acid position 252, an aspartic acid (D) at amino acid position 256, a glutamine (Q) at amino acid position 307, and a phenylalanine (F) at amino acid position 434; or e) a tyrosine (Y) at amino acid position 252, an aspartic acid (D) at amino acid position 256, a tryptophan (W) at amino acid position 307, and a tyrosine (Y) at amino acid position 434, wherein the amino acid substitutions are according to EU numbering, is provided.

In certain aspects, an isolated binding polypeptide comprising a modified Fc domain comprising: a quadruple amino acid substitution selected from the group consisting of M252Y/T256D/T307Q/N434Y, M252Y/T256E/T307W/N434Y, M252Y/T256E/T307Q/N434Y, M252Y/T256D/T307Q/N434F, and M252Y/T256D/T307W/N434Y, wherein the amino acid substitutions are according to EU numbering, is provided.

In certain exemplary embodiments, the modified Fc domain is a modified human Fc domain. In certain exemplary embodiments, the modified Fc domain is a modified IgG1 Fc domain.

In certain exemplary embodiments, the binding polypeptide has human FcRn binding affinity. In certain exemplary embodiments, the binding polypeptide has rat FcRn binding affinity. In certain exemplary embodiments, the binding polypeptide has human and rat FcRn binding affinity.

In certain exemplary embodiments, the isolated binding polypeptide has altered FcRn binding affinity compared to a binding polypeptide comprising a wild-type Fc domain. In certain exemplary embodiments, the isolated binding polypeptide has enhanced FcRn binding affinity compared to a binding polypeptide comprising a wild-type Fc domain.

In certain exemplary embodiments, the isolated binding polypeptide has enhanced FcRn binding affinity at an acidic pH compared to a binding polypeptide comprising a wild-type Fc domain. In certain exemplary embodiments, the isolated binding polypeptide has enhanced FcRn binding affinity at an acidic pH compared to a binding polypeptide comprising M252Y/S254T/T256E/H433K/N434F.

In certain exemplary embodiments, the isolated binding polypeptide has enhanced FcRn binding affinity at a non-acidic pH compared to a binding polypeptide comprising a wild-type Fc domain. In certain exemplary embodiments, the isolated binding polypeptide has enhanced FcRn binding affinity at a non-acidic pH compared to a binding polypeptide comprising M252Y/S254T/T256E/H433K/N434F.

In certain exemplary embodiments, the isolated binding polypeptide has enhanced FcRn binding affinity at an acidic pH, and enhanced FcRn binding affinity at a non-acidic pH, compared to a binding polypeptide comprising a wild-type Fc domain. In certain exemplary embodiments, the isolated binding polypeptide has enhanced FcRn binding affinity at an acidic pH, and enhanced FcRn binding affinity at a non-acidic pH, compared to a binding polypeptide comprising M252Y/S254T/T256E/H433K/N434F.

In certain exemplary embodiments, the acidic pH is about 6.0. In certain exemplary embodiments, the non-acidic pH is about 7.4.

In certain exemplary embodiments, the isolated binding polypeptide has an altered serum half-life compared to a binding polypeptide comprising a wild-type Fc domain. In certain exemplary embodiments, the isolated binding polypeptide has a reduced serum half-life compared to a binding polypeptide comprising a wild-type Fc domain. In certain exemplary embodiments, the isolated binding polypeptide has a reduced serum half-life compared to a binding polypeptide comprising M252Y/S254T/T256E/H433K/N434F.

In certain exemplary embodiments, the isolated binding polypeptide has altered FcγRIIIa binding affinity compared to a binding polypeptide comprising a wild-type Fc domain. In certain exemplary embodiments, the isolated binding polypeptide has reduced FcγRIIIa binding affinity compared to a binding polypeptide comprising a wild-type Fc domain. In certain exemplary embodiments, the isolated binding polypeptide has reduced FcγRIIIa binding affinity compared to a binding polypeptide comprising M252Y/S254T/T256E/H433K/N434F.

In certain exemplary embodiments, the isolated binding polypeptide has reduced thermal stability compared to a binding polypeptide comprising a wild-type Fc domain. In certain exemplary embodiments, the isolated binding polypeptide has reduced thermal stability compared to a binding polypeptide comprising M252Y/S254T/T256E/H433K/N434F.

In certain exemplary embodiments, the isolated binding polypeptide is an antibody. In certain exemplary embodiments, the isolated binding polypeptide is a monoclonal antibody. In certain exemplary embodiments, the isolated antibody is a chimeric, humanized, or human antibody. In certain exemplary embodiments, the isolated antibody is a full-length antibody.

In certain exemplary embodiments, the isolated binding polypeptide specifically binds one or more targets.

In certain aspects, an isolated nucleic acid molecule comprising a nucleic acid encoding the isolated polypeptide is provided.

In certain aspects, a vector comprising the isolated nucleic acid molecule is provided.

In certain exemplary embodiments, the vector is an expression vector.

In certain aspects, a host cell comprising the vector is provided.

In certain exemplary embodiments, the host cell is of eukaryotic or prokaryotic origin. In certain exemplary embodiments, the host cell is of mammalian origin. In certain exemplary embodiments, the host cell is of bacterial origin.

In certain aspects, a pharmaceutical composition comprising the isolated binding polypeptide is provided.

In certain aspects, a pharmaceutical composition comprising the isolated antibody is provided.

In certain aspects, an isolated binding polypeptide comprising a modified Fc domain comprising a tyrosine (Y) at amino acid position 252, an aspartic acid (D) at amino acid position 256, a glutamine (Q) at amino acid position 307, and a tyrosine (Y) at amino acid position 434, according to EU numbering, is provided.

In certain aspects, an isolated binding polypeptide comprising a modified Fc domain comprising a tyrosine (Y) at amino acid position 252, a glutamic acid (E) at amino acid position 256, a tryptophan (W) at amino acid position 307, and a tyrosine (Y) at amino acid position 434, according to EU numbering, is provided.

In certain aspects, an isolated binding polypeptide comprising a modified Fc domain comprising a tyrosine (Y) at amino acid position 252, a glutamic acid (E) at amino acid position 256, a glutamine (Q) at amino acid position 307, and a tyrosine (Y) at amino acid position 434, according to EU numbering, is provided.

In certain aspects, an isolated binding polypeptide comprising a modified Fc domain comprising a tyrosine (Y) at amino acid position 252, an aspartic acid (D) at amino acid position 256, a glutamine (Q) at amino acid position 307, and a phenylalanine (F) at amino acid position 434, according to EU numbering, is provided.

In certain aspects, an isolated binding polypeptide comprising a modified Fc domain comprising a tyrosine (Y) at amino acid position 252, an aspartic acid (D) at amino acid position 256, a tryptophan (W) at amino acid position 307, and a tyrosine (Y) at amino acid position 434, according to EU numbering, is provided.

In certain exemplary embodiments, the modified Fc domain is a modified human Fc domain. In certain exemplary embodiments, the modified Fc domain is a modified IgG1 Fc domain.

In certain exemplary embodiments, the binding polypeptide has human FcRn binding affinity.

In certain exemplary embodiments, the isolated binding polypeptide has a reduced serum half-life compared to a binding polypeptide comprising a wild-type Fc domain. In certain exemplary embodiments, the isolated binding polypeptide has a reduced serum half-life compared to a binding polypeptide comprising M252Y/S254T/T256E/H433K/N434F.

In certain exemplary embodiments, the isolated binding polypeptide has enhanced FcRn binding affinity at an acidic pH, and enhanced FcRn binding affinity at a non-acidic pH, compared to a binding polypeptide comprising a wild-type Fc domain. In certain exemplary embodiments, the isolated binding polypeptide has enhanced FcRn binding affinity at an acidic pH, and enhanced FcRn binding affinity at a non-acidic pH, compared to a binding polypeptide comprising M252Y/S254T/T256E/H433K/N434F.

In certain exemplary embodiments, the acidic pH is about 6.0 and the non-acidic pH is about 7.4.

In certain exemplary embodiments, the isolated binding polypeptide has reduced FcγRIIIa binding affinity compared to a binding polypeptide comprising a wild-type Fc domain. In certain exemplary embodiments, the isolated binding polypeptide has reduced FcγRIIIa binding affinity compared to a binding polypeptide comprising M252Y/S254T/T256E/H433K/N434F.

In certain exemplary embodiments, the isolated binding polypeptide has reduced thermal stability as a binding polypeptide comprising a wild-type Fc domain. In certain exemplary embodiments, the isolated binding polypeptide has reduced thermal stability compared to a binding polypeptide comprising M252Y/S254T/T256E/H433K/N434F.

In certain exemplary embodiments, the isolated binding polypeptide is a monoclonal antibody. In certain exemplary embodiments, the antibody is a chimeric, humanized, or human antibody.

In certain exemplary embodiments, the isolated binding polypeptide specifically binds one or more targets.

In certain aspects, an isolated nucleic acid molecule comprising a nucleic acid encoding the isolated polypeptide is provided.

In certain aspects, an expression vector comprising the isolated nucleic acid molecule is provided.

In certain aspects, a host cell comprising the expression vector is provided.

In certain aspects, a pharmaceutical composition comprising the isolated binding polypeptide is provided.

In certain aspects, a method of treating a disease or disorder in a subject in need thereof, comprising administering to the subject a therapeutically effective amount of the isolated binding polypeptide, or administering to the subject a therapeutically effective amount of the pharmaceutical composition, is provided.

In certain exemplary embodiments, the disease or disorder is a cancer. In certain exemplary embodiments, the cancer is a tumor.

In certain exemplary embodiments, the disease or disorder is an autoimmune disorder.

In certain aspects, a method of treating a cancer in a subject in need thereof, comprising administering to the subject a therapeutically effective amount of the isolated binding polypeptide, or administering to the subject a therapeutically effective amount of the pharmaceutical composition, is provided.

In certain aspects, a method of treating an autoimmune disorder in a subject in need thereof, comprising administering to the subject a therapeutically effective amount of the isolated binding polypeptide, or administering to the subject a therapeutically effective amount of the pharmaceutical composition, is provided.

### BRIEF DESCRIPTION OF THE DRAWINGS

The foregoing and other features and advantages of the present invention will be more fully understood from the following detailed description of illustrative embodiments taken in conjunction with the accompanying drawings.

FIG. 1A-FIG. 1B depict the structure of an FcRn interacting with an IgG1 Fc region. FIG. 1A depicts an interaction between hFcRn and an IgG1 Fc (pdb: 4n0u) showing one Fc monomer (dark gray ribbon), including the glycosylation shown as sticks labeled by “Glycan,” in complex with the α-domain (gray) and β2-m (light gray) hFcRn subunits. A majority of the antibody residues involved in the interaction with FcRn are located in the loops directly adjacent to the C_{H}2-C_{H}3 interface (dotted line) and opposite the glycosylation site. FIG. 1B depicts a surface representation of the IgG1 Fc crystal structure (pdb: 5d4q) rotated 75° with respect to FIG. 1A. The FcRn binding interface is comprised of residues in the C_{H}2 and C_{H}3 domains. The saturation library was constructed at the eleven positions shown as sticks, as indicated: M252; 1253; S254; T256; K288; T307; K322; E380; L432; N434 and Y436. All of these residues are in close proximity or direct contact with FcRn. The surfaces of the critical histidine residues responsible for the pH dependence (H310, H433, H435) cluster near the positions of interest and are as indicated.

FIG. 2A-FIG. 2D depict an Octet screening assay and results. FIG. 2A schematically presents an Octet screening assay. NiNTA biosensors capture the histidine-tagged antigen and, subsequently, the antibody variants for rat FcRn (rFcRn) binding kinetics. FIG. 2B depicts rFcRn binding kinetic profiles at pH 6.0 of the wild-type (solid), T307A/E380A/N434A (AAA) variant (short dashes), LS (short dashes interspersed by single dot), YTE (long dashes), H435A (long dashes interspersed by single dot) and H310A/H435Q (long dashes interspersed by two dots) antibodies, aligned to the start of the rFcRn association phase. The H435A and H310A/H435Q variants showed little to no FcRn binding. The YTE variant has the slowest FcRn off-rate examined in Octet rFcRn binding assay. FIG. 2C graphically depicts normalization of FcRn binding kinetics at pH 6.0 by a subset of mutants obtained from the Octet screen. Most mutants retained significant binding to rFcRn, but several resembled the mock control (dotted line), indicating the loss of all rFcRn binding (long dashes, located below dotted line (mock)). Two variants (solid lines) had slower rFcRn off-rates than the wild-type antibody (thick long dashes). FIG. 2D depicts a scatterplot analysis of the rFcRn off-rates for all point mutations, with observable rFcRn binding kinetics separated by residue position. The saturation variants fell into one of the following four rFcRn off-rate regimes: no binding (not shown), faster binding (black), wild-type-like binding (white), slower binding (gray). Eighteen mutants showed a significantly slower off-rate from rFcRn than the wild-type antibody (black dashed lines).

FIG. 3 graphically depicts Biacore kinetics of benchmark and wild-type variants with human and rat FcRns at pH 6.0 and pH 7.4. All FcRn binding curves for the concentration series of the wild-type (upper left), AAA variant (upper right), M428/N434S (LS) variant (lower left) and M252Y/S254T/T256E (YTE) variant (lower right) are shown for each human (first and third columns) and rat (second and fourth column) FcRn at pH 6.0 (first and third rows) and pH 7.4 (second and fourth rows). The AAA, LS and YTE variants showed slower off-rates from FcRn than the wild-type antibody. In general, the antibodies bind rFcRn with an approximately 10-fold increased affinity compared to wild-type. The LS variant had the tightest affinity at pH 7.4 and the greatest residual binding at pH 7.4 to hFcRn, while rFcRn bound the YTE variant most tightly.

FIG. 4A graphically depicts Biacore kinetics of the lead saturation variants with human and rat FcRn at pH 6.0. FcRn binding kinetic traces of the concentration series for the 18 lead saturation variants are shown. M252Y, T256D, T256E, N434F, N434P, N434Y, T307A, T307E, T307F, T307Q and T307W had slower off-rates from both human and rat FcRn. The remaining variants were specific for rat FcRn only.

FIG. 4B graphically depicts FcRn binding kinetics of the WT, benchmark and lead single saturation variants with human FcRn at pH 6.0. FcRn binding sensorgrams with a concentration series of the WT, LS, YTE and the 18 saturation variants with human FcRn at pH 6.0. Single saturation variants used for the combination library are underlined and bold.

FIG. 5A-FIG. 5D depict data showing that multiple variants having slower off-rates from both human and rat FcRn at pH 6.0. FIGS. 5A and 5B depict Biacore sensorgrams of various variants. FIG. 5A depicts the off-rates of human FcRn at pH 6.0 for the YTE variant (long dashes interspersed by single dot), LS variant (long dashes interspersed by two dots, wild-type (WT; dotted line), and lead saturation variants (leads; solid lines in various shades). In FIG. 5A, normalized sensorgrams are depicted showing improved hFcRn off-rates compared to the WT.

FIG. 5B depicts the off-rates of rat FcRn at pH 6.0 for the AAA variant (dotted), LS variant (dashes interspersed by two dots), YTE variant (dashes interspersed by single dot), wild-type (solid line) and lead saturation variants (dashed lines in various frequencies and thicknesses). A representative injection of each of the eleven lead antibodies is shown for clarity. These lead single variants showed improved off-rate kinetics from both human and rat FcRn compared to the wild-type. FIG. 5C and FIG. 5D depict binding affinity plots for the lead saturation (white circles) and wild-type (black circle) antibody variants for human (FIG. 5C) and rat (FIG. 5D) FcRn using the on and off-rates obtained from Biacore kinetic measurements. The benchmark variants are shown: AAA (diagonal lines facing bottom right), LS (dotted) and YTE (diagonal lines facing bottom left). Despite the improvement in the FcRn off-rate, a majority of the variants did not have a tighter affinity for human or rat FcRn, due to slower association kinetics. Eleven variants had slower off-rates from both species of FcRn.

FIG. 6A-FIG. 6D depict data showing that combinations of the lead saturation mutations further improved the FcRn off-rates and binding affinities. FIG. 6A and FIG. 6B depict representative Biacore sensorgrams showing FcRn off-rates for human and rat FcRn, respectively. FIG. 6A depicts normalized sensorgrams for human FcRn of a representative variant of the single (dashed line), double (solid light gray line), triple (solid gray line) and quadruple (solid black line) combination variants in comparison to the wild-type (dotted line) and LS variant (long dashes interspersed by two dots). FIG. 6B depicts normalized sensorgrams for rat FcRn of a representative variant of the single (long dashes interspersed by two dots), double (long dashes interspersed by single dot), triple (long dashes), and quadruple (short dashes) combination variants in comparison to the wild-type (dotted line) and YTE variant (solid line). Incorporation of multiple mutations decreased the off-rate and enhanced the binding affinity for FcRn to a greater extent than the benchmark variants. FIG. 6C and FIG. 6D depict plots of combination saturation variants showing on-rate as a function of off-rate for human (FIG. 6C) or rat (FIG. 6D) FcRn, which revealed that a majority of the variants possessed enhanced binding to FcRn at pH 6.0 as compared to the benchmark variants. The tightest binding variants to human and rat FcRn were the quadruple and double combinations, respectively.

FIG. 7A-FIG. 7D depict data showing that enhanced FcRn binding at pH 6.0 disrupted the pH-dependence of the interaction. FIG. 7A and FIG. 7B depict representative sensorgrams of Biacore FcRn binding kinetics at pH 7.4 of the single (long dashes interspersed with two dots), double (long dashes interspersed with single dot), triple (long dashes) and quadruple (short dashes) combination variants in comparison to the wild-type (dotted), and the LS variant (FIG. 7A, solid line) and the YTE variant (FIG. 7B, solid line). Increasing the number of FcRn binding-enhancing mutations resulted in greater residual binding at physiological pH, with most double, triple and quadruple variants showing robust binding to both species of FcRn. FIG. 7C

$$( RU = offset + \frac{( {R}_{max} - offset ) * [ Antibody ]}{[ Antibody ] + {K}_{D , app}} \qquad ( Equation 2 ) )$$

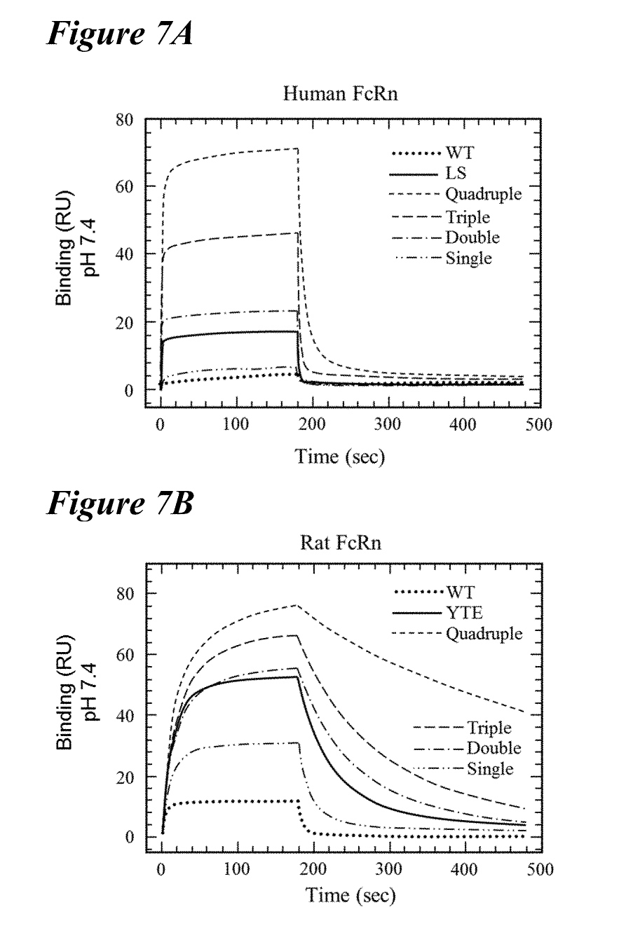

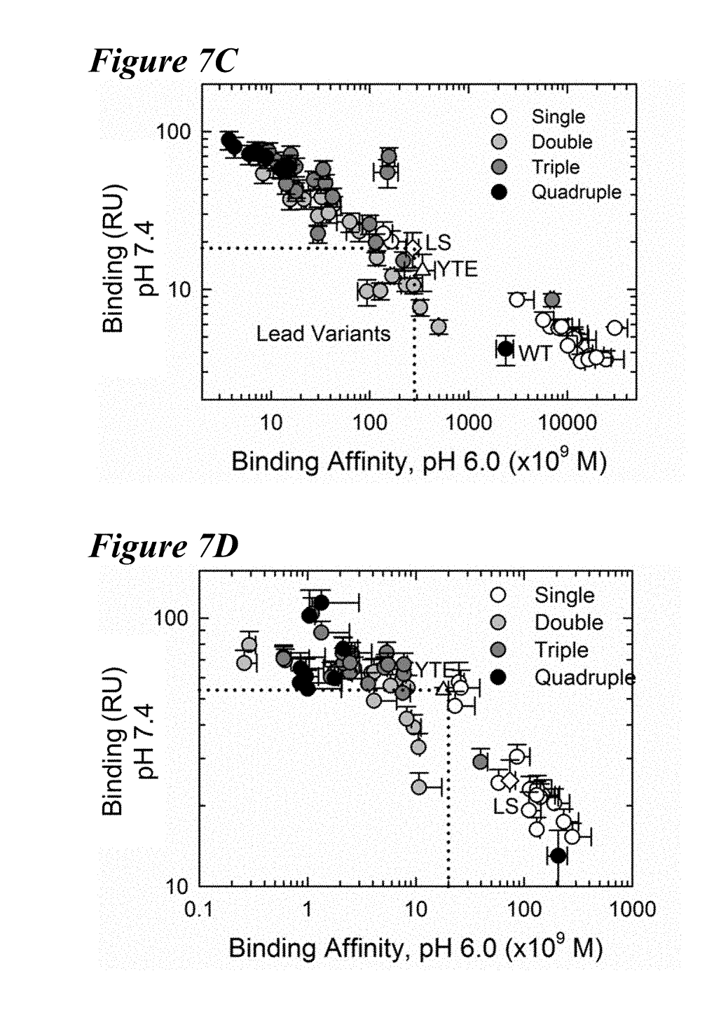

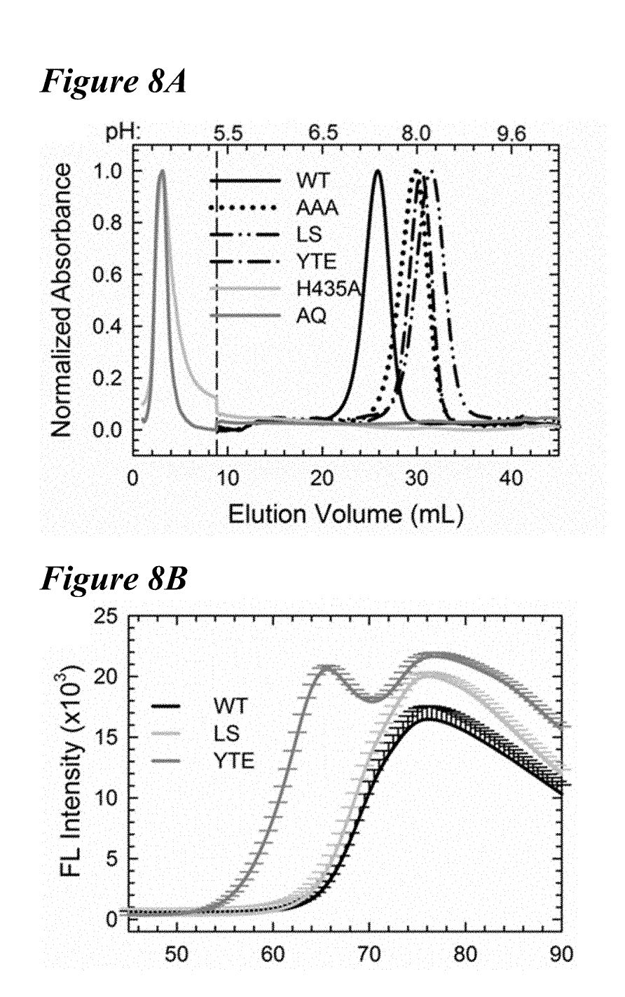

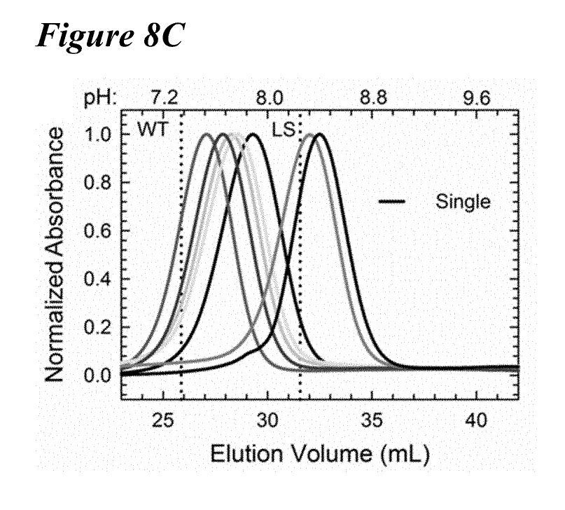

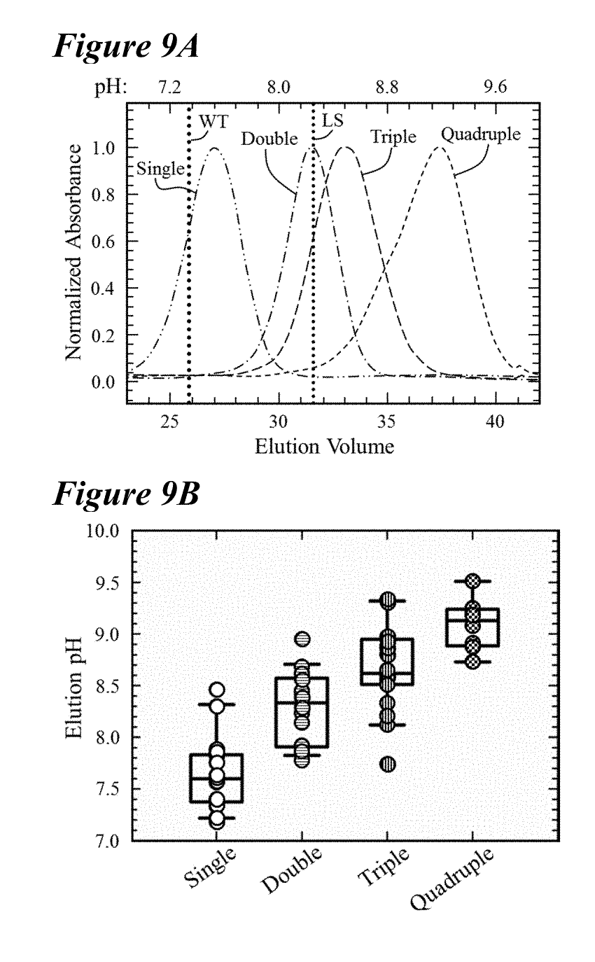

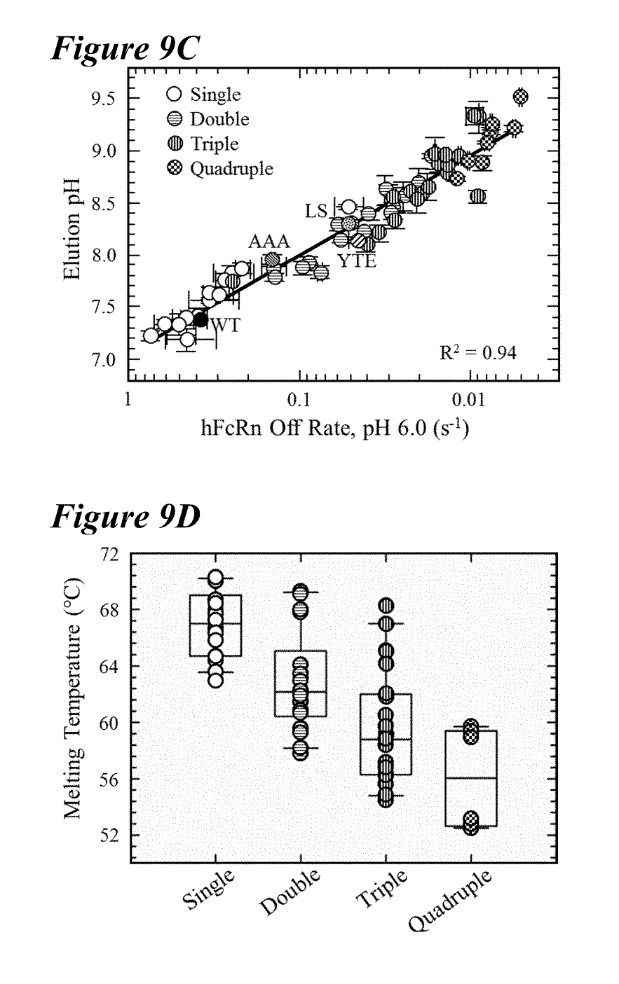

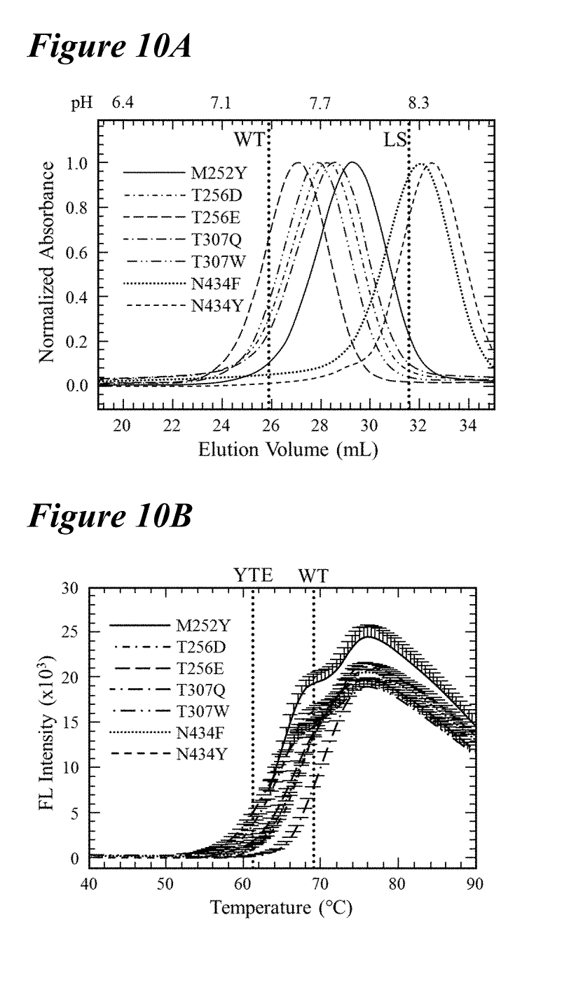

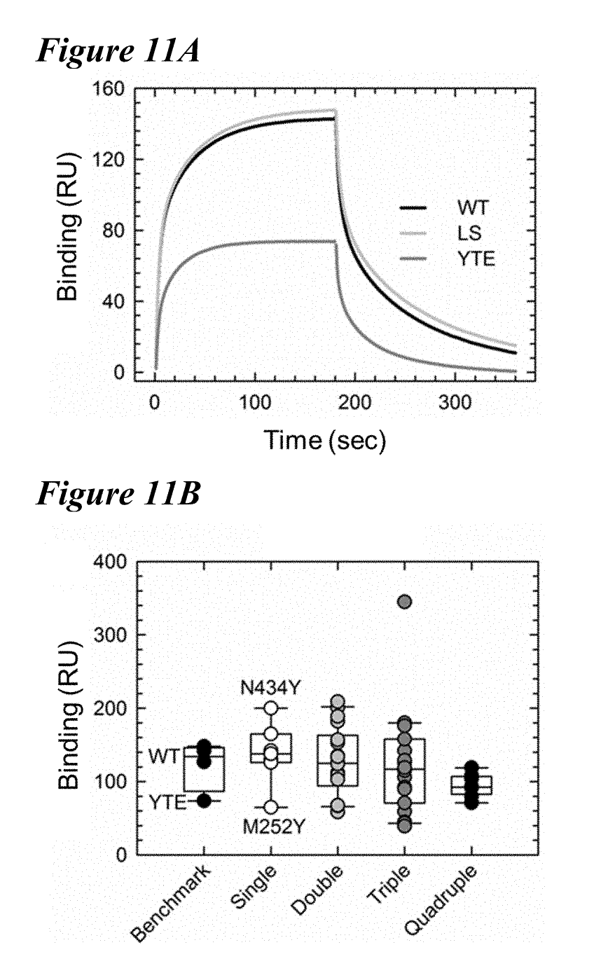

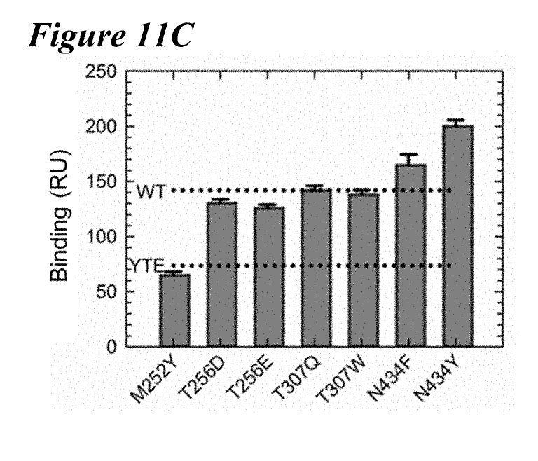

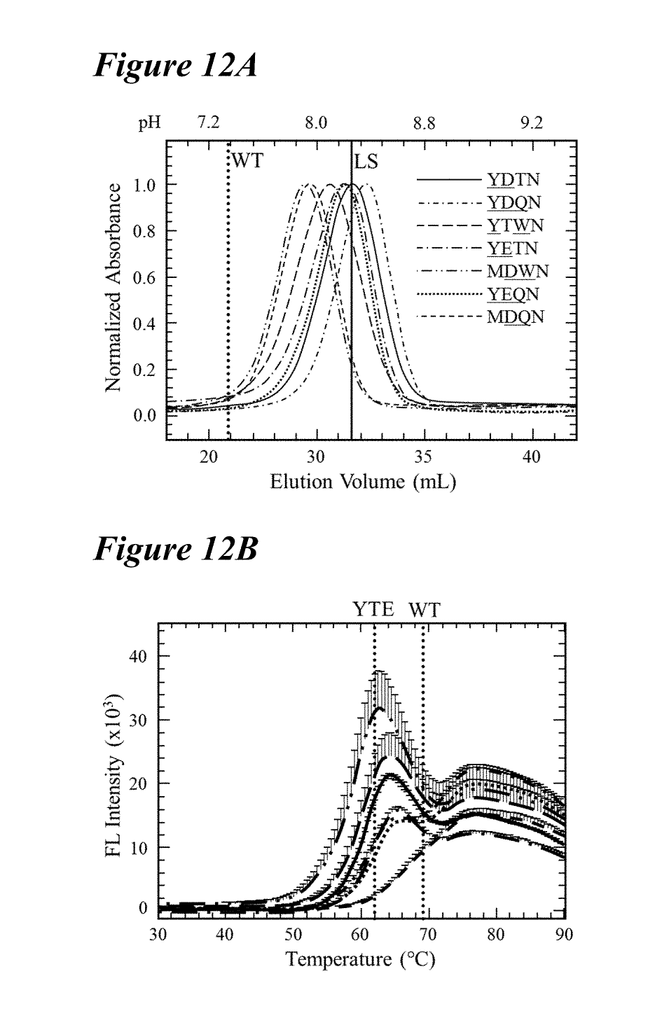

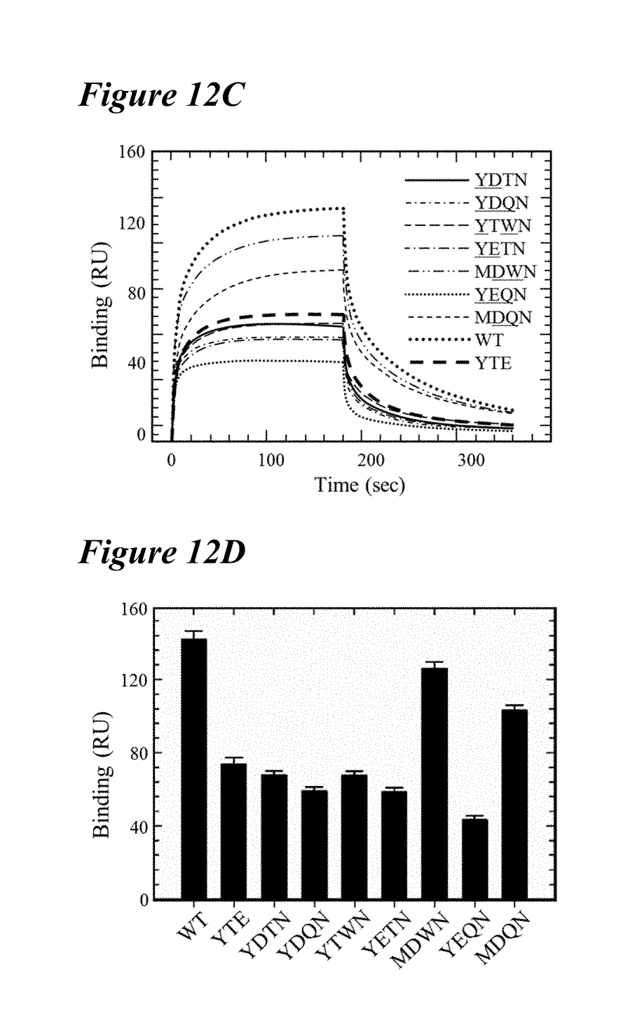

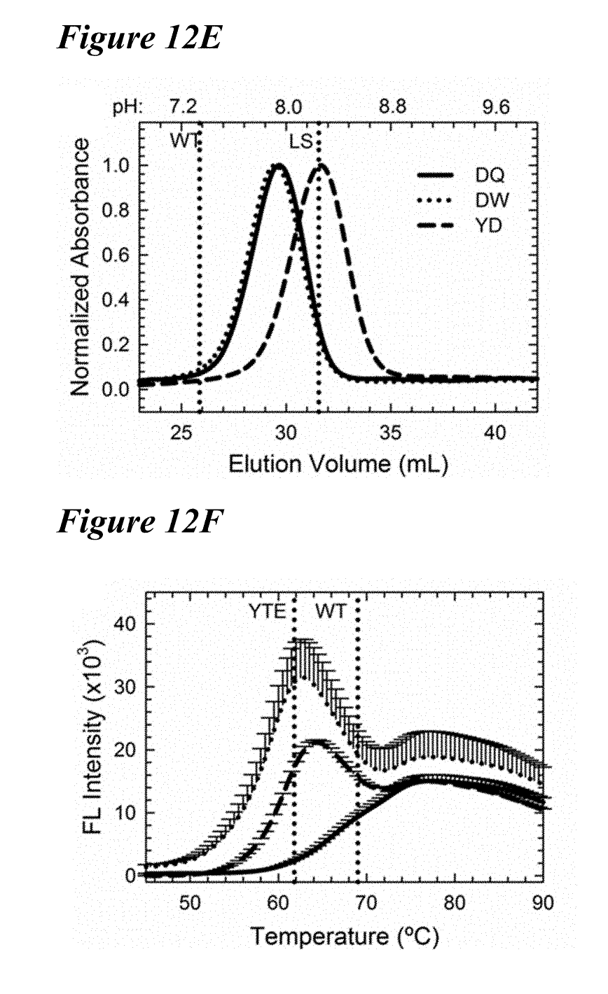

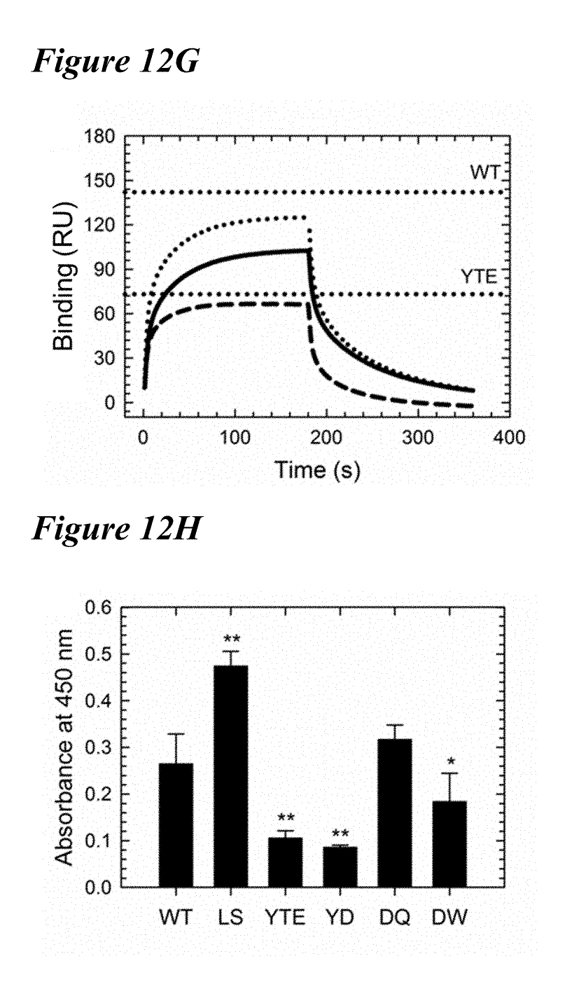

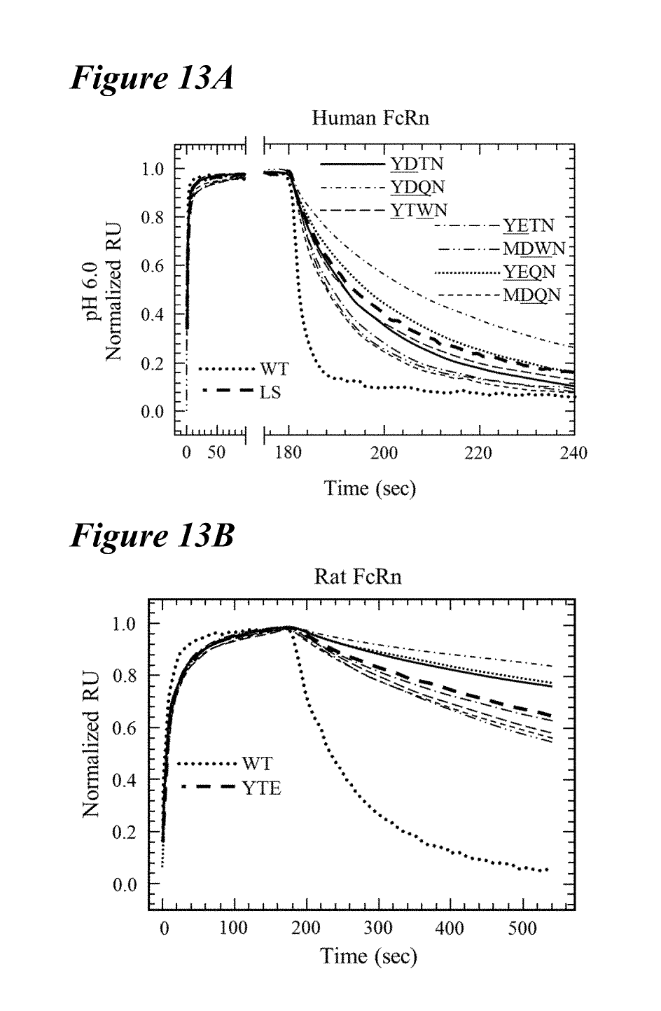

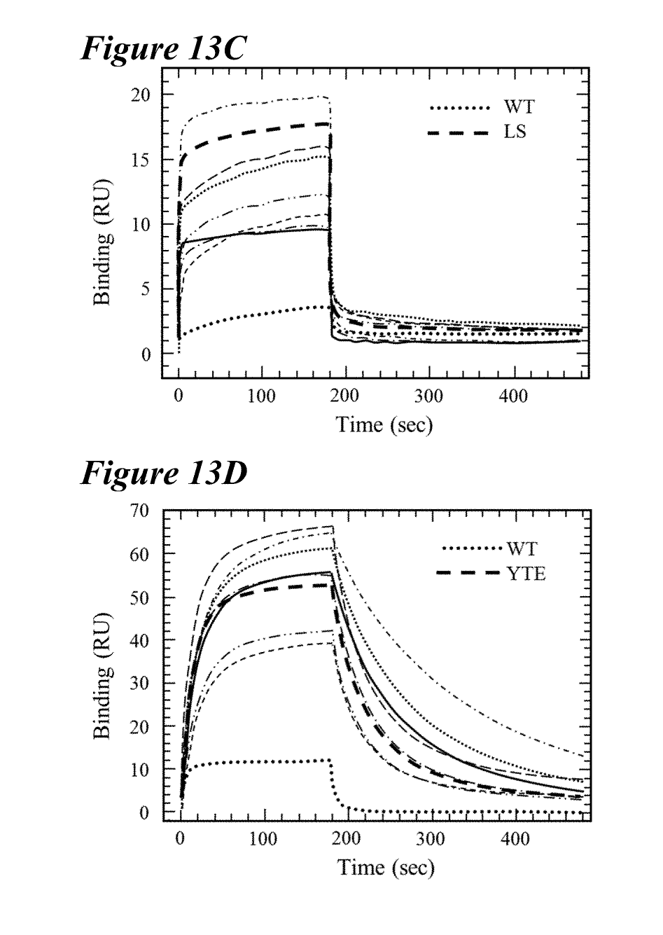

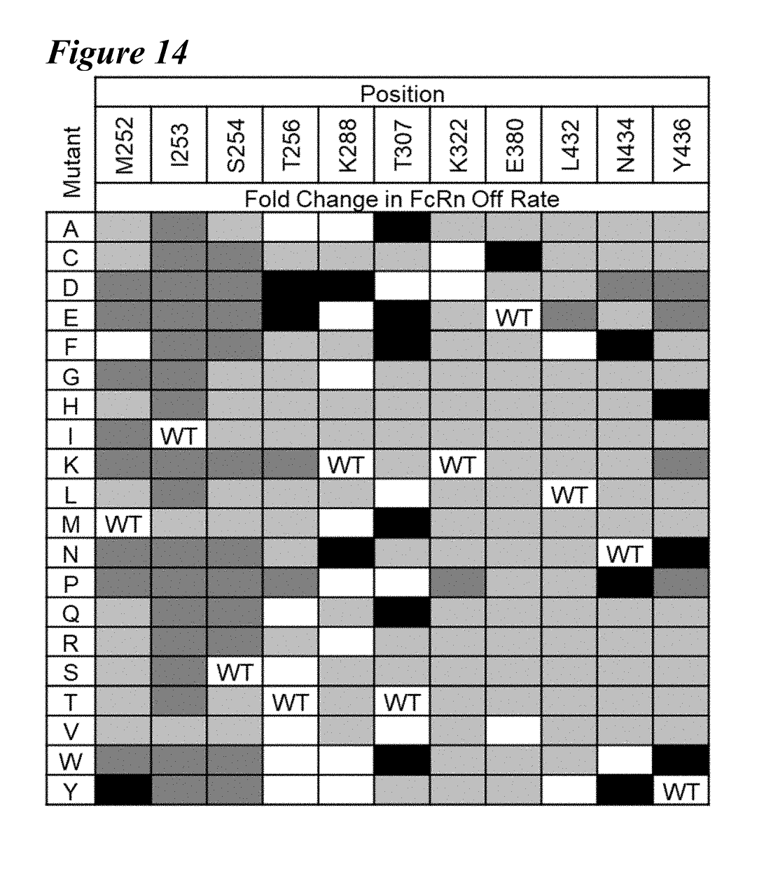

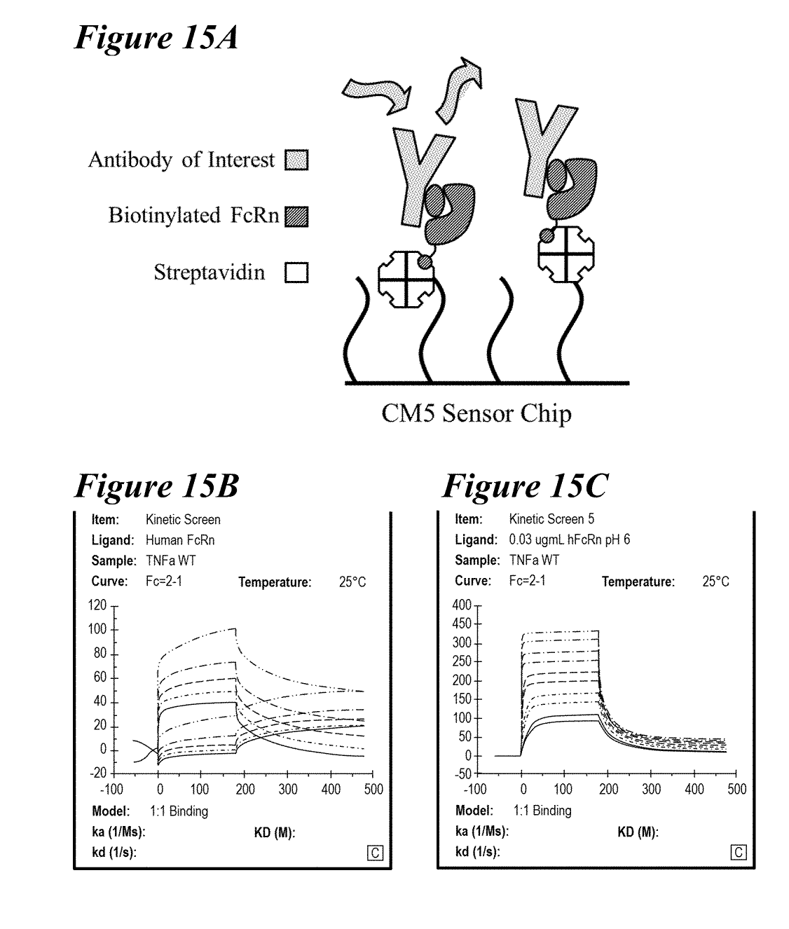

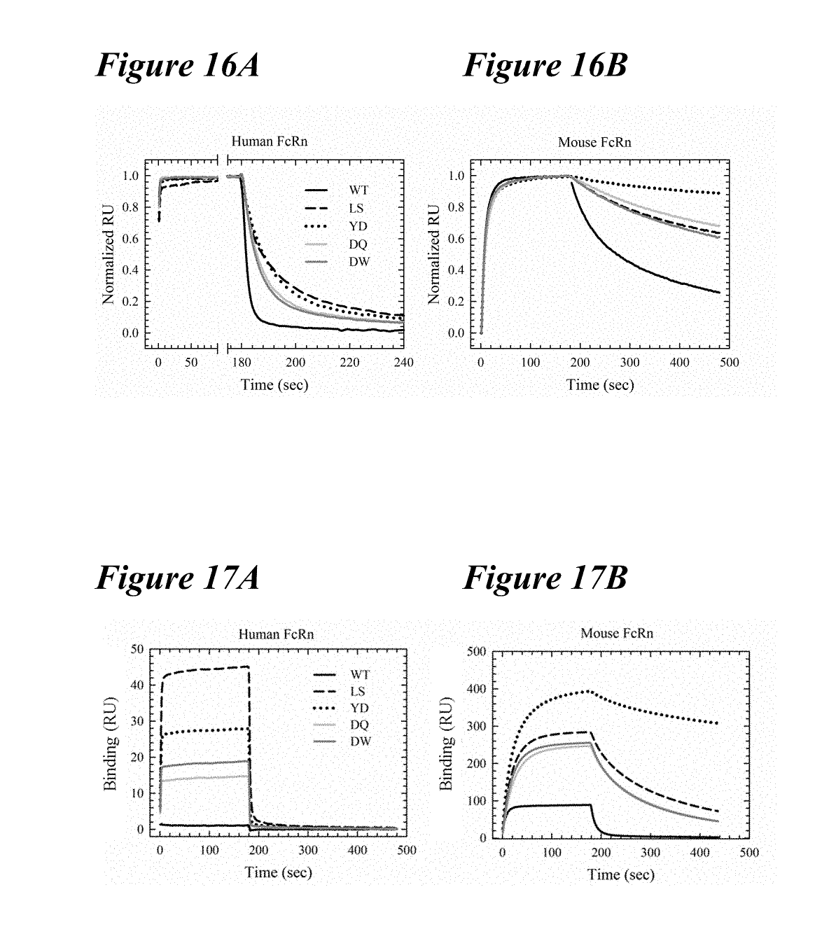

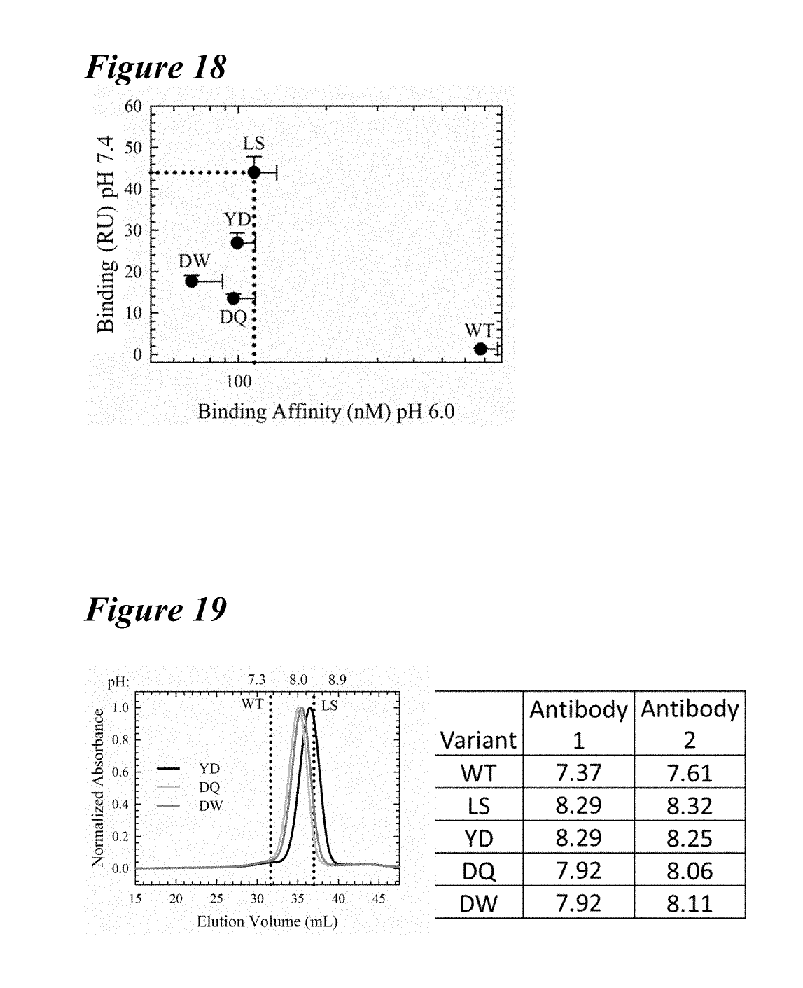

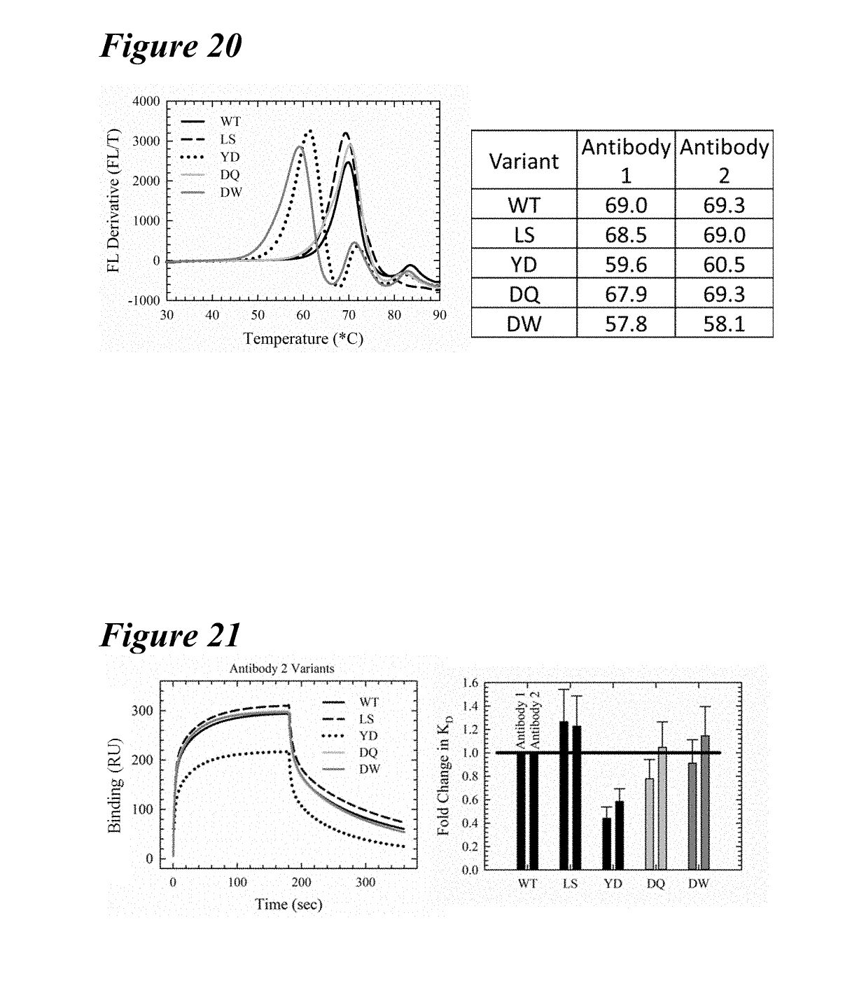

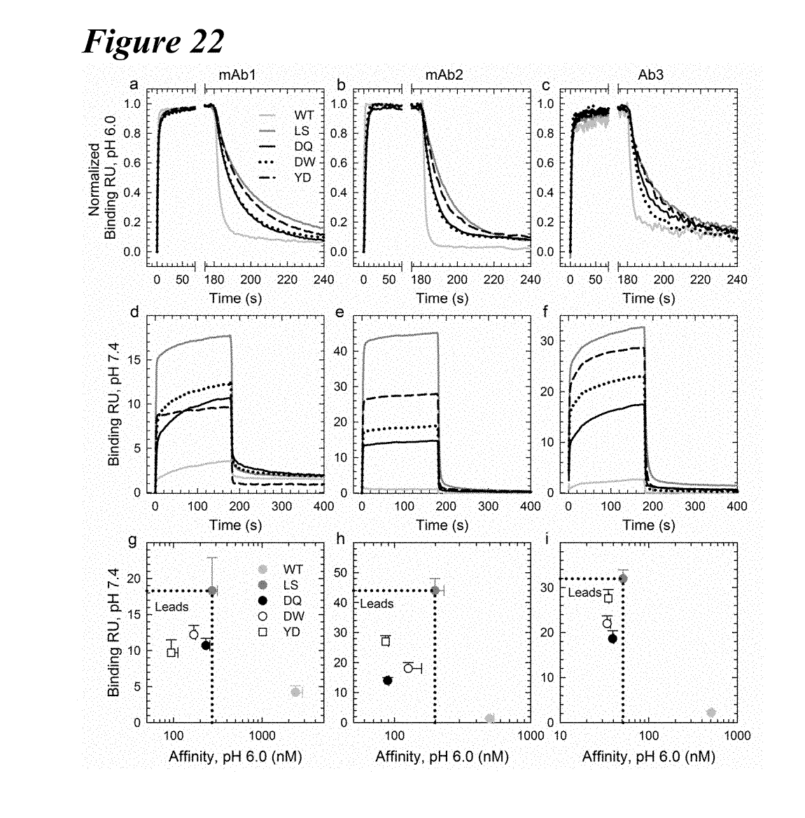

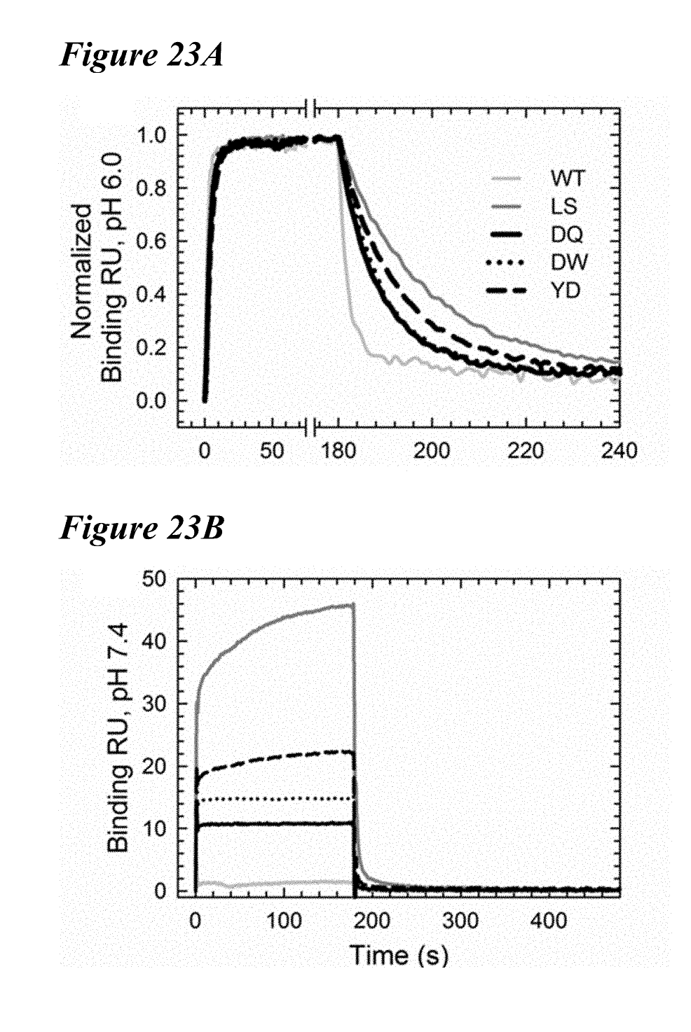

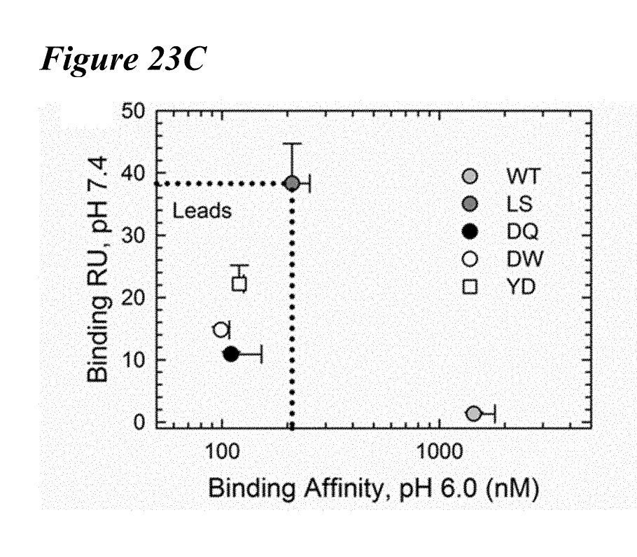

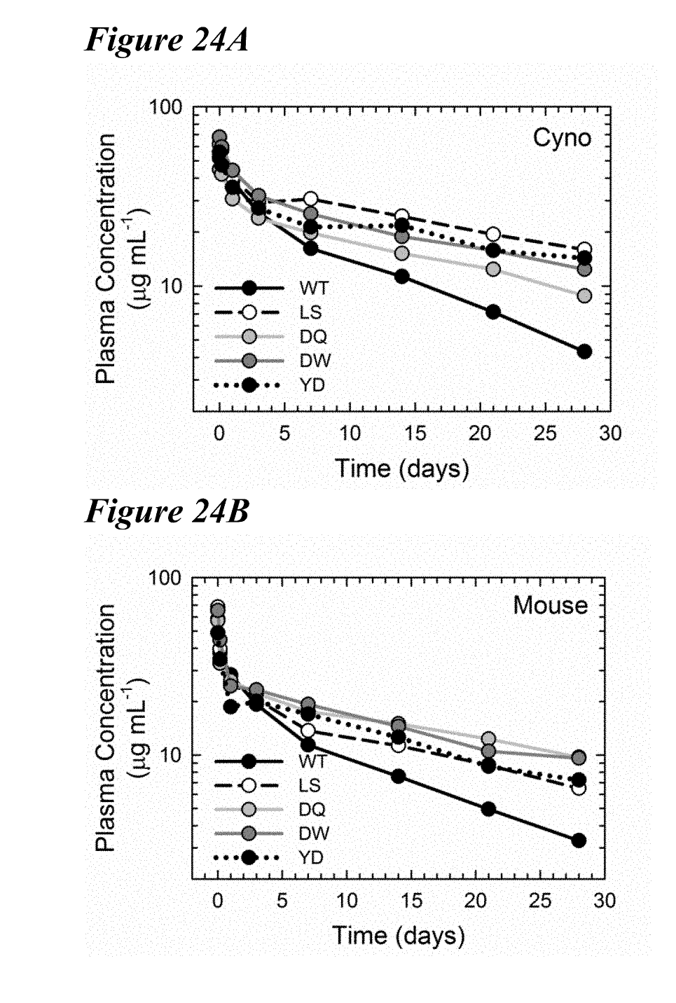

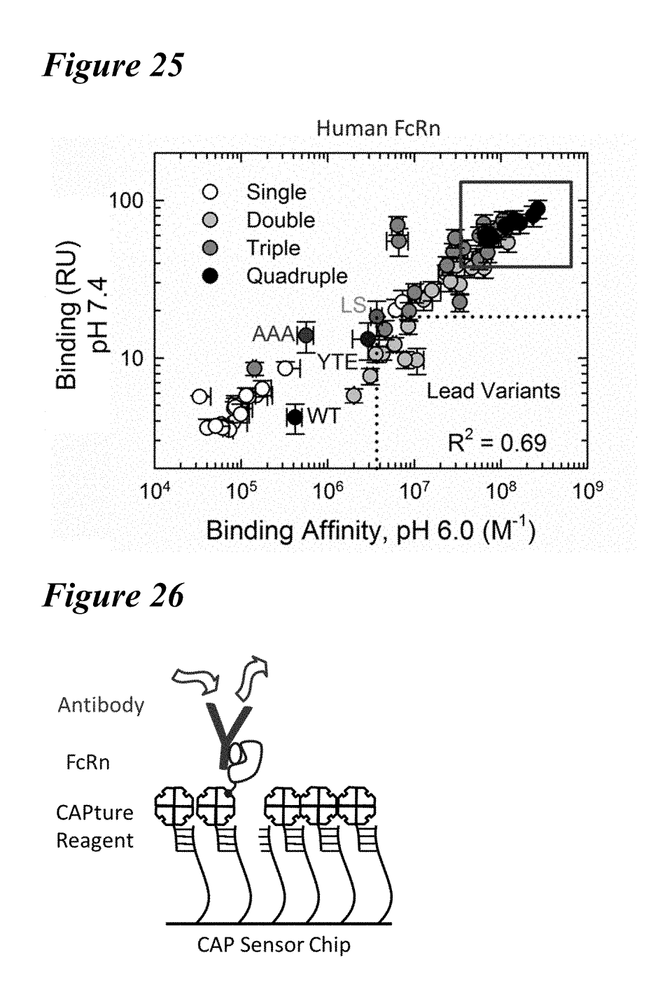

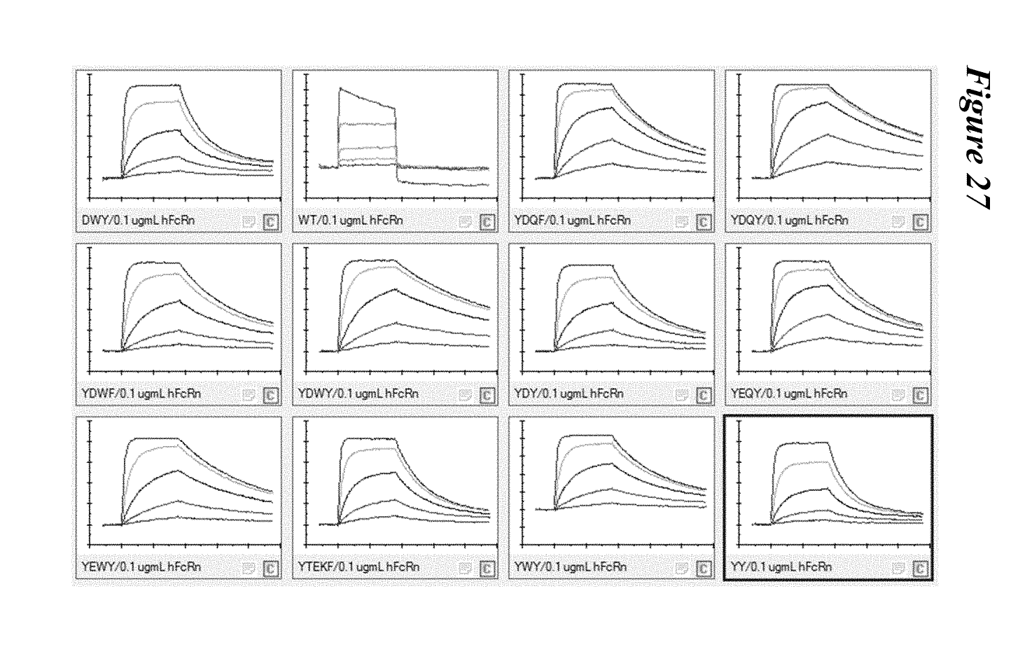

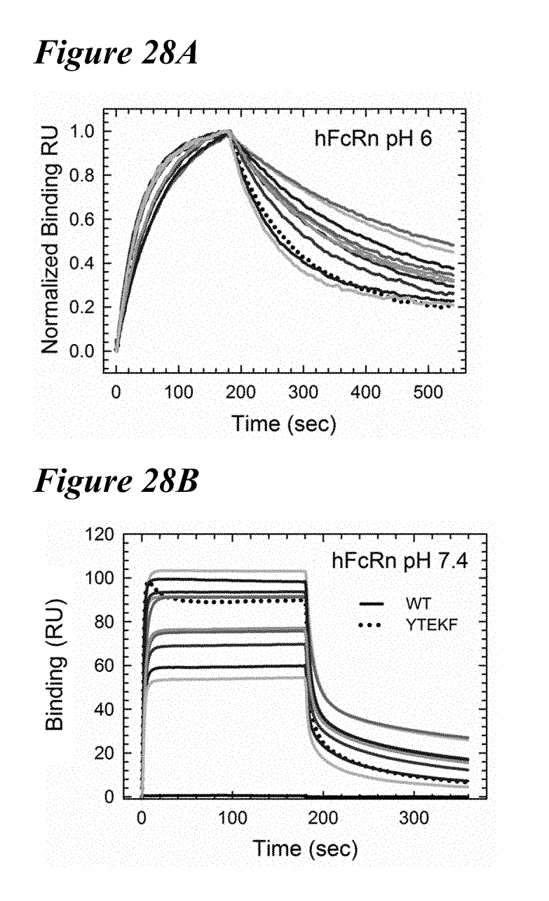

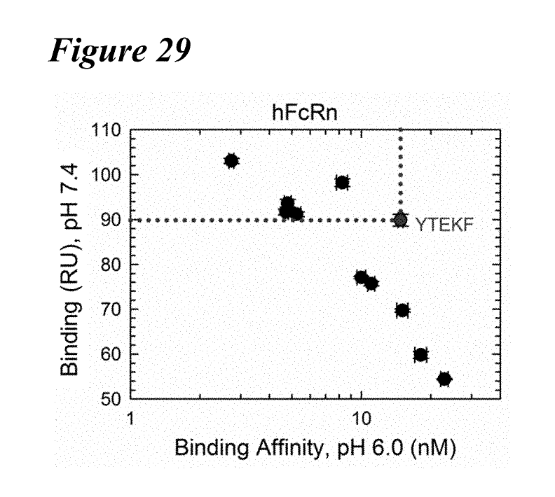

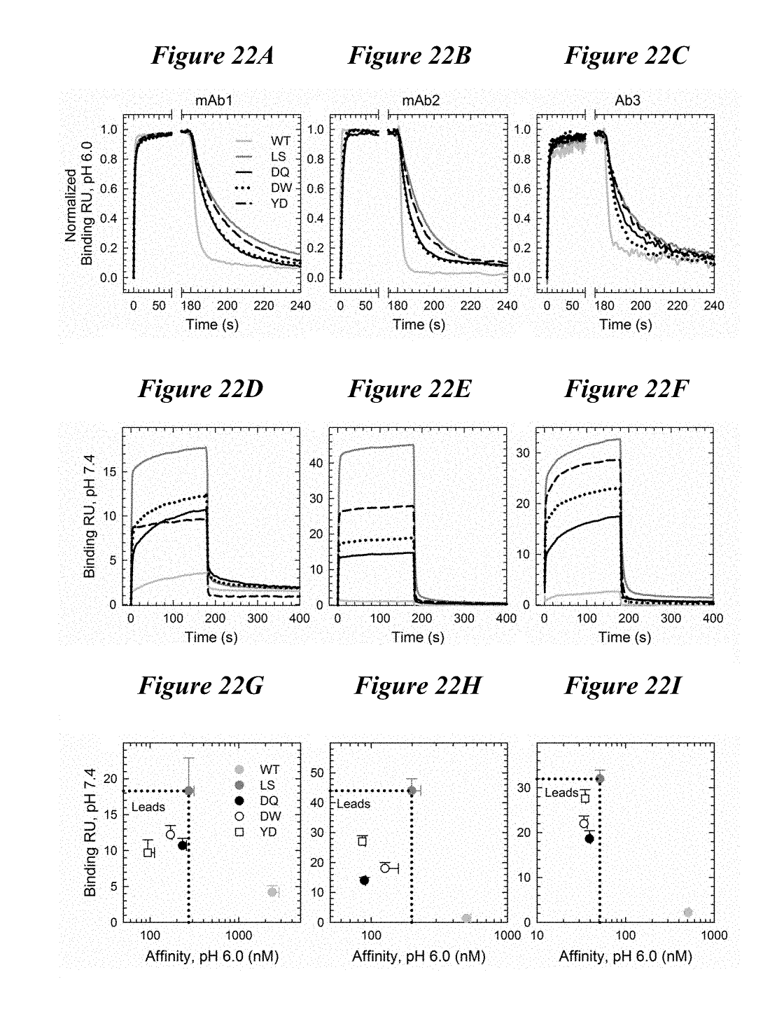

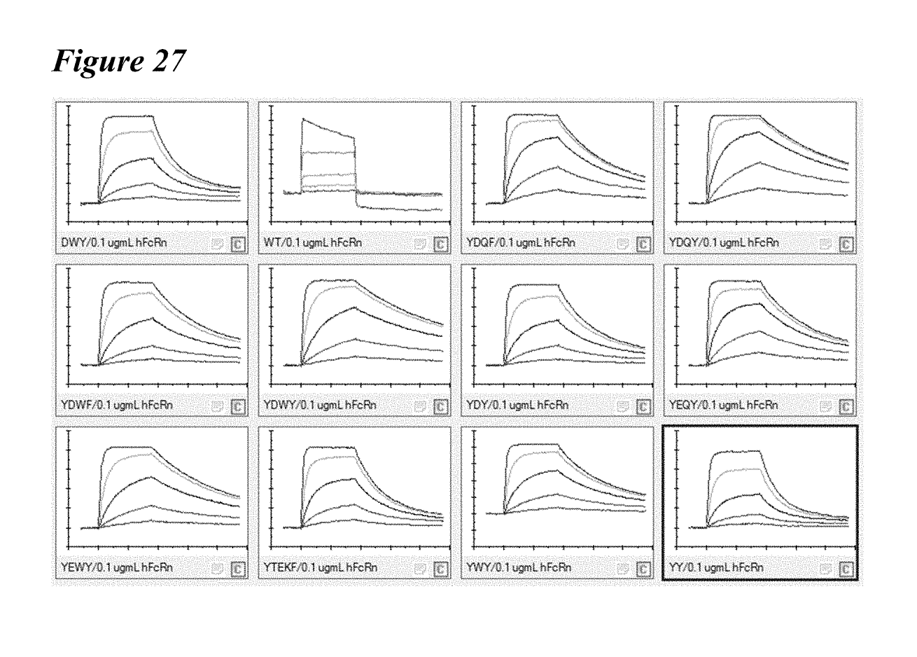

and FIG. 7D depict plots of the steady state RU of all saturation variants to human (FIG. 7C) or rat (FIG. 7D) FcRn at pH 7.4 as a function of the binding affinity at pH 6.0. In FIG. 7C, comparison of the residual FcRn binding at pH 7.4 with the FcRn binding affinity at pH 6.0 is shown. Lead combinations with improved FcRn binding properties occupy the lower left quadrant defined by the LS benchmark variant (diamond). In FIG. 7D, the LS (diamond) and YTE (triangle) variants serve as cutoffs for lead validation, respectively. These two variants had the tightest binding affinity at pH 6.0 and the largest residual binding at pH 7.4 for human and rat FcRn, respectively. In both FIGS. 7C and 7D, single (white circles), double (light gray circles), triple (dark gray circles), and quadruple (black circles) variants as well as the YTE variant (triangle) are shown.

FIG. 8A-FIG. 8C depict data obtained from FcRn affinity chromatography and differential scanning fluorimetry (DSF) of the benchmark variants. FIG. 8A depicts the normalized elution profiles for the WT (solid black line), AAA (dotted line), LS (long dashes interspersed by two dots), YTE (long dashes interspersed by single dot), H435A (solid light gray line) and H310A/H435Q (AQ; solid dark gray line) variants. The pH is noted at the top of the graph. The FcRn binding null variants (H435A, H310A/H435Q) do not bind to the column and elute in the flowthrough (<10 mL). The AAA, LS and YTE variants elute at higher pH than the VVT antibody. FIG. 8B depicts DSF profiles of the WT (black), LS (gray) and YTE (dark gray) variants. YTE was destabilized compared to WT and LS. FIG. 8C depicts FcRn affinity column elution profiles of the seven lead single variants used for the combination variants in comparison to the WT and LS variants (vertical dotted). Two variants (N434F/Y) elute at a higher pH than LS, signifying a reduced pH-dependence on the interaction with FcRn for variants containing these mutations.

FIG. 9A-FIG. 9D depict data showing that combination variants significantly perturbed pH dependence and thermal stability. FIG. 9A depicts representative FcRn affinity chromatograms of single (long dashes interspersed by two dots), double (long dashes interspersed by single dot), triple (long dashes) and quadruple variants (short dashes). Increasing the number of FcRn binding-enhancing mutations shifted the elution towards higher pH values; LS variant (small dotted vertical line). FIG. 9B depicts a box plot of the elution pH for the lead saturation and combination variants, including the single (white circles), double (horizontal lines), triple (vertical lines) and quadruple (checkered) mutants, which indicated a trend toward higher pH values with an increasing number of FcRn enhancing mutants. FIG. 9C shows that the high correlation (R²=0.94) between the elution pH from FcRn affinity chromatography and the hFcRn off-rate using Biacore revealed a loss in the pH-dependence of the antibody-FcRn interaction with improved FcRn dissociation kinetics. The AAA (diagonal lines facing bottom right), LS (dotted) and YTE (diagonal lines facing bottom left) variants had similar hFcRn off-rates and elution pH values as the double variants. FIG. 9D depicts a box plot of the T, obtained from DSF of the combination saturation variants revealed that additional FcRn binding enhancing mutations destabilize the antibody compared to the WT, single or benchmark variants.

FIG. 10A-FIG. 10B depict data obtained from FcRn affinity chromatography and DSF of seven lead variants. FIG. 10A depicts FcRn affinity chromatography of the M252Y (solid line), T256D (short dashes interspersed with single dot), T256E (long dashes), T307Q (long dashes interspersed with single dot), T307W (long dashes interspersed with two dots), N434F (dotted) and N434Y (short dashes) variants. Chromatograms revealed a shift in the elution pH compared to the wild-type and LS antibodies (vertical dotted lines). N434F and N434Y had a higher elution pH (pH approximately 8.3) than the LS variant (vertical dotted line). The pH at certain elution volumes are indicated above the chromatograms for reference. FIG. 10B depicts DSF profiles of seven lead variants, which showed that none of the seven lead single variants destabilized the antibodies to the same extent as the YTE variant (vertical dotted line). All variants, except T307Q (long dashes interspersed with single dot), were destabilized compared to WT (vertical dotted line).

FIG. 11A-FIG. 11C depict data showing that FcγRIIIa binding was reduced in M252Y-containing combination variants. FIG. 11A shows FcγRIIIa binding sensorgrams of the WT (black), LS (gray) and YTE (dark gray) variants revealed a reduced binding response by the YTE variant. FIG. 11B depicts a box plot of the FcγRIIIa binding responses of the benchmark, single and combination variants, as indicated. Variants with the M252Y mutations contain a reduced binding response to FcγRIIIa, including all of the quadruple variants. Combinations with N434F/Y typically show an increased response with FcγRIIIa. FIG. 11C depicts the FcγRIIIa binding responses of the seven lead single variants compared to the WT and YTE variants (horizontal dotted). The M252Y mutation shows a reduced FcγRIIIa binding compared to WT, while six show WT-like or increased binding to this receptor.

FIG. 12A-FIG. 12D depict data obtained from FcRn affinity chromatography, DSF, and FcγRIIIa binding of seven lead combination variants. FIG. 12A depicts FcRn affinity chromatograms of seven lead combination variants in comparison to wild-type antibody and the LS variant (vertical dotted line and solid vertical line respectively). Each lead variant had an elution pH near the LS variant. FIG. 12B shows DSF profiles of the lead combination variants in comparison to the YTE and wild-type variants (vertical dotted lines as indicated). Six of the seven lead variants had a T, that was similar or more destabilized than the YTE variant: MDWN (long dashes interspersed by two dots); YTWN (long dashes); YDTN (solid line); YETN (long dashes interspersed by single dot); YDQN (dotted); YEQN (short dashes interspersed by single dot). The MDQN variant had a similar T, to the wild-type antibody (short dashes). FIG. 12C depicts Biacore sensorgrams of the FcγRIIIa binding kinetics of the seven lead variants in comparison to wild-type (larger dotted line) and the YTE variant (thick long dashes). The M252Y-containing variants, YDTN (solid line), YDQN (short dashes interspersed by single dot), YTWN (long dashes), YETN (long dashes interspersed by single dot) and YEQN (smaller dotted line), each possessed a reduced steady state RU in a similar manner as YTE. (D) shows steady state RU of the seven lead variants, wild-type and YTE variant. Only the MDWN and MDQN variants possessed a similar affinity for FcγRIIIa as the wild-type antibody.

FIG. 12E-FIG. 12H depict data showing that three lead variants displayed a range of key antibody attributes. FIG. 12E shows FcRn affinity chromatography elution profiles of the DQ (solid), DW (dotted) and YD (dashed) variants in comparison to WT and LS (vertical dotted lines). Each double variant showed an elution pH between WT and LS. FIG. 12F depicts DSF fluorescence profiles of the three variants in comparison to the YTE and WT variants (vertical dotted) revealed that YD (dashed) and DW (dotted) were slightly destabilized compared to YTE, but DQ (solid) was similar to the WT. FIG. 12G depicts FcγRIIIa binding sensorgrams in comparison to WT and YTE (horizontal dotted). YD (dashed) showed a similar binding response as YTE, while DQ (solid) and DW (dotted) showed a slight reduction compared to the VVT. FIG. 12H depicts data showing that homogeneous bridging RF ELISA revealed the three lead variants and YTE showed significantly reduced or WT-like RF binding, unlike LS. **p<0.001, *p<0.01.

FIG. 13A-FIG. 13D depict data showing a comparison of FcRn binding kinetics of the lead combination variants at pH 6.0 and pH 7.4. FIG. 13A and FIG. 13B show Biacore FcRn binding sensorgrams of lead combination variants for human FcRn (FIG. 13A) or rat FcRn (FIG. 13B) compared to wild-type (dotted line) and either LS (hFcRn, FIG. 13A, thick long dashes) or YTE (rFcRn, FIG. 13B, thick long dashes) at pH 6.0. Each combination variant had an overall tighter binding affinity to the respective FcRn despite altered on- and off-rates. FIG. 13C and FIG. 13D show Biacore FcRn sensorgrams at pH 7.4. Each hFcRn lead variant had a similar or reduced steady state FcRn binding response as compared to the LS variant. Only the MDQN and MDWN variants showed less rFcRn binding at pH 7.4 than the YTE variant.

FIG. 14 is a table depicting Octet rFcRn Binding Off-rates of a Saturation Library according to certain embodiments. Wild-type (WT) and wild-type-like (WT-like) species are indicated by white rectangles; WT species are as indicated. Variants with little to no rFcRn binding compared to wildtype are indicated by dark gray rectangles. Variants with faster rFcRn off-rate as compared to wildtype are indicated by light gray rectangles, and variants with slower rFcRn off-rate as compared to wildtype are indicated by black rectangles.

FIG. 15A-FIG. 15C depict a new binding assay developed using a CM5 sensor chip. FIG. 15A is a schematic of the assay. FIG. 15B shows direct immobilization of FcRn. FIG. 15C shows streptavidin capture of biotinylated FcRn.

FIG. 16A-FIG. 16B depict FcRn binding of Antibody-2 at pH 6.0. FIG. 16A depicts human FcRn. FIG. 16B depicts mouse FcRn.

FIG. 17A-FIG. 17B depict FcRn binding of Antibody-2 at pH 7.4. FIG. 17A depicts human FcRn. FIG. 17B depicts mouse FcRn.

FIG. 18 graphically depicts the pH-dependence of various Antibody-2 variants. Lead variants maintained a higher binding affinity at pH 6 and a lower residual binding at pH 7.4 than LS.

FIG. 19 depicts a comparison of FcRn binding pH dependence using the backbones of Antibody-1 and Antibody-2.

FIG. 20 depicts a comparison of thermal stability using the backbones of Antibody-1 and Antibody-2.

FIG. 21 depicts a comparison of FcγRIIIa binding using the backbones of Antibody-1 and Antibody-2.

FIG. 22A-FIG. 221 depict multiple plots showing that the DQ, DW and YD variants were transferable among IgG1 backbones. Plots a-c depict normalized FcRn binding sensorgrams at pH 6.0 in three IgG1 backbones with the WT (light gray), LS (dark gray), DQ (solid black), DW (dotted) and YD (dashed) variants showing similar kinetics at low pH. These three variants, DQ, DW and YD, possessed slightly faster on and off-rates than the LS variant but maintained a tighter FcRn binding affinity. Plots d-f depict FcRn binding sensorgrams at pH 7.4; LS benchmark variant (solid black). Plots g-i depict the FcRn binding response at pH 7.4 compared to the binding affinity at pH 6.0 for each antibody backbone with the WT (gray), LS (dark gray), DQ (solid black), DW (empty) and YD (empty square) variants. DQ, DW and YD show improved FcRn characteristics, with enhanced binding at pH 6.0 and minimal binding at pH 7.4.

FIG. 23A-FIG. 23C show that the three lead variants in the mAb2 backbone similarly improves the binding to cynomolgus FcRn. FIG. 23A depicts normalized cFcRn binding sensorgrams at pH 6.0 of WT (gray), LS (dark gray), DQ (solid black), DW (dotted) and YD (dashed) showing similar binding kinetics and affinities as hFcRn. FIG. 23B depicts that the cFcRn binding response for the three variants was dramatically reduced at physiological pH; LS (dark gray), but showed greater binding than WT (gray) in a similar manner as hFcRn. FIG. 23C depicts a comparison of the residual cFcRn binding response at pH 7.4 with the cFcRn binding affinity at pH 6.0 of WT (gray), LS (dark gray), DQ (solid black), DW (empty) and YD (empty square), revealing all three variants maintained the improved FcRn binding properties observed with hFcRn.

FIG. 24A-FIG. 24B show that the lead variants prolonged the antibody serum half-life. Pharmacokinetic profiles of the plasma antibody concentration as a function of time in cynomolgus monkey (FIG. 24A) and hFcRn transgenic mouse (FIG. 24B) of the WT (black circles with solid black line), LS (white circles with dashed black line), DQ (light gray circles with solid light gray line), DW (dark gray circles with solid dark gray line) and YD (black circles with dotted black line) antibodies. All three lead variants prolong the antibody half-life compared to the WT.

FIG. 25 depicts a plot of the steady state RU of all saturation variants to human FcRn at pH 7.4 as a function of the binding affinity at pH 6.0. Comparison of the residual FcRn binding at pH 7.4 with the FcRn binding affinity at pH 6.0 is shown. Quadruple combinations with improved FcRn binding properties at both pH 6.0 and pH 7.4 are shown boxed in upper right quadrant of plot. Single (white circles), double (light gray circles), triple (dark gray circles), and quadruple (black circles) variants as well as the benchmark AAA, LS, and YTE variants (as indicated) are shown.

FIG. 26 depicts a schematic of the Biotin CAPture method used to capture biotinylated FcRn.

FIG. 27 depicts plots showing human FcRn binding kinetics at pH 6.0 of the YTEKF benchmark and combination variants as indicated.

FIG. 28A-FIG. 28B show the FcRn binding kinetics of the combination variants in comparison to the YTEKF benchmark at pH 6.0 (FIG. 28A) and at pH 7.4 (FIG. 28B). Wild-type is indicated by a solid black line (WT) and the YTEKF benchmark is indicated by a dotted line.

FIG. 29 depicts a plot of the steady state RU of select variants to human FcRn at pH 7.4 as a function of the binding affinity at pH 6.0 compared to the YTEKF benchmark. Several variants (lead quadruple variants) exhibited enhanced binding affinity to human FcRn at pH 6.0 and pH 7.4 over the YTEKF benchmark.

### DETAILED DESCRIPTION

The present disclosure provides binding polypeptides (e.g., antibodies) having altered Fc neonatal receptor (FcRn) binding affinities. In certain embodiments, the binding polypeptides comprise a modified Fc domain that enhances FcRn binding affinity compared to a binding polypeptide that comprises a wild-type (e.g., non-modified) Fc domain. The present disclosure also provides nucleic acids encoding binding polypeptides, recombinant expression vectors and host cells for making binding polypeptides, and pharmaceutical compositions comprising the binding polypeptides disclosed herein. Methods of using the binding polypeptides of the present disclosure to treat diseases are also provided.

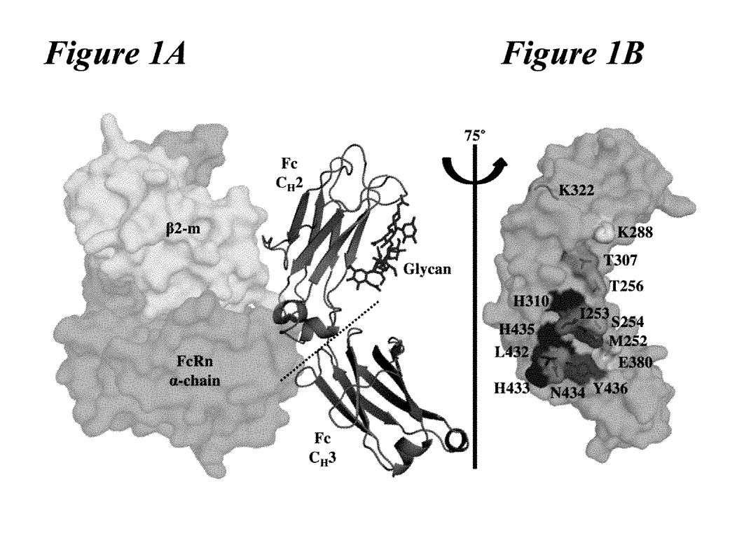

Fc domains of immunoglobulins are involved in non-antigen binding functions and have several effector functions mediated by binding of effector molecules, e.g., binding of the FcRn. As illustrated in FIG. 1A, Fc domains are comprised of a CH2 domain and a CH3 domain. A majority of the residues involved in the interaction with FcRn are located in the loops directly adjacent to the C_{H}2-C_{H}3 interface (FIG. 1A, dotted line) and opposite the glycosylation site. FIG. 1B illustrates the surface representation of the IgG1 Fc crystal structure (pdb: 5d4q) and shows residues in the CH2 and CH3 domains that comprise the FcRn binding interface. The present disclosure provides binding polypeptides comprising a modified Fc domain. Binding polypeptides comprising a modified Fc domain can be antibodies, or immunoadhesins, or Fc fusion proteins.

In certain embodiments, a binding polypeptide may comprise a modified Fc domain comprising an amino acid substitution which alters the antigen-independent effector functions of the antibody, in particular, the circulating half-life (e.g., serum half-life) of the binding polypeptide. In some embodiments, a binding polypeptide may comprise a modified Fc domain comprising an amino acid substitution which alters the serum half-life of the binding polypeptide, compared to a binding polypeptide comprising a wild-type (i.e., non-modified) Fc domain. In some embodiments, a binding polypeptide may comprise a modified Fc domain comprising an amino acid substitution which increases the serum half-life of the binding polypeptide, compared to a binding polypeptide comprising a wild-type (i.e., non-modified) Fc domain. In some embodiments, a binding polypeptide may comprise a modified Fc domain comprising an amino acid substitution which decreases the serum half-life of the binding polypeptide, compared to a binding polypeptide comprising a wild-type (i.e., non-modified) Fc domain.

In certain embodiments, a binding polypeptide that comprises a modified Fc domain that alters (i.e., increases or decreases) the circulating half-life (e.g., the serum half-life) further contains one or more mutations in addition to the mutation(s) that alter the circulating half-life. In certain embodiments, the one or more mutations in addition to the mutation(s) that alter the circulating half-life provide one or more desired biochemical characteristics such as, e.g., one or more of reduced or enhanced effector functions, the ability to non-covalently dimerize, increased ability to localize at the site of a tumor, reduced serum half-life, increased serum half-life when compared with a whole, unaltered antibody of approximately the same immunogenicity and the like.

Binding polypeptides described herein may exhibit either increased or decreased binding to the neonatal Fc receptor (FcRn) when compared to binding polypeptides lacking these substitutions, and therefore, have an increased or decreased serum half-life, respectively. Fc domains with improved affinity for FcRn are expected to have longer serum half-lives, and such molecules have useful applications in methods of treating mammals where long half-life of the administered antibody is desired, e.g., to treat a chronic disease or disorder. In contrast, Fc domains with decreased FcRn binding affinity are expected to have shorter serum half-lives, and such molecules are also useful, for example, for administration to a mammal where a shortened circulation time may be advantageous, e.g., for in vivo diagnostic imaging or in situations where the starting antibody has toxic side effects when present in the circulation for prolonged periods. Fc domains with decreased FcRn binding affinity are also less likely to cross the placenta and, thus, are also useful in the treatment of diseases or disorders in pregnant women. In addition, other applications in which reduced FcRn binding affinity may be desired include applications localized to the brain, kidney, and/or liver.

It is to be understood that the methods described in this disclosure are not limited to particular methods and experimental conditions disclosed herein as such methods and conditions may vary. It is also to be understood that the terminology used herein is for the purpose of describing particular embodiments only, and is not intended to be limiting.

Furthermore, the experiments described herein, unless otherwise indicated, use conventional molecular and cellular biological and immunological techniques within the skill of the art. Such techniques are well known to the skilled worker, and are explained fully in the literature. See, e.g., Ausubel, et al., ed., Current Protocols in Molecular Biology, John Wley & Sons, Inc., NY, N.Y. (1987-2008), including all supplements, aMolecular Cloning: A Laboratory Manual (Fourth Edition) by M R Green and J. Sambrook and Harlow et al., Antibodies: A Laboratory Manual, Chapter 14, Cold Spring Harbor Laboratory, Cold Spring Harbor (2013, 2^{nd} edition).

Unless otherwise defined, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

Generally, nomenclature used in connection with cell and tissue culture, molecular biology, immunology, microbiology, genetics and protein and nucleic acid chemistry and hybridization described herein is well-known and commonly used in the art. The methods and techniques provided herein are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

That the disclosure may be more readily understood, select terms are defined below.

The term “polypeptide” refers to any polymeric chain of amino acids and encompasses native or artificial proteins, polypeptide analogs or variants of a protein sequence, or fragments thereof, unless otherwise contradicted by context. A polypeptide may be monomeric or polymeric. A polypeptide fragment comprises at least about 5 contiguous amino acids, at least about 10 contiguous amino acids, at least about 15 contiguous amino acids, or at least about 20 contiguous amino acids, for example.

The term “isolated protein” or “isolated polypeptide” refers to a protein or polypeptide that by virtue of its origin or source of derivation is not associated with naturally associated components that accompany it in its native state; is substantially free of other proteins from the same species; is expressed by a cell from a different species; or does not occur in nature. Thus, a protein or polypeptide that is chemically synthesized or synthesized in a cellular system different from the cell from which it naturally originates will be “isolated” from its naturally associated components. A protein or polypeptide may also be rendered substantially free of naturally associated components by isolation using protein purification techniques well known in the art.

As used herein, the term “binding protein” or “binding polypeptide” shall refer to a protein or polypeptide (e.g., an antibody or immunoadhesin) that contains at least one binding site which is responsible for selectively binding to a target antigen of interest (e.g., a human target antigen). Exemplary binding sites include an antibody variable domain, a ligand binding site of a receptor, or a receptor binding site of a ligand. In certain aspects, the binding proteins or binding polypeptides comprise multiple (e.g., two, three, four, or more) binding sites. In certain aspects, the binding protein or binding polypeptide is not a therapeutic enzyme.

The term “ligand” refers to any substance capable of binding, or of being bound, to another substance. Similarly, the term “antigen” refers to any substance to which an antibody may be generated. Although “antigen” is commonly used in reference to an antibody binding substrate, and “ligand” is often used when referring to receptor binding substrates, these terms are not distinguishing, one from the other, and encompass a wide range of overlapping chemical entities. For the avoidance of doubt, antigen and ligand are used interchangeably throughout herein. Antigens/ligands may be a peptide, a polypeptide, a protein, an aptamer, a polysaccharide, a sugar molecule, a carbohydrate, a lipid, an oligonucleotide, a polynucleotide, a synthetic molecule, an inorganic molecule, an organic molecule, and any combination thereof.

The term “specifically binds” as used herein, refers to the ability of an antibody or an immunoadhesin to bind to an antigen with a dissociation constant (Kd) of at most about 1×10⁻⁶ M, about 1×10⁻⁷ M, about 1×10⁻⁸ M, about 1×10⁻⁹ M, about 1×10⁻¹⁰ M, about 1×10⁻¹¹ M, about 1×10⁻¹² M or less, and/or to bind to an antigen with an affinity that is at least about two-fold greater than its affinity for a nonspecific antigen.

As used herein, the term “antibody” refers to such assemblies (e.g., intact antibody molecules, immunoadhesins, or variants thereof) which have significant known specific immunoreactive activity to an antigen of interest (e.g. a tumor associated antigen). Antibodies and immunoglobulins comprise light and heavy chains, with or without an interchain covalent linkage between them. Basic immunoglobulin structures in vertebrate systems are relatively well understood.

As will be discussed in more detail below, the generic term “antibody” comprises five distinct classes of antibody that can be distinguished biochemically. While all five classes of antibodies are clearly within the scope of the current disclosure, the following discussion will generally be directed to the IgG class of immunoglobulin molecules. With regard to IgG, immunoglobulins comprise two identical light chains of molecular weight approximately 23,000 Daltons, and two identical heavy chains of molecular weight 53,000-70,000. The four chains are joined by disulfide bonds in a “Y” configuration wherein the light chains bracket the heavy chains starting at the mouth of the “Y” and continuing through the variable region.

Light chains of immunoglobulin are classified as either kappa (κ) or lambda (λ). Each heavy chain class may be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells, or genetically engineered host cells. In the heavy chain, the amino acid sequences run from an N-terminus at the forked ends of the Y configuration to the C-terminus at the bottom of each chain. Those skilled in the art will appreciate that heavy chains are classified as gamma (γ), mu (μ), alpha (α), delta (δ), or epsilon (ε), with some subclasses among them (e.g., γ1-γ4). It is the nature of this chain that determines the “class” of the antibody as IgG, IgM, IgA IgG, or IgE, respectively. The immunoglobulin isotype subclasses (e.g., IgG1, IgG2, IgG3, IgG4, IgA1, etc.) are well-characterized and are known to confer functional specialization. Modified versions of each of these classes and isotypes are readily discernable to the skilled artisan in view of the instant disclosure and, accordingly, are within the scope of the current disclosure.

Both the light and heavy chains are divided into regions of structural and functional homology. The term “region” refers to a part or portion of an immunoglobulin or antibody chain and includes constant region or variable regions, as well as more discrete parts or portions of said regions. For example, light chain variable regions include “complementarity determining regions” or “CDRs” interspersed among “framework regions” or “FRs,” as defined herein.

The regions of an immunoglobulin heavy or light chain may be defined as “constant” (C) region or “variable” (V) regions, based on a relative lack of sequence variation within the regions of various class members in the case of a “constant region,” or based on a significant variation within the regions of various class members in the case of a “variable regions.” The terms “constant region” and “variable region” may also be used functionally. In this regard, it will be appreciated that the variable regions of an immunoglobulin or antibody determine antigen recognition and specificity. Conversely, the constant regions of an immunoglobulin or antibody confer important effector functions such as secretion, trans-placental mobility, Fc receptor binding, complement binding, and the like. The subunit structures and three-dimensional configurations of the constant regions of the various immunoglobulin classes are well-known.

The constant and variable regions of immunoglobulin heavy and light chains are folded into domains. The term “domain” refers to a globular region of a heavy or light chain comprising peptide loops (e.g., comprising 3 to 4 peptide loops) stabilized, for example, by β-pleated sheet and/or an intra-chain disulfide bond. Constant region domains on the light chain of an immunoglobulin are referred to interchangeably as “light chain constant region domains,” “CL regions” or “CL domains.” Constant domains on the heavy chain (e.g., hinge, CH1, CH2 or CH3 domains) are referred to interchangeably as “heavy chain constant region domains,” “CH” region domains or “CH domains.” Variable domains on the light chain are referred to interchangeably as “light chain variable region domains,” “VL region domains” or “VL domains.” Variable domains on the heavy chain are referred to interchangeably as “heavy chain variable region domains,” “VH region domains” or “VH domains.”

By convention, the numbering of the amino acids of the variable constant region domains increases as they become more distal from the antigen-binding site or amino-terminus of the immunoglobulin or antibody. The N-terminus of each heavy and light immunoglobulin chain is a variable region and the C-terminus is a constant region. The CH3 and CL domains comprise the carboxy-terminus of the heavy and light chain, respectively. Accordingly, the domains of a light chain immunoglobulin are arranged in a VL-CL orientation, while the domains of the heavy chain are arranged in the VH-CH1-hinge-CH2-CH3 orientation.

The assignment of amino acids to each variable region domain is in accordance with the definitions of Kabat, Sequences of Proteins of Immunological Interest (National Institutes of Health, Bethesda, Md., 1987 and 1991). Kabat also provides a widely used numbering convention (Kabat numbering) in which corresponding residues between different heavy chain variable regions or between different light chain variable regions are assigned the same number. CDRs 1, 2 and 3 of a VL domain are also referred to herein, respectively, as CDR-L1, CDR-L2 and CDR-L3. CDRs 1, 2 and 3 of a VH domain are also referred to herein, respectively, as CDR-H1, CDR-H2 and CDR-H3. If so noted, the assignment of CDRs can be in accordance with IMGT® (Lefranc et al., Developmental & Comparative Immunology 27:55-77; 2003) in lieu of Kabat. Numbering of the heavy chain constant region is via the EU index as set forth in Kabat (Kabat, Sequences of Proteins of Immunological Interest, National Institutes of Health, Bethesda, Md., 1987 and 1991).

As used herein, the term “VH domain” includes the amino terminal variable domain of an immunoglobulin heavy chain, and the term “VL domain” includes the amino terminal variable domain of an immunoglobulin light chain.

As used herein, the term “CH1 domain” includes the first (most amino terminal) constant region domain of an immunoglobulin heavy chain that extends, e.g., from about positions 114-223 in the Kabat numbering system (EU positions 118-215). The CH1 domain is adjacent to the VH domain and amino terminal to the hinge region of an immunoglobulin heavy chain molecule, and does not form a part of the Fc region of an immunoglobulin heavy chain.

As used herein, the term “hinge region” includes the portion of a heavy chain molecule that joins the CH1 domain to the CH2 domain. The hinge region comprises approximately 25 residues and is flexible, thus allowing the two N-terminal antigen binding regions to move independently. Hinge regions can be subdivided into three distinct domains: upper, middle, and lower hinge domains (Roux et al. J. Immunol. 1998, 161:4083).

As used herein, the term “CH2 domain” includes the portion of a heavy chain immunoglobulin molecule that extends, e.g., from about positions 244-360 in the Kabat numbering system (EU positions 231-340). The CH2 domain is unique in that it is not closely paired with another domain. Rather, two N-linked branched carbohydrate chains are interposed between the two CH2 domains of an intact native IgG molecule. In one embodiment, a binding polypeptide of the current disclosure comprises a CH2 domain derived from an IgG1 molecule (e.g. a human IgG1 molecule).

As used herein, the term “CH3 domain” includes the portion of a heavy chain immunoglobulin molecule that extends approximately 110 residues from N-terminus of the CH2 domain, e.g., from about positions 361-476 of the Kabat numbering system (EU positions 341-445). The CH3 domain typically forms the C-terminal portion of the antibody. In some immunoglobulins, however, additional domains may extend from the CH3 domain to form the C-terminal portion of the molecule (e.g., the CH4 domain in the μ chain of IgM and the e chain of IgE). In one embodiment, a binding polypeptide of the current disclosure comprises a CH3 domain derived from an IgG1 molecule (e.g., a human IgG1 molecule).

As used herein, the term “CL domain” includes the constant region domain of an immunoglobulin light chain that extends, e.g., from about Kabat position 107A to about Kabat position 216. The CL domain is adjacent to the VL domain. In one embodiment, a binding polypeptide of the current disclosure comprises a CL domain derived from a kappa light chain (e.g., a human kappa light chain).

As used herein, the term “Fc region” is defined as the portion of a heavy chain constant region beginning in the hinge region just upstream of the papain cleavage site (i.e., residue 216 in IgG, taking the first residue of heavy chain constant region to be 114) and ending at the C-terminus of the antibody. Accordingly, a complete Fc region comprises at least a hinge domain, a CH2 domain, and a CH3 domain.

The term “native Fc” or “wild-type Fc,” as used herein, refers to a molecule comprising the sequence of a non-antigen-binding fragment resulting from digestion of an antibody or produced by other means, whether in monomeric or multimeric form, and can contain the hinge region. The original immunoglobulin source of the native Fc is typically of human origin and can be any of the immunoglobulins, such as IgG1 and IgG2. Native Fc molecules are made up of monomeric polypeptides that can be linked into dimeric or multimeric forms by covalent (i.e., disulfide bonds) and non-covalent association. The number of intermolecular disulfide bonds between monomeric subunits of native Fc molecules ranges from 1 to 4 depending on class (e.g., IgG, IgA, and IgE) or subclass (e.g., IgG1, IgG2, IgG3, IgA1, and IgGA2). One example of a native Fc is a disulfide-bonded dimer resulting from papain digestion of an IgG. The term “native Fc,” as used herein, is generic to the monomeric, dimeric, and multimeric forms. The term “Fc variant” or “modified Fc,” as used herein, refers to a molecule or sequence that is modified from a native/wild-type Fc but still comprises a binding site for the FcRn. Thus, the term “Fc variant” can comprise a molecule or sequence that is humanized from a non-human native Fc. Furthermore, a native Fc comprises regions that can be removed because they provide structural features or biological activities that are not required for the antibody-like binding polypeptides described herein. Thus, the term “Fc variant” comprises a molecule or sequence that lacks one or more native Fc sites or residues, or in which one or more Fc sites or residues has be modified, that affect or are involved in: (1) disulfide bond formation, (2) incompatibility with a selected host cell, (3)N-terminal heterogeneity upon expression in a selected host cell, (4) glycosylation, (5) interaction with complement, (6) binding to an Fc receptor other than a salvage receptor, or (7) antibody-dependent cellular cytotoxicity (ADCC).

In certain exemplary embodiments, an Fc variant featured herein has one or more of increased serum half-life, enhanced FcRn binding affinity, enhanced FcRn binding affinity at acidic pH, enhanced FcγRIIIa binding affinity, and/or similar thermal stability, as compared to an IgG antibody comprising a wild-type Fc.

The term “Fc domain” as used herein encompasses native/wild-type Fc and Fc variants and sequences as defined above. As with Fc variants and native Fc molecules, the term “Fc domain” includes molecules in monomeric or multimeric form, whether digested from whole antibody or produced by other means.

As indicated above, the variable regions of an antibody allow it to selectively recognize and specifically bind epitopes on antigens. That is, the VL domain and VH domain of an antibody combine to form the variable region (Fv) that defines a three-dimensional antigen binding site. This quaternary antibody structure forms the antigen binding site present at the end of each arm of the Y. More specifically, the antigen binding site is defined by three complementary determining regions (CDRs) on each of the heavy and light chain variable regions. As used herein, the term “antigen binding site” includes a site that specifically binds (immunoreacts with) an antigen (e.g., a cell surface or soluble antigen). The antigen binding site includes an immunoglobulin heavy chain and light chain variable region and the binding site formed by these variable regions determines the specificity of the antibody. An antigen binding site is formed by variable regions that vary from one antibody to another. The altered antibodies of the current disclosure comprise at least one antigen binding site.

In certain embodiments, binding polypeptides of the current disclosure comprise at least two antigen binding domains that provide for the association of the binding polypeptide with the selected antigen. The antigen binding domains need not be derived from the same immunoglobulin molecule. In this regard, the variable region may or be derived from any type of animal that can be induced to mount a humoral response and generate immunoglobulins against the desired antigen. As such, the variable region of a binding polypeptide may be, for example, of mammalian origin e.g., may be human, murine, rat, goat, sheep, non-human primate (such as cynomolgus monkeys, macaques, etc.), lupine, or camelid (e.g., from camels, llamas and related species).

In naturally occurring antibodies, the six CDRs present on each monomeric antibody are short, non-contiguous sequences of amino acids that are specifically positioned to form the antigen binding site as the antibody assumes its three-dimensional configuration in an aqueous environment. The remainder of the heavy and light variable domains show less inter-molecular variability in amino acid sequence and are termed the framework regions. The framework regions largely adopt a β-sheet conformation and the CDRs form loops which connect, and in some cases form part of, the β-sheet structure. Thus, these framework regions act to form a scaffold that provides for positioning the six CDRs in correct orientation by inter-chain, non-covalent interactions. The antigen binding domain formed by the positioned CDRs defines a surface complementary to the epitope on the immunoreactive antigen. This complementary surface promotes the non-covalent binding of the antibody to the immunoreactive antigen epitope.

Exemplary binding polypeptides include antibody variants. As used herein, the term “antibody variant” includes synthetic and engineered forms of antibodies which are altered such that they are not naturally occurring, e.g., antibodies that comprise at least two heavy chain portions but not two complete heavy chains (such as, domain deleted antibodies or minibodies); multi-specific forms of antibodies (e.g., bi-specific, tri-specific, etc.) altered to bind to two or more different antigens or to different epitopes on a single antigen); heavy chain molecules joined to scFv molecules and the like. In addition, the term “antibody variant” includes multivalent forms of antibodies (e.g., trivalent, tetravalent, etc., antibodies that bind to three, four or more copies of the same antigen.

As used herein the term “valency” refers to the number of potential target binding sites in a polypeptide. Each target binding site specifically binds one target molecule or specific site on a target molecule. When a polypeptide comprises more than one target binding site, each target binding site may specifically bind the same or different molecules (e.g., may bind to different ligands or different antigens, or different epitopes on the same antigen). The subject binding polypeptides typically has at least one binding site specific for a human antigen molecule.

The term “specificity” refers to the ability to specifically bind (e.g., immunoreact with) a given target antigen (e.g., a human target antigen). A binding polypeptide may be monospecific and contain one or more binding sites which specifically bind a target or a polypeptide may be multi-specific and contain two or more binding sites which specifically bind the same or different targets. In certain embodiments, a binding polypeptide is specific for two different (e.g., non-overlapping) portions of the same target. In certain embodiments, a binding polypeptide is specific for more than one target. Exemplary binding polypeptides (e.g., antibodies) which comprise antigen binding sites that bind to antigens expressed on tumor cells are known in the art and one or more CDRs from such antibodies can be included in an antibody as described herein.

The term “antigen” or “target antigen,” as used herein, refers to a molecule or a portion of a molecule that is capable of being bound by the binding site of a binding polypeptide. A target antigen may have one or more epitopes.

The term “about” or “approximately” means within about 20%, such as within about 10%, within about 5%, or within about 1% or less of a given value or range.

As used herein, “administer” or “administration” refers to the act of injecting or otherwise physically delivering a substance as it exists outside the body (e.g., an isolated binding polypeptide provided herein) into a patient, such as by, but not limited to, pulmonary (e.g., inhalation), mucosal (e.g., intranasal), intradermal, intravenous, intramuscular delivery and/or any other method of physical delivery described herein or known in the art. When a disease, or a symptom thereof, is being managed or treated, administration of the substance typically occurs after the onset of the disease or symptoms thereof. When a disease, or symptom thereof, is being prevented, administration of the substance typically occurs before the onset of the disease or symptoms thereof and may be continued chronically to defer or reduce the appearance or magnitude of disease-associated symptoms.

As used herein, the term “composition” is intended to encompass a product containing the specified ingredients (e.g., an isolated binding polypeptide provided herein) in, optionally, the specified amounts, as well as any product which results, directly or indirectly, from combination of the specified ingredients in, optionally, the specified amounts.

“Effective amount” means the amount of active pharmaceutical agent (e.g., an isolated binding polypeptide of the present disclosure) sufficient to effectuate a desired physiological outcome in an individual in need of the agent. The effective amount may vary among individuals depending on the health and physical condition of the individual to be treated, the taxonomic group of the individuals to be treated, the formulation of the composition, assessment of the individual's medical condition, and other relevant factors.

As used herein, the terms “subject” and “patient” are used interchangeably. As used herein, a subject is can be a mammal, such as a non-primate (e.g., cows, pigs, horses, cats, dogs, rats, etc.) or a primate (e.g., monkey and human). In certain embodiments, the term “subject,” as used herein, refers to a vertebrate, such as a mammal. Mammals include, without limitation, humans, non-human primates, wild animals, feral animals, farm animals, sport animals, and pets.

As used herein, the term “therapy” refers to any protocol, method and/or agent that can be used in the prevention, management, treatment and/or amelioration of a disease or a symptom related thereto. In some embodiments, the term “therapy” refers to any protocol, method and/or agent that can be used in the modulation of an immune response to an infection in a subject or a symptom related thereto. In some embodiments, the terms “therapies” and “therapy” refer to a biological therapy, supportive therapy, and/or other therapies useful in the prevention, management, treatment and/or amelioration of a disease or a symptom related thereto, known to one of skill in the art such as medical personnel. In other embodiments, the terms “therapies” and “therapy” refer to a biological therapy, supportive therapy, and/or other therapies useful in the modulation of an immune response to an infection in a subject or a symptom related thereto known to one of skill in the art such as medical personnel.

As used herein, the terms “treat,” “treatment” and “treating” refer to the reduction or amelioration of the progression, severity, and/or duration of a disease or a symptom related thereto, resulting from the administration of one or more therapies (including, but not limited to, the administration of one or more prophylactic or therapeutic agents, such as an isolated binding polypeptide provided herein). The term “treating,” as used herein, can also refer to altering the disease course of the subject being treated. Therapeutic effects of treatment include, without limitation, preventing occurrence or recurrence of disease, alleviation of symptom(s), diminishment of direct or indirect pathological consequences of the disease, decreasing the rate of disease progression, amelioration or palliation of the disease state, and remission or improved prognosis.

Binding Polypeptides

In one aspect, the present disclosure provides binding polypeptides (e.g., antibodies, immunoadhesins, antibody variants, and fusion proteins) comprising a modified Fc domain.

The binding polypeptides disclosed herein encompass any binding polypeptide that comprises a modified Fc domain. In certain embodiments, the binding polypeptide is an antibody, or immunoadhesin or derivative thereof. Any antibody from any source or species can be employed in the binding polypeptides disclosed herein. Suitable antibodies include without limitation, human antibodies, humanized antibodies, or chimeric antibodies. Suitable antibodies include without limitation, monoclonal antibodies, polyclonal antibodies, full-length antibodies, or single chain antibodies.

Fc domains from any immunoglobulin class (e.g., IgM, IgG, IgD, IgA and IgE) and species can be used in the binding polypeptides disclosed herein. Chimeric Fc domains comprising portions of Fc domains from different species or Ig classes can also be employed. In certain embodiments, the Fc domain is a human Fc domain. In some embodiments, the Fc domain is an IgG1 Fc domain. In other embodiments, the Fc domain is an IgG4 Fc domain. In some embodiments, the Fc domain is a human IgG1 or IgG4 Fc domain. In some embodiments, the Fc domain is a human IgG1 Fc domain. In the case of Fc domains of other species and/or Ig classes or isotypes, the skilled artisan will appreciate that any of the amino acid substitutions described herein can be adapted accordingly. In some embodiments, the modified Fc domain may comprise an amino acid substitution selected from M252, 1253, S254, T256, K288, T307, K322, E380, L432, N434, or Y436, and any combinations thereof, according to EU numbering. In some embodiments, the modified Fc domain may comprise a double amino acid substitution at any two amino acid positions selected from M252, 1253, S254, T256, K288, T307, K322, E380, L432, N434, and Y436, according to EU numbering. In some embodiments, the modified Fc domain may comprise a triple amino acid substitution at any three amino acid positions selected from M252, 1253, S254, T256, K288, T307, K322, E380, L432, N434, and Y436, according to EU numbering. In some embodiments, the modified Fc domain may comprise a quadruple amino acid substitution at any four amino acid positions selected from M252, 1253, S254, T256, K288, T307, K322, E380, L432, N434, and Y436, according to EU numbering. In some embodiments, it may be desirable for a modified Fc domain to comprise an amino acid substitution at any of the amino acid positions selected from M252, 1253, S254, T256, K288, T307, K322, E380, L432, or Y436, and any combinations thereof, wherein amino acid position N434 is not substituted (i.e., amino acid position N434 is wild-type), according to EU numbering.

In some embodiments, the modified Fc domain may comprise an amino acid substitution selected from M252Y (i.e., a tyrosine at amino acid position 252), T256D, T256E, K288D, K288N, T307A, T307E, T307F, T307M, T307Q, T307W, E380C, N434F, N434P, N434Y, Y436H, Y436N, or Y436W, and any combinations thereof, according to EU numbering. In some embodiments, the modified Fc domain may comprise a double amino acid substitution selected from M252, wherein the substitution is M252Y; T256, wherein the substitution is T256D, or T256E; K288, wherein the substitution is K288D, or K288N; T307, wherein the substitution is T307A, T307E, T307F, T307M, T307Q, or T307W; E380, wherein the substitution is E380C; N434, wherein the substitution is N434F, N434P, or N434Y; Y436, wherein the substitution is Y436H, Y436N, or Y436W, according to EU numbering. In some embodiments, the modified Fc domain may comprise a triple amino acid substitution selected from M252, wherein the substitution is M252Y; T256, wherein the substitution is T256D, or T256E; K288, wherein the substitution is K288D, or K288N; T307, wherein the substitution is T307A, T307E, T307F, T307M, T307Q, or T307W; E380, wherein the substitution is E380C; N434, wherein the substitution is N434F, N434P, or N434Y; Y436, wherein the substitution is Y436H, Y436N, or Y436W, according to EU numbering. In some embodiments, the modified Fc domain may comprise a quadruple amino acid substitution selected from M252, wherein the substitution is M252Y; T256, wherein the substitution is T256D, or T256E; K288, wherein the substitution is K288D, or K288N; T307, wherein the substitution is T307A, T307E, T307F, T307M, T307Q, or T307W; E380, wherein the substitution is E380C; N434, wherein the substitution is N434F, N434P, or N434Y; Y436, wherein the substitution is Y436H, Y436N, or Y436W, according to EU numbering. In some embodiments, it may be desirable for a modified Fc domain to comprise an amino acid substitution at any of the amino acid positions selected from M252Y, T256D, T256E, K288D, K288N, T307A, T307E, T307F, T307M, T307Q, T307W, E380C, Y436H, Y436N, or Y436W, and any combinations thereof, wherein amino acid position N434 is not substituted with a phenylalanine (F) or a tyrosine (Y), according to EU numbering. In some embodiments, it may be desirable for a modified Fc domain to comprise an amino acid substitution at any of the amino acid positions selected from M252Y, T256D, T256E, K288D, K288N, T307A, T307E, T307F, T307M, T307Q, T307W, E380C, Y436H, Y436N, or Y436W, and any combinations thereof, wherein amino acid position N434 is not substituted with a tyrosine (Y), according to EU numbering. In some embodiments, it may be desirable for a modified Fc domain to comprise an amino acid substitution at any of the amino acid positions selected from M252Y, T256D, T256E, K288D, K288N, T307A, T307E, T307F, T307M, T307Q, T307W, E380C, Y436H, Y436N, or Y436W, and any combinations thereof, wherein amino acid position N434 is not substituted (i.e., amino acid position N434 is wild-type), according to EU numbering.

In certain embodiments, the modified Fc domain may comprise an amino acid substitution selected from M252, T256, T307, or N434, and any combinations thereof, according to EU numbering. In certain embodiments, the modified Fc domain may comprise a double amino acid substitution at any two amino acid positions selected from M252, T256, T307, and N434, according to EU numbering. In certain embodiments, the modified Fc domain may comprise a triple amino acid substitution at any three amino acid positions selected from M252, T256, T307, and N434, according to EU numbering. In certain embodiments, the modified Fc domain may comprise a quadruple amino acid substitution at amino acid positions M252, T256, T307, and N434, according to EU numbering. In some embodiments, it may be desirable for a modified Fc domain to comprise an amino acid substitution selected from M252, T256, or T307, and any combinations thereof, wherein amino acid position N434 is not substituted (i.e., amino acid position N434 is wild-type), according to EU numbering.

In exemplary embodiments, the modified Fc domain may comprise an amino acid substitution selected from M252, wherein the substitution is M252Y; T256, wherein the substitution is T256D, or T256E; T307, wherein the substitution is T307Q, or T307W; or N434, wherein the substitution is N434F, or N434Y, and any combinations thereof, according to EU numbering. In certain embodiments, the modified Fc domain may comprise a double amino acid substitution at any two amino acid positions selected from M252, wherein the substitution is M252Y; T256, wherein the substitution is T256D, or T256E; T307, wherein the substitution is T307Q, or T307W; or N434, wherein the substitution is N434F, or N434Y, according to EU numbering. In certain embodiments, the modified Fc domain may comprise a triple amino acid substitution at any three amino acid positions selected from M252, wherein the substitution is M252Y; T256, wherein the substitution is T256D, or T256E; T307, wherein the substitution is T307Q, or T307W; or N434, wherein the substitution is N434F, or N434Y, according to EU numbering. In certain embodiments, the modified Fc domain may comprise a quadruple amino acid substitution at amino acid positions selected from M252, wherein the substitution is M252Y; T256, wherein the substitution is T256D, or T256E; T307, wherein the substitution is T307Q, or T307W; or N434, wherein the substitution is N434F, or N434Y, according to EU numbering. In some embodiments, it may be desirable for a modified Fc domain to comprise an amino acid substitution selected from M252Y, T256D, T256E, T307Q, or T307W, and any combinations thereof, wherein amino acid position N434 is not substituted with a phenylalanine (F) or a tyrosine (Y), according to EU numbering. In some embodiments, it may be desirable for a modified Fc domain to comprise an amino acid substitution selected from M252Y, T256D, T256E, T307Q, or T307W, and any combinations thereof, wherein amino acid position N434 is not substituted with a tyrosine (Y), according to EU numbering. In some embodiments, it may be desirable for a modified Fc domain to comprise an amino acid substitution selected from M252Y, T256D, T256E, T307Q, or T307W, and any combinations thereof, wherein amino acid position N434 is not substituted (i.e., amino acid position N434 is wild-type), according to EU numbering.

In certain embodiments, the modified Fc domain may comprise an amino acid substitution selected from T256D, or T256E, and/or T307W, or T307Q, and further comprises an amino acid substitution selected from N434F, or N434Y, or M252Y, according to EU numbering. In some embodiments, it may be desirable for a modified Fc domain to comprise an amino acid substitution selected from T256D, or T256E, and/or T307W, or T307Q, and further comprise the amino acid substitution M252Y, wherein amino acid position N434 is not substituted with a phenylalanine (F) or a tyrosine (Y), according to EU numbering. In some embodiments, it may be desirable for a modified Fc domain to comprise an amino acid substitution selected from T256D, or T256E, and/or T307W, or T307Q, and further comprise the amino acid substitution M252Y, wherein amino acid position N434 is not substituted with a tyrosine (Y), according to EU numbering. In some embodiments, it may be desirable for a modified Fc domain to comprise an amino acid substitution selected from T256D, or T256E, and/or T307W, or T307Q, and further comprise the amino acid substitution M252Y, wherein amino acid position N434 is not substituted (i.e., amino acid position N434 is wild-type), according to EU numbering.

In some embodiments, the modified Fc domain may comprise a double amino acid substitution selected from M252Y/T256D, M252Y/T256E, M252Y/T307Q, M252Y/T307W, M252Y/N434F, M252Y/N434Y, T256D/T307Q, T256D/T307W, T256D/N434F, T256D/N434Y, T256E/T307Q, T256E/T307W, T256E/N434F, T256E/N434Y, T307Q/N434F, T307Q/N434Y, T307W/N434F, and T307W/N434Y, according to EU numbering. In some embodiments, the modified Fc domain may comprise a triple amino acid substitution selected from M252Y/T256D/T307Q, M252Y/T256D/T307W, M252Y/T256D/N434F, M252Y/T256D/N434Y, M252Y/T256E/T307Q, M252Y/T256E/T307W, M252Y/T256E/N434F, M252Y/T256E/N434Y, M252Y/T307Q/N434F, M252Y/T307Q/N434Y, M252Y/T307W/N434F, M252T/T307W/N434Y, T256D/307Q/N434F, T256D/307W/N434F, T256D/307Q/N434Y, T256D/307W/N434Y, T256E/307Q/N434F, T256E/307W/N434F, T256E/307Q/N434Y, and T256E/307W/N434Y, according to EU numbering.

In some embodiments, the modified Fc domain may comprise a quadruple amino acid substitution selected from M252Y/T256D/T307Q/N434F, M252Y/T256E/T307Q/N434F, M252Y/T256D/T307W/N434F, M252Y/T256E/T307W/N434F, M252Y/T256D/T307Q/N434Y, M252Y/T256E/T307Q/N434Y, M252Y/T256D/T307W/N434Y, and M252Y/T256E/T307W/N434Y, according to EU numbering.

In some embodiments, it may be desirable for a modified Fc domain to comprise a wild-type amino acid at amino acid position N434, according to EU numbering. In some embodiments, it may be desirable for an Fc domain to not comprise a phenylalanine (F) or tyrosine (Y) at amino acid position N434, according to EU numbering. In some embodiments, it may be desirable for an Fc domain to not comprise a tyrosine (Y) at amino acid position N434, according to EU numbering. In some embodiments, the modified Fc domain may comprise a double amino acid substitution selected from M252Y/T256D, M252Y/T256E, M252Y/T307Q, M252Y/T307W, T256D/T307Q, T256D/T307W, T256E/T307Q, and T256E/T307W, according to EU numbering. In some embodiments, the modified Fc domain may comprise a triple amino acid substitution selected from M252Y/T256D/T307Q, M252Y/T256D/T307W, M252Y/T256E/T307Q, and M252Y/T256E/T307W, according to EU numbering.

In one embodiment, a binding polypeptide with altered FcRn binding comprises an Fc domain having one or more amino acid substitutions as disclosed herein. In one embodiment, a binding polypeptide with enhanced FcRn binding affinity comprises an Fc domain having one or more amino acid substitutions as disclosed herein. In one embodiment, a binding polypeptide with enhanced FcRn binding affinity comprises an Fc domain having two or more amino acid substitutions as disclosed herein. In one embodiment, a binding polypeptide with enhanced FcRn binding affinity comprises an Fc domain having three or more amino acid substitutions as disclosed herein.

In some embodiments, a binding polypeptide may exhibit a species-specific FcRn binding affinity. In one embodiment, a binding polypeptide may exhibit human FcRn binding affinity. In one embodiment, a binding polypeptide may exhibit rat FcRn binding affinity. In some embodiments, a binding polypeptide may exhibit cross-species FcRn binding affinity. Such binding polypeptides are said to be cross-reactive across one or more different species. In one embodiment, a binding polypeptide may exhibit both human and rat FcRn binding affinity.

The neonatal Fc receptor (FcRn) interacts with the Fc region of antibodies to promote recycling through rescue of normal lysosomal degradation. This process is a pH-dependent process that occurs in the endosomes at acidic pH (e.g., a pH less than 6.5) but not under the physiological pH conditions of the bloodstream (e.g., a non-acidic pH). In some embodiments, a binding polypeptide of the present disclosure comprising a modified Fc domain has enhanced FcRn binding affinity at an acidic pH compared to a binding polypeptide comprising a wild-type Fc domain. In some embodiments, a binding polypeptide comprising a modified Fc domain has enhanced FcRn binding affinity at pH less than 7, e.g., at about pH 6.5, at about pH 6.0, at about pH 5.5, at about pH 5.0, compared to a binding polypeptide comprising a wild-type Fc domain. In some embodiments, a binding polypeptide comprising a modified Fc domain has enhanced FcRn binding affinity at pH less than 7, e.g., at about pH 6.5, at about pH 6.0, at about pH 5.5, at about pH 5.0, compared to the FcRn binding affinity of the binding polypeptide at an elevated non-acidic pH. An elevated non-acidic pH can be, e.g., pH greater than 7, about pH 7, about pH 7.4, about pH 7.6, about pH 7.8, about pH 8.0, about pH 8.5, about pH 9.0. In certain embodiments, it may be desired for a binding polypeptide comprising a modified Fc domain to exhibit approximately the same FcRn binding affinity at non-acidic pH as a binding polypeptide comprising a wild-type Fc domain. In some embodiments, it may be desired for a binding polypeptide comprising a modified Fc domain to exhibit less FcRn binding affinity at non-acidic pH than a binding polypeptide comprising a modified Fc domain having the double amino acid substitution M428L/N434S, according to EU numbering. Accordingly, it may be desired for a binding polypeptide comprising a modified Fc domain to exhibit minimal perturbation to pH-dependent FcRn binding.

In some embodiments, a binding polypeptide comprising a modified Fc domain having enhanced FcRn binding affinity at an acidic pH, has a reduced (i.e., slower) FcRn off-rate as compared to a binding polypeptide comprising a wild-type Fc domain. In some embodiments, a binding polypeptide comprising a modified Fc domain having enhanced FcRn binding affinity at an acidic pH compared to the FcRn binding affinity of the binding polypeptide at an elevated non-acidic pH, has a slower FcRn off-rate at the acidic pH compared to the FcRn off-rate of the binding polypeptide at the elevated non-acidic pH.

In some embodiments, a binding polypeptide comprising a modified Fc domain that exhibits higher FcRn binding affinity at non-acidic pH compared to a binding polypeptide comprising a wild-type Fc domain is provided. In some embodiments, a binding polypeptide comprising a modified Fc domain that exhibits higher FcRn binding affinity at acidic pH compared to a binding polypeptide comprising a wild-type Fc domain is provided. In some embodiments, a binding polypeptide comprising a modified Fc domain that exhibits higher FcRn binding affinity at non-acidic pH compared to a binding polypeptide comprising a wild-type Fc domain, and exhibits higher FcRn binding affinity at acidic pH compared to a binding polypeptide comprising a wild-type Fc domain is provided. Accordingly, in certain embodiments, a binding polypeptide comprising a modified Fc domain that exhibits loss of pH-dependent FcRn binding is provided.

Certain embodiments include antibodies which, in addition to the Fc mutations described herein that exhibit altered FcRn binding affinity, comprise at least one amino acid in one or more of the constant region domains and/or at least one amino acid in one or more of the variable region domains that has been deleted or otherwise altered so as to provide desired biochemical characteristics such as, e.g., reduced or enhanced effector functions, the ability to non-covalently dimerize, increased ability to localize at the site of a tumor, reduced serum half-life, increased serum half-life when compared with a whole, unaltered antibody of approximately the same immunogenicity and the like.

In certain other embodiments, binding polypeptides comprise constant regions derived from different antibody isotypes (e.g., constant regions from two or more of a human IgG1, IgG2, IgG3, or IgG4). In other embodiments, binding polypeptides comprise a chimeric hinge (i.e., a hinge comprising hinge portions derived from hinge domains of different antibody isotypes, e.g., an upper hinge domain from an IgG4 molecule and an IgG1 middle hinge domain).

In certain embodiments, the Fc domain may be mutated to increase or decrease effector function using techniques known in the art. In some embodiments, a binding polypeptide of the present disclosure comprising a modified Fc domain has altered binding affinity to an Fc receptor. There are several different types of Fc receptors, which are classified based on the type of antibody that they recognize. For example, Fc-gamma receptors (FcγR) bind to IgG class antibodies, Fc-alpha receptors (FcαR) bind to IgA class antibodies, and Fc-epsilon receptors (FccR) bind to IgE class antibodies. The FcγRs belong to a family that includes several members, e.g., FcγRI, FcγRIIa, FcγRIIb, FcγRIIIa, and FcγRIIIb. In some embodiments, a binding polypeptide comprising a modified Fc domain has altered FcγRIIIa binding affinity, compared to a binding polypeptide comprising a wild-type Fc domain. In some embodiments, a binding polypeptide comprising a modified Fc domain has reduced FcγRIIIa binding affinity, compared to a binding polypeptide comprising a wild-type Fc domain. In some embodiments, a binding polypeptide comprising a modified Fc domain has enhanced FcγRIIIa binding affinity, compared to a binding polypeptide comprising a wild-type Fc domain. In some embodiments, a binding polypeptide comprising a modified Fc domain has approximately the same FcγRIIIa binding affinity, compared to a binding polypeptide comprising a wild-type Fc domain.

In other embodiments, binding polypeptides, for use in the diagnostic and treatment methods described herein have a constant region, e.g., an IgG1 heavy chain constant region, which is altered to reduce or eliminate glycosylation. For example, binding polypeptides (e.g., antibodies or immunoadhesins) comprising a modified Fc domain may further comprise an amino acid substitution which alters the glycosylation of the antibody Fc. For example, said modified Fc domain may have reduced glycosylation (e.g., N- or O-linked glycosylation).

Exemplary amino acid substitutions which confer reduced or altered glycosylation are disclosed in International PCT Publication No. WO05/018572, which is incorporated in its entirety by reference herein. In some embodiments, the binding polypeptides are modified to eliminate glycosylation. Such binding polypeptides may be referred to as “agly” binding polypeptides (e.g., “agly” antibodies). While not being bound by theory, it is believed that “agly” binding polypeptides may have an improved safety and stability profile in vivo. Agly binding polypeptides can be of any isotype or subclass thereof, e.g., IgG1, IgG2, IgG3, or IgG4. Numerous art-recognized methods are available for making “agly” antibodies or antibodies with altered glycans. For example, genetically engineered host cells (e.g., modified yeast, e.g., Picchia, or CHO cells) with modified glycosylation pathways (e.g., glycosyl-transferase deletions) can be used to produce such antibodies.

In certain embodiments, binding polypeptides may comprise an antibody constant region (e.g., an IgG constant region e.g., a human IgG constant region, e.g., a human IgG1 constant region) which mediates one or more effector functions. For example, binding of the C1-complex to an antibody constant region may activate the complement system. Activation of the complement system is important in the opsonization and lysis of cell pathogens. The activation of the complement system also stimulates the inflammatory response and may also be involved in autoimmune hypersensitivity. Further, antibodies bind to receptors on various cells via the Fc domain (Fc receptor binding sites on the antibody Fc region bind to Fc receptors (FcRs) on a cell). There are a number of Fc receptors which are specific for different classes of antibody, including IgG (gamma receptors), IgE (epsilon receptors), IgA (alpha receptors) and IgM (mu receptors). Binding of antibody to Fc receptors on cell surfaces triggers a number of important and diverse biological responses including engulfment and destruction of antibody-coated particles, clearance of immune complexes, lysis of antibody-coated target cells by killer cells (called antibody-dependent cell-mediated cytotoxicity, or ADCC), release of inflammatory mediators, placental transfer and control of immunoglobulin production. In some embodiments, the binding polypeptides (e.g., antibodies or immunoadhesins) bind to an Fc-gamma receptor. In alternative embodiments, binding polypeptides may comprise a constant region which is devoid of one or more effector functions (e.g., ADCC activity) and/or is unable to bind Fcγ receptor.

Proteins, including antibodies, with low thermodynamic stability have an increased propensity for misfolding and aggregation and would limit or hinder the activity, efficacy, and potential of the protein as a useful therapeutic. In certain embodiments, a binding polypeptide comprising a modified Fc domain has approximately the same thermal stability as a binding polypeptide comprising a wild-type Fc domain. In some embodiments, a binding polypeptide comprising a modified Fc domain has approximately the same thermal stability as a binding polypeptide comprising a modified Fc domain having the triple amino acid substation M252Y/S254T/T256E (YTE).

The resulting physiological profile, bioavailability and other biochemical effects of the modifications, such as tumor localization, biodistribution and serum half-life, may easily be measured and quantified using well-known immunological techniques without undue experimentation.

In certain embodiments, the binding polypeptide of the current disclosure may comprise an antigen binding fragment of an antibody. The term “antigen-binding fragment” refers to a polypeptide fragment of an immunoglobulin or antibody which binds antigen or competes with intact antibody (i.e., with the intact antibody from which they were derived) for antigen binding (i.e., specific binding). Antigen binding fragments can be produced by recombinant or biochemical methods that are well known in the art. Exemplary antigen-binding fragments include Fv, Fab, Fab′, and (Fab′)2. In exemplary embodiments, a binding polypeptide of the current disclosure comprises an antigen binding fragment and a modified Fc domain.

In some embodiments, the binding polypeptide comprises a single chain variable region sequence (ScFv). Single chain variable region sequences comprise a single polypeptide having one or more antigen binding sites, e.g., a VL domain linked by a flexible linker to a VH domain. ScFv molecules can be constructed in a VH-linker-VL orientation or VL-linker-VH orientation. The flexible hinge that links the VL and VH domains that make up the antigen binding site includes from about 10 to about 50 amino acid residues. Connecting peptides are known in the art. Binding polypeptides may comprise at least one scFv and/or at least one constant region. In one embodiment, a binding polypeptide of the current disclosure may comprise at least one scFv linked or fused to a modified Fc domain.

In some embodiments, a binding polypeptide of the current disclosure is a multivalent (e.g., tetravalent) antibody which is produced by fusing a DNA sequence encoding an antibody with a ScFv molecule (e.g., an altered ScFv molecule). For example, in one embodiment, these sequences are combined such that the ScFv molecule (e.g., an altered ScFv molecule) is linked at its N-terminus or C-terminus to an Fc fragment of an antibody via a flexible linker (e.g., a gly/ser linker). In another embodiment a tetravalent antibody of the current disclosure can be made by fusing an ScFv molecule to a connecting peptide, which is fused to a modified Fc domain to construct an ScFv-Fab tetravalent molecule.

In another embodiment, a binding polypeptide of the current disclosure is an altered minibody. An altered minibody of the current disclosure is a dimeric molecule made up of two polypeptide chains each comprising an ScFv molecule which is fused to a modified Fc domain via a connecting peptide. Minibodies can be made by constructing an ScFv component and connecting peptide components using methods described in the art (see, e.g., U.S. Pat. No. 5,837,821 or WO 94/09817A1). In another embodiment, a tetravalent minibody can be constructed. Tetravalent minibodies can be constructed in the same manner as minibodies, except that two ScFv molecules are linked using a flexible linker. The linked scFv-scFv construct is then joined to a modified Fc domain.

In another embodiment, a binding polypeptide of the current disclosure comprises a diabody. Diabodies are dimeric, tetravalent molecules each having a polypeptide similar to scFv molecules, but usually having a short (less than 10, e.g., about 1 to about 5) amino acid residue linker connecting both variable domains, such that the VL and VH domains on the same polypeptide chain cannot interact. Instead, the VL and VH domain of one polypeptide chain interact with the VH and VL domain (respectively) on a second polypeptide chain (see, for example, WO 02/02781). Diabodies of the current disclosure comprise an scFv-like molecule fused to a modified Fc domain.

In other embodiments, the binding polypeptides comprise multi-specific or multivalent antibodies comprising one or more variable domain in series on the same polypeptide chain, e.g., tandem variable domain (TVD) polypeptides. Exemplary TVD polypeptides include the “double head” or “Dual-Fv” configuration described in U.S. Pat. No. 5,989,830. In the Dual-Fv configuration, the variable domains of two different antibodies are expressed in a tandem orientation on two separate chains (one heavy chain and one light chain), wherein one polypeptide chain has two VH domains in series separated by a peptide linker (VH1-linker-VH2) and the other polypeptide chain consists of complementary VL domains connected in series by a peptide linker (VL1-linker-VL2). In the cross-over double head configuration, the variable domains of two different antibodies are expressed in a tandem orientation on two separate polypeptide chains (one heavy chain and one light chain), wherein one polypeptide chain has two VH domains in series separated by a peptide linker (VH1-linker-VH2) and the other polypeptide chain consists of complementary VL domains connected in series by a peptide linker in the opposite orientation (VL2-linker-VL1). Additional antibody variants based on the “Dual-Fv” format include the Dual-Variable-Domain IgG (DVD-IgG) bispecific antibody (see U.S. Pat. No. 7,612,181 and the TBTI format (see US 2010/0226923 A1). In some embodiments, binding polypeptides comprise multi-specific or multivalent antibodies comprising one or more variable domain in series on the same polypeptide chain fused to a modified Fc domain.

In another exemplary embodiment, the binding polypeptide comprises a cross-over dual variable domain IgG (CODV-IgG) bispecific antibody based on a “double head” configuration (see US20120251541 A1, which is incorporated by reference herein in its entirety).

In another exemplary embodiment, the binding polypeptide is an immunoadhesin. As used herein, an “immunoadhesin” refers to a binding polypeptide comprising one or more binding domains (e.g., from a receptor, ligand, or cell-adhesion molecule) linked to an immunoglobulin constant domain (i.e., an Fc region) (see e.g., Ashkenazi et al. 1995, Methods 8(2): 104-115, and Isaacs (1997) Brit. J. Rheum. 36:305 which are incorporated by reference herein in their entireties). Immunoadhesins are identified by the suffix “-cept” in their international nonproprietary names (INN). Like antibodies, immunoadhesins have long circulating half-lives, are readily purified by affinity-based methods, and have avidity advantages conferred by bivalency. Examples commercially available therapeutic immunoadhesins include etanercept (ENBREL®), abatacept (ORENCIA®), rilonacept (ARCALYST®), aflibercept (ZALTRAP®/EYLEA®), and belatacept (NULOJIX®).

In certain embodiments, the binding polypeptide comprises immunoglobulin-like domains. Suitable immunoglobulin-like domains include, without limitation, fibronectin domains (see, for example, Koide et al. (2007), Methods Mol. Biol. 352: 95-109, which is incorporated by reference herein in its entirety), DARPin (see, for example, Stumpp et al. (2008) Drug Discov. Today 13 (15-16): 695-701, which is incorporated by reference herein in its entirety), Z domains of protein A (see, Nygren et al. (2008) FEBS J. 275 (11): 2668-76, which is incorporated by reference herein in its entirety), Lipocalins (see, for example, Skerra et al. (2008) FEBS J. 275 (11): 2677-83, which is incorporated by reference herein in its entirety), Affilins (see, for example, Ebersbach et al. (2007) J. Mol. Biol. 372 (1): 172-85, which is incorporated by reference herein in its entirety), Affitins (see, for example, Krehenbrink et al. (2008). J. Mol. Biol. 383 (5): 1058-68, which is incorporated by reference herein in its entirety), Avimers (see, for example, Silverman et al. (2005) Nat. Biotechnol. 23 (12): 1556-61, which is incorporated by reference herein in its entirety), Fynomers, (see, for example, Grabulovski et al. (2007) J Biol Chem 282 (5): 3196-3204, which is incorporated by reference herein in its entirety), and Kunitz domain peptides (see, for example, Nixon et al. (2006) Curr Opin Drug Discov Devel 9 (2): 261-8, which is incorporated by reference herein in its entirety).

For binding polypeptides and immunoadhesins of the present disclosure, virtually any antigen may be targeted by the binding polypeptides, including but not limited to proteins, subunits, domains, motifs, and/or epitopes of target antigens, which includes both soluble factors such as cytokines and membrane-bound factors, and transmembrane receptors.

A binding polypeptide of the present disclosure, comprising a modified Fc domain described herein, can include the CDR sequences or the variable domain sequences of a known “parent” antibody. In some embodiments, the parent antibody and the antibody of the disclosure can share similar or identical sequences except for modifications to the Fc domain as disclosed herein.

### Nucleic Acids and Expression Vectors

In one aspect, the invention provides polynucleotides encoding the binding polypeptides disclosed herein. Methods of making a binding polypeptide comprising expressing these polynucleotides are also provided.

Polynucleotides encoding the binding polypeptides disclosed herein are typically inserted in an expression vector for introduction into host cells that may be used to produce the desired quantity of the claimed antibodies, or immunoadhesins. Accordingly, in certain aspects, the invention provides expression vectors comprising polynucleotides disclosed herein and host cells comprising these vectors and polynucleotides.

The term “vector” or “expression vector” is used herein for the purposes of the specification and claims, to mean vectors used for introducing into and expressing a desired gene in a cell. As known to those skilled in the art, such vectors may easily be selected from the group consisting of plasmids, phages, viruses and retroviruses. In general, a vector will comprise a selection marker, appropriate restriction sites to facilitate cloning of the desired gene and the ability to enter and/or replicate in eukaryotic or prokaryotic cells.

Numerous expression vector systems may be employed. For example, one class of vector utilizes DNA elements which are derived from animal viruses such as bovine papilloma virus, polyoma virus, adenovirus, vaccinia virus, baculovirus, retroviruses (RSV, MMTV or MOMLV), or SV40 virus. Others involve the use of polycistronic systems with internal ribosome binding sites. Additionally, cells which have integrated the DNA into their chromosomes may be selected by introducing one or more markers which allow selection of transfected host cells. The marker may provide for prototrophy to an auxotrophic host, biocide resistance (e.g., antibiotics) or resistance to heavy metals such as copper. The selectable marker gene can either be directly linked to the DNA sequences to be expressed, or introduced into the same cell by co-transformation. Additional elements may also be needed for optimal synthesis of mRNA. These elements may include signal sequences, splice signals, as well as transcriptional promoters, enhancers, and termination signals. In some embodiments the cloned variable region genes are inserted into an expression vector along with the heavy and light chain constant region genes (such as human genes) synthesized as discussed above.

In other embodiments, a binding polypeptide as described herein may be expressed using polycistronic constructs. In such expression systems, multiple gene products of interest such as heavy and light chains of antibodies may be produced from a single polycistronic construct. These systems advantageously use an internal ribosome entry site (IRES) to provide relatively high levels of polypeptides in eukaryotic host cells. Compatible IRES sequences are disclosed in U.S. Pat. No. 6,193,980 which is incorporated by reference herein. Those skilled in the art will appreciate that such expression systems may be used to effectively produce the full range of polypeptides disclosed in the instant application.

More generally, once a vector or DNA sequence encoding a binding polypeptide of the present disclosure, has been prepared, the expression vector may be introduced into an appropriate host cell. That is, the host cell may be transformed. Introduction of the plasmid into the host cell can be accomplished by various techniques well known to those of skill in the art.

These include, but are not limited to, transfection (including electrophoresis and electroporation), protoplast fusion, calcium phosphate precipitation, cell fusion with enveloped DNA, microinjection, and infection with intact virus. See, e.g., Ridgway, A. A. G. “Mammalian Expression Vectors” Chapter 24.2, pp. 470-472 Vectors, Rodriguez and Denhardt, Eds. (Butterworths, Boston, Mass. 1988). The transformed cells are grown under conditions appropriate to the production of the light chains and heavy chains, and assayed for heavy and/or light chain protein synthesis. Exemplary assay techniques include enzyme-linked immunosorbent assay (ELISA), radioimmunoassay (RIA), or fluorescence-activated cell sorter analysis (FACS), immunohistochemistry and the like.

As used herein, the term “transformation” shall be used in a broad sense to refer to the introduction of DNA into a recipient host cell that changes the genotype and consequently results in a change in the recipient cell.

Along those same lines, “host cells” refer to cells that have been transformed with vectors constructed using recombinant DNA techniques and encoding at least one heterologous gene. In descriptions of processes for isolation of polypeptides from recombinant hosts, the terms “cell” and “cell culture” are used interchangeably to denote the source of antibody unless it is clearly specified otherwise. In other words, recovery of polypeptide from the “cells” may mean either from spun down whole cells, or from the cell culture containing both the medium and the suspended cells.

In one embodiment, the host cell line used for expression of the binding polypeptide is of eukaryotic or prokaryotic origin. In one embodiment, the host cell line used for expression of the binding polypeptide is of bacterial origin. In one embodiment, the host cell line used for expression of the binding polypeptide is of mammalian origin; those skilled in the art can determine particular host cell lines which are best suited for the desired gene product to be expressed therein. Exemplary host cell lines include, but are not limited to, DG44 and DUXB11 (Chinese Hamster Ovary lines, DHFR minus), HELA (human cervical carcinoma), CVI (monkey kidney line), COS (a derivative of CVI with SV40 T antigen), R1610 (Chinese hamster fibroblast) BALBC/3T3 (mouse fibroblast), HAK (hamster kidney line), SP2/O (mouse myeloma), BFA-1c1BPT (bovine endothelial cells), RAJI (human lymphocyte), 293 (human kidney). In one embodiment, the cell line provides for altered glycosylation, e.g., afucosylation, of the antibody expressed therefrom (e.g., PER.C6.RTM. (Crucell) or FUT8-knock-out CHO cell lines (POTELLIGENT™ cells) (Biowa, Princeton, N.J.)). In one embodiment NS0 cells may be used. Host cell lines are typically available from commercial services, the American Tissue Culture Collection or from published literature.

In vitro production allows scale-up to give large amounts of the desired binding polypeptides. Techniques for mammalian cell cultivation under tissue culture conditions are known in the art and include homogeneous suspension culture, e.g., in an airlift reactor or in a continuous stirrer reactor, or immobilized or entrapped cell culture, e.g., in hollow fibers, microcapsules, on agarose microbeads or ceramic cartridges. If necessary and/or desired, the solutions of polypeptides can be purified by the customary chromatography methods, for example gel filtration, ion-exchange chromatography, chromatography over DEAE-cellulose and/or (immuno-) affinity chromatography.

One or more genes encoding binding polypeptides can also be expressed in non-mammalian cells such as bacteria or yeast or plant cells. In this regard it will be appreciated that various unicellular non-mammalian microorganisms such as bacteria can also be transformed; i.e. those capable of being grown in cultures or fermentation. Bacteria, which are susceptible to transformation, include members of the enterobacteriaceae, such as strains of Escherichia coli or Salmonella; Bacillaceae, such as Bacillus subtilis; Pneumococcus; Streptococcus, and Haemophilus influenzae. It will further be appreciated that, when expressed in bacteria, the polypeptides can become part of inclusion bodies. The polypeptides must be isolated, purified and then assembled into functional molecules.

In addition to prokaryotes, eukaryotic microbes may also be used. Saccharomyces cerevisiae, or common baker's yeast, is the most commonly used among eukaryotic microorganisms although a number of other strains are commonly available. For expression in Saccharomyces, the plasmid YRp7, for example, (Stinchcomb et al., Nature, 282:39 (1979); Kingsman et al., Gene, 7:141 (1979); Tschemper et al., Gene, 10:157 (1980)) is commonly used. This plasmid already contains the TRP1 gene which provides a selection marker for a mutant strain of yeast lacking the ability to grow in tryptophan, for example ATCC No. 44076 or PEP4-1 (Jones, Genetics, 85:12 (1977)). The presence of the trpI lesion as a characteristic of the yeast host cell genome then provides an effective environment for detecting transformation by growth in the absence of tryptophan.

### Methods of Treatment

In one aspect, the invention provides methods of treating or diagnosing a patient in need thereof comprising administering an effective amount of a binding polypeptide disclosed herein. In certain embodiments, the present disclosure provides kits and methods for the diagnosis and/or treatment of disorders, e.g., neoplastic disorders in a mammalian subject in need of such treatment. In certain exemplary embodiments, the subject is a human.

The binding polypeptides of the current disclosure are useful in a number of different applications. For example, in one embodiment, the subject binding polypeptides are useful for reducing or eliminating cells bearing an epitope recognized by the binding domain of the binding polypeptide. In another embodiment, the subject binding polypeptides are effective in reducing the concentration of or eliminating soluble antigen in the circulation. In another embodiment, the subject binding polypeptides are effective as T-cell engagers. In one embodiment, the binding polypeptides may reduce tumor size, inhibit tumor growth and/or prolong the survival time of tumor-bearing animals. Accordingly, this disclosure also relates to a method of treating tumors in a human or other animal by administering to such human or animal an effective, non-toxic amount of modified antibody.

In one embodiment, the subject binding polypeptides are useful for the treatment of a disease or disorder. For example, the subject binding polypeptides are useful for the treatment of an antibody related disorder, or an antibody responsive disorder, condition, or disease. As used herein, the terms “antibody related disorder” or “antibody responsive disorder” or “condition” or “disease” refer to or describe a disease or disorder that may be ameliorated by the administration of a pharmaceutical composition comprising an antibody or binding polypeptide of the present disclosure.

In one embodiment, the subject binding polypeptides are useful for the treatment of cancer. As used herein, the terms “cancer” or “cancerous” refer to or describe the physiological condition that is typically characterized by unregulated cell growth. Examples of cancer include but are not limited to carcinoma, lymphoma, blastoma, sarcoma (including liposarcoma), neuroendocrine tumors, mesothelioma, schwanoma, meningioma, adenocarcinoma, melanoma, and leukemia or lymphoid malignancies. More particular examples of such cancers include squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, testicular cancer, esophageal cancer, tumors of the biliary tract, as well as head and neck cancer.

In another embodiment, the subject binding polypeptides are useful for the treatment of other disorders, including, without limitation, infectious diseases, autoimmune disorders, inflammatory disorders, lung diseases, neuronal or neurodegenerative diseases, liver diseases, diseases of the spine, diseases of the uterus, depressive disorders and the like. Non-limiting examples of infectious diseases include those caused by RNA viruses (e.g., orthomyxoviruses (e.g., influenza), paramyxoviruses (e.g., respiratory syncytial virus, parainfluenza virus, metapneumovirus), rhabdoviruses (e.g., rabies virus), coronaviruses, alphaviruses (e.g., Chikungunya virus) lentiviruses (e.g., HIV) and the like) or DNA viruses. Examples of infectious diseases also include, without limitation, bacterial infectious diseases, caused by, e.g., Staphylococcus aureus, Staphylococcus epidermidis, Enterococcus, Streptococcus, Escherichia coli, and other infectious diseases including, e.g., those caused by Candida albicans. Other infectious diseases include, without limitation, malaria, SARS, yellow fever, Lyme borreliosis, leishmaniasis, anthrax and meningitis. Exemplary autoimmune disorders include, but are not limited to, psoriasis, rheumatoid arthritis, Sjogren's Syndrome, graft rejection, Grave's disease, myasthenia gravis and lupus (e.g., systemic lupus erythematosus). Accordingly, this disclosure relates to a method of treating various conditions that would benefit from using a subject binding polypeptide having, e.g., enhanced half-life.

One skilled in the art would be able, by routine experimentation, to determine what an effective, non-toxic amount of modified binding polypeptide would be for the purpose of treating malignancies. For example, a therapeutically active amount of a binding polypeptide of the present disclosure may vary according to factors such as the disease stage (e.g., stage I versus stage IV), age, sex, medical complications (e.g., immunosuppressed conditions or diseases) and weight of the subject, and the ability of the modified antibody to elicit a desired response in the subject. The dosage regimen may be adjusted to provide the optimum therapeutic response. For example, several divided doses may be administered daily, or the dose may be proportionally reduced as indicated by the exigencies of the therapeutic situation.

In general, the compositions provided in the current disclosure may be used to prophylactically or therapeutically treat any neoplasm comprising an antigenic marker that allows for the targeting of the cancerous cells by the modified antibody.

Pharmaceutical Compositions and Administration Thereof

Methods of preparing and administering binding polypeptides of the current disclosure to a subject are well known to or are readily determined by those skilled in the art. The route of administration of the binding polypeptides of the current disclosure may be oral, parenteral, by inhalation or topical. The term parenteral as used herein includes intravenous, intraarterial, intraperitoneal, intramuscular, subcutaneous, rectal or vaginal administration. While all these forms of administration are clearly contemplated as being within the scope of the current disclosure, a form for administration would be a solution for injection, in particular for intravenous or intraarterial injection or drip. Usually, a suitable pharmaceutical composition for injection may comprise a buffer (e.g. acetate, phosphate or citrate buffer), a surfactant (e.g. polysorbate), optionally a stabilizer agent (e.g. human albumin), etc. In some embodiments, the binding polypeptides can be delivered directly to the site of the adverse cellular population thereby increasing the exposure of the diseased tissue to the therapeutic agent.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. In the compositions and methods of the current disclosure, pharmaceutically acceptable carriers include, but are not limited to, 0.01-0.1 M, e.g., 0.05 M phosphate buffer, or 0.8% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives may also be present such as for example, antimicrobials, antioxidants, chelating agents, and inert gases and the like. More particularly, pharmaceutical compositions suitable for injectable use include sterile aqueous solutions (where water soluble) or dispersions and sterile powders for the extemporaneous preparation of sterile injectable solutions or dispersions. In such cases, the composition must be sterile and should be fluid to the extent that easy syringability exists. It should be stable under the conditions of manufacture and storage and will typically be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants.

Prevention of the action of microorganisms can be achieved by various antibacterial and antifungal agents, for example, parabens, chlorobutanol, phenol, ascorbic acid, thimerosal and the like. In many cases, isotonic agents will be included, for example, sugars, polyalcohols, such as mannitol, sorbitol, or sodium chloride in the composition. Prolonged absorption of the injectable compositions can be brought about by including in the composition an agent which delays absorption, for example, aluminum monostearate and gelatin.

In any case, sterile injectable solutions can be prepared by incorporating an active compound (e.g., a modified binding polypeptide by itself or in combination with other active agents) in the required amount in an appropriate solvent with one or a combination of ingredients enumerated herein, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, exemplary methods of preparation include vacuum drying and freeze-drying, which yields a powder of an active ingredient plus any additional desired ingredient from a previously sterile-filtered solution thereof. The preparations for injections are processed, filled into containers such as ampoules, bags, bottles, syringes or vials, and sealed under aseptic conditions according to methods known in the art. Further, the preparations may be packaged and sold in the form of a kit. Such articles of manufacture will typically have labels or package inserts indicating that the associated compositions are useful for treating a subject suffering from, or predisposed to autoimmune or neoplastic disorders.

Effective doses of the compositions of the present disclosure, for the treatment of the above described conditions vary depending upon many different factors, including means of administration, target site, physiological state of the patient, whether the patient is human or an animal, other medications administered, and whether treatment is prophylactic or therapeutic. Usually, the patient is a human but non-human mammals including transgenic mammals can also be treated. Treatment dosages may be titrated using routine methods known to those of skill in the art to optimize safety and efficacy.

Binding polypeptides of the current disclosure can be administered on multiple occasions. Intervals between single dosages can be weekly, monthly or yearly. Intervals can also be irregular as indicated by measuring blood levels of modified binding polypeptide or antigen in the patient. In some methods, dosage is adjusted to achieve a plasma modified binding polypeptide concentration of about 1-1000 μg/ml and in some methods about 25-300 μg/ml. Alternatively, binding polypeptides can be administered as a sustained release formulation, in which case less frequent administration is required. For antibodies, dosage and frequency vary depending on the half-life of the antibody in the patient. In general, humanized antibodies show the longest half-life, followed by chimeric antibodies and nonhuman antibodies. The dosage and frequency of administration can vary depending on whether the treatment is prophylactic or therapeutic. In prophylactic applications, compositions containing the present antibodies or a cocktail thereof are administered to a patient not already in the disease state to enhance the patient's resistance. Such an amount is defined to be a “prophylactic effective dose.” In this use, the precise amounts again depend upon the patient's state of health and general immunity, but generally range from about 0.1 to about 25 mg per dose, especially about 0.5 to about 2.5 mg per dose. A relatively low dosage is administered at relatively infrequent intervals over a long period of time. Some patients continue to receive treatment for the rest of their lives. In therapeutic applications, a relatively high dosage (e.g., from about 1 to 400 mg/kg of antibody per dose, with dosages of from about 5 to 25 mg being more commonly used for radioimmunoconjugates and higher doses for cytotoxin-drug modified antibodies) at relatively short intervals is sometimes required until progression of the disease is reduced or terminated, or until the patient shows partial or complete amelioration of disease symptoms. Thereafter, the patient can be administered a prophylactic regime.

Binding polypeptides of the current disclosure can optionally be administered in combination with other agents that are effective in treating the disorder or condition in need of treatment (e.g., prophylactic or therapeutic). Effective single treatment dosages (i.e., therapeutically effective amounts) of ⁹⁰Y-labeled modified antibodies of the current disclosure range from between about 5 and about 75 mCi, such as between about 10 and about 40 mCi. Effective single treatment non-marrow ablative dosages of ¹³¹I-modified antibodies range from between about 5 and about 70 mCi, or between about 5 and about 40 mCi. Effective single treatment ablative dosages (i.e., may require autologous bone marrow transplantation) of ¹³¹I-labeled antibodies range from between about 30 and about 600 mCi, such as between about 50 and less than about 500 mCi. In conjunction with a chimeric antibody, owing to the longer circulating half-life vis-a-vis murine antibodies, an effective single treatment non-marrow ablative dosages of iodine-131 labeled chimeric antibodies range from between about 5 and about 40 mCi, such as less than about 30 mCi. Imaging criteria for, e.g., the ¹¹¹In label, are typically less than about 5 mCi.

While the binding polypeptides may be administered as described immediately above, it must be emphasized that in other embodiments a binding polypeptide may be administered to otherwise healthy patients as a first line therapy. In such embodiments the binding polypeptides may be administered to patients having normal or average red marrow reserves and/or to patients that have not, and are not, undergoing treatment. As used herein, the administration of modified antibodies or immunoadhesins in conjunction or combination with an adjunct therapy means the sequential, simultaneous, coextensive, concurrent, concomitant, or contemporaneous administration or application of the therapy and the disclosed antibodies. Those skilled in the art will appreciate that the administration or application of the various components of the combined therapeutic regimen may be timed to enhance the overall effectiveness of the treatment.

As previously discussed, the binding polypeptides of the present disclosure, immunoadhesins or recombinants thereof, may be administered in a pharmaceutically effective amount for the in vivo treatment of mammalian disorders. In this regard, it will be appreciated that the disclosed binding polypeptides will be formulated to facilitate administration and promote stability of the active agent.

A pharmaceutical composition in accordance with the present disclosure can comprise a pharmaceutically acceptable, non-toxic, sterile carrier such as physiological saline, nontoxic buffers, preservatives and the like. For the purposes of the instant application, a pharmaceutically effective amount of the binding polypeptide, immunoadhesin or recombinant thereof, conjugated or unconjugated to a therapeutic agent, shall be held to mean an amount sufficient to achieve effective binding to an antigen and to achieve a benefit, e.g., to ameliorate symptoms of a disease or disorder or to detect a substance or a cell. In the case of tumor cells, the modified binding polypeptide can interact with selected immunoreactive antigens on neoplastic or immunoreactive cells and provide for an increase in the death of those cells. Of course, the pharmaceutical compositions of the present disclosure may be administered in single or multiple doses to provide for a pharmaceutically effective amount of the modified binding polypeptide.

In keeping with the scope of the present disclosure, the binding polypeptides of the disclosure may be administered to a human or other animal in accordance with the aforementioned methods of treatment in an amount sufficient to produce a therapeutic or prophylactic effect. The binding polypeptides of the disclosure can be administered to such human or other animal in a conventional dosage form prepared by combining the antibody of the disclosure with a conventional pharmaceutically acceptable carrier or diluent according to known techniques. It will be recognized by one of skill in the art that the form and character of the pharmaceutically acceptable carrier or diluent is dictated by the amount of active ingredient with which it is to be combined, the route of administration and other well-known variables. Those skilled in the art will further appreciate that a cocktail comprising one or more species of binding polypeptides described in the current disclosure may prove to be particularly effective.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

While the present invention has been described with reference to the specific embodiments thereof, it should be understood by those skilled in the art that various changes may be made and equivalents may be substituted without departing from the true spirit and scope of the invention. It will be readily apparent to those skilled in the art that other suitable modifications and adaptations of the methods described herein may be made using suitable equivalents without departing from the scope of the embodiments disclosed herein. In addition, many modifications may be made to adapt a particular situation, material, composition of matter, process, process step or steps, to the objective, spirit and scope of the present invention. All such modifications are intended to be within the scope of the claims appended hereto. Having now described certain embodiments in detail, the same will be more clearly understood by reference to the following examples, which are included for purposes of illustration only and are not intended to be limiting.

### EXAMPLES

The present invention is further illustrated by the following examples which should not be construed as further limiting.

### Example 1: Materials and Methods

### Protein Reagents:

The following proteins were expressed and isolated: antigen with a C-terminal 8xHistidine tag; rFcRn (UniProt: P1359, p51 subunit: residues 23-298; UniProt: P07151, β2-m: residues 21-119); biotinylated cynomolgus FcRn (UniProt: Q8SPV9, p51 subunit: residues 24-297 with a C-terminal Avi-tag; UniProt: Q8SPW0, β2-m: residues 21-119); biotinylated hFcRn (UniProt: P55899, p51 subunit: residues 24-297 with a C-terminal Avi-tag; UniProt: P61769, β2-m: residues 21-119); Human CD16a (UniProt: P08637, FcγRIIIa: residues 17-208 with C-terminal HPC4 tag and valine at position 158 (V158)). The H435A and H310A/H435Q heavy chain variants were obtained from HEK293 conditioned media. mAb2 variants were cloned by Evitria and purified from suspension CHO K1 conditioned media using mAbSelect SuRe affinity columns (GE Healthcare) and buffer exchanged into phosphate buffered saline (PBS) pH 7.4 for subsequent experiments.

### Saturation Library Construction:

The WT IgG1 mAb1 antibody heavy and light chains with leader DNA sequences were incorporated into the pBH6414 and pBH6368 mammalian expression plasmids, respectively, using the NcoI and HindIII restriction enzyme sites. The saturation library was created with the Lightning Site Directed Mutagenesis Kit (Agilent) and NNK (N=A/C/G/T, K=G/T) and WWC (W=A/T) primers (IDT Technologies) to introduce all possible amino acids at the following positions: M252, 1253, S254, T256, K288, T307, K322, E380, L432, N434 and Y436 (Eu Numbering). The heavy chain DNA sequences of the three control variants in the mAb1 backbone, AAA (T307A/E380A/N434A), LS (M428L/N434S) and YTE (M252Y/S254T/T256E), were constructed into the pBH6414 vector by LakePharma.

The combination saturation library was obtained through site-directed mutagenesis of the mAb1 heavy chain with the Q5 Mutagenesis Kit (NEBiolabs) and T256D, T256E, T307Q, T307W, N434F and N434Y primers with the WT and M252Y templates in the PCR reaction. Mutation incorporation into the Ab3 backbone was performed using the Q5 Mutagenesis kit (NEBiolabs) with M252Y, T256D, T307Q and T307W primers. The creation of all of the Fc variants was confirmed through Sanger Sequencing (Genewiz, Inc.).

### Recombinant Antibody Expression and Purification:

For conditioned media screening, DNA containing the mutant heavy chain and the wild-type light chain of mAb1 were transfected into 1 mL of Expi293 mammalian cells (Invitrogen) for expression according to the manufacturer's instructions. The cells were incubated at 37° C., 5% carbon dioxide and 80% humidity with shaking at 900 rotations per minute (RPM) in a 2 mL 96 well plate (Greiner Bio-One) and sealed with an aerated membrane. The conditioned media was collected five days post-transfection and stored at −80° C. until use. The lead variants in the mAb1 and Ab3 backbones were expressed on a 30 mL scale in 125 mL flasks with 0.2 μm vented caps (Corning). The 125 mL culture flasks were shaken at 125 RPM during the entire expression duration. The conditioned media was collected five days post-transfection and filtered through 0.22 μm, 50 mL conical filters (Corning) and stored at 4° C. until purification.

Isolation of mAb1 and Ab3 was performed using 1 mL mAbSelect SuRe HiTrap columns (GE Healthcare). Following a wash step of PBS pH 7.4 for ten column volumes, the antibodies were eluted with five column volumes of 0.1 M citric acid pH 3.0 (Sigma) and neutralized with 0.5 mL of 1 M tris base pH 9.0 (Sigma). The eluted antibodies were buffer exchanged against PBS pH 7.4 and concentrated to >1 mg mL⁻¹ using 30 kDa MWCO Amicon Concentrators (Millipore) for subsequent studies. The concentration of the purified antibodies was determined from their UV absorbance at 280 nm (UV₂₈₀) with an appropriate extinction coefficient.

### Octet Conditioned Media Screening and Analysis:

Screening of conditioned media containing the mAb1 variants was performed on an Octet QK 384 with Ni-NTA biosensors (PALL Life Sciences). His-tagged antigen was captured at 15 μg mL⁻¹ for 300 sec in PBS, 0.1% Bovine Serum Albumin (BSA, Sigma) and 0.01% Tween-20 (Sigma) pH 7.4 (PBST-BSA 7.4) followed by a 20 second wash with PBST-BSA pH 7.4. The antibodies were captured for 200 sec in conditioned media diluted 1:1 with PBST-BSA pH 7.4. Following buffer wash steps in pH 6.0 buffer, FcRn binding kinetics were obtained using 200 nM rFcRn for association and dissociation times of 150 and 200 sec, respectively, at pH 6.0. At all steps during the Octet screening, the temperature was 30° C. with a shake speed of 1000 RPM. The rFcRn binding kinetic profiles were corrected to the initiation of the FcRn association phase and modeled to a 1:1 binding model using the Octet 7.1 Analysis Software.

### FcRn Binding Kinetics:

FcRn binding kinetics at pH 6.0 and pH 7.4 were measured using a Biacore T200 instrument (GE Healthcare) using modified protocols with either the direct immobilization of FcRn or the biotin CAPture kit (GE Healthcare) (see, e.g., Abdiche et al., MAbs (2015) 7:331-343; Karlsson et al., Anal. Biochem. (2016) 502:53-63). For the direct immobilization, biotinylated FcRn, at concentrations of 20 μg mL⁻¹, was immobilized for 180 s at 10 μL min⁻¹ in 10 mM sodium acetate pH 4.5 (GE Healthcare) to ˜20 RU on the surface of a C1 sensor chip through amine coupling chemistry (GE Healthcare). With the biotin CAPture kit, the CAPture reagent was captured on the CAP chip surface to a binding RU of >2,000 RU, followed by 0.1 μg mL⁻¹ FcRn in the appropriate channels for 24 s at 30 uL min⁻¹ to a final binding RU of ˜2 RU. The running buffer for the FcRn binding kinetics experiments was PBS with 0.05% Surfactant P-20 (PBS-P+, GE Healthcare) at pH 6.0 or 7.4. A concentration series of a 4-fold serial dilution from 1000 nM antibody was performed in quadruplicate for each variant, including a 0 nM control. Kinetic measurements were obtained for association and dissociation times of 180 and 300 sec, respectively at a flow rate of 10 μL min⁻¹. The C1 and CAP sensor chips were regenerated with 10 mM sodium tetraborate, 1 M NaCl pH 8.5 (GE Healthcare) for 30 sec at 50 μL min⁻¹ or 6 M guanidine hydrochloride, 250 mM sodium hydroxide (GE Healthcare) for 120 s at 50 uL min¹, respectively, followed by an additional 60-90 sec stabilization step in PBS-P+pH 6.0. Steady state RU measurements at pH 7.4 were obtained for all variants at 1000 nM in triplicate using the same C1 or CAP sensor chip and kinetic parameters as described above, except that the capture level of FcRn was increased 10- to 20-fold for both methods.

Kinetic parameters for the concentration series at pH 6.0 were fit to a bivalent model using the Biacore T200 Evaluation Software due to avidity effects. See, e.g., Suzuki et al., J. Immunol. (2010) 184: 1968-1976. Each concentration series was fit independently to obtain the average on and off-rates and binding affinities. The apparent binding affinity was calculated from the first on and off-rates from the bivalent model. The residual binding at pH 7.4 was measured using 1000 nM of each antibody in triplicate at the same time for response comparison. The steady state response of each replicate was averaged to obtain the mean and standard deviation.

### FcRn Affinity Chromatography:

In one experiment, the FcRn affinity column was created from protocols adapted from Schlothauer et al. 2013, mAbs 5: 576-586. A 1 mL Streptavidin HP HiTrap column (GE Healthcare) was equilibrated with binding buffer (20 mM sodium phosphate (Sigma) pH 7.4, 150 mM sodium chloride (NaCl; Sigma)) at 1 mL min⁻¹ for five column volumes followed by an injection of 4 milligram of biotinylated cynoFcRn. The column was washed with binding buffer and stored at 4° C. until use.

The FcRn affinity column was equilibrated with low pH buffer (20 mM 2-(N-morpholino)ethanesulfonic acid (MES; Sigma) pH 5.5; 150 mM NaCl) for five column volumes prior to injection with 300 μg of each antibody. The pHs of the antibody solutions were adjusted to pH 5.5 with low pH buffer. Following a ten column volume wash with low pH buffer, the antibodies were eluted by a linear pH gradient with high pH buffer (20 mM 1,3-bis(tris(hydroxymethyl)methylamino)propane (bis tris propane; Sigma) pH 9.5; 150 mM NaCl) over 30 column volumes at 1 mL min⁻¹ in 1 mL fractions and monitoring the UV₂₈₀. The FcRn affinity column was re-equilibrated with ten column volumes of low pH buffer for subsequent runs or binding buffer for storage. All variants were performed in triplicate.

The FcRn affinity column elution profile for each variant was modeled to a single Gaussian distribution using Equation 1 in Sigmaplot 11 (Systat Software, Inc.) to determine the elution volume at the UV₂₈₀ maximum.

$${UV}_{280} = {y}_{0} + a * {exp}^{\frac{- {( x - {x}_{0} )}^{2}}{2 b}} \qquad ( Equation 1 )$$

Where x₀ is the elution volume at the UV₂₈₀ peak maximum, y₀ is the baseline UV₂₈₀ absorbance and a and b are related to the full width at half max of the distribution. The pH of each fraction was measured by a Corning Pinnacle 540 pH meter and correlated to the elution volume using a linear regression.

In another experiment, The FcRn affinity column was adapted from Schlothauer et al. 2013, mAbs 5: 576-586 with biotinylated hFcRn on a 1 mL Streptavidin HP HiTrap column (GE Healthcare). The column was injected with 300 ug of each antibody in low pH buffer (20 mM 2-(N-morpholino)ethanesulfonic acid (MES; Sigma) pH 5.5; 150 mM NaCl) on an AKTA Pure System (AKTA). The antibodies were eluted by a linear pH gradient created with low and high pH buffer (20 mM 1,3-bis(tris(hydroxymethyl)methylamino)propane (bis tris propane; Sigma) pH 9.5; 150 mM NaCl) over 30 column volumes at 0.5 mL min⁻¹ and monitoring the absorbance and pH. The column was re-equilibrated with low pH buffer for subsequent runs. All variants were performed in triplicate. The FcRn affinity column elution profiles were fit to a single Gaussian distribution in Sigmaplot 11 (Systat Software, Inc.) to determine the elution volume and pH from at the UV₂₈₀ maximum.

### Differential Scanning Fluorimetry:

The Differential Scanning Fluorimetry (DSF) experiments were performed on a BioRad CFX96 real time system thermal cycler (BioRad) on 20 μL reactions. The antibody samples and 5000× stock of Sypro Orange dye (Invitrogen) were diluted to 0.4 mg mL⁻¹ and 10×, respectively, in PBS pH 7.4. The antibodies and Sypro Orange were mixed in a 1:1 ratio in 96-well PCR plates and sealed with adhesive microseal (BioRad) to final concentrations of 0.2 mg mL⁻¹ of each antibody and 5× Sypro Orange dye. All antibody variants were performed in triplicate. The thermal cycler program consisted of a 2 minute equilibration step at 20° C. followed by constant temperature ramping rate of 0.5° C./5 sec to a final temperature of 100° C. Fluorescence measurements of each well were acquired using the FAM excitation wavelength (485 nm) and ROX emission (625 nm) detectors suitable for Sypro orange fluorescence (see, e.g., Biggar et al. 2012, Biotechniques 53: 231-238). The DSF fluorescence intensity profile and first derivative were exported from the BioRad CFX Manager and analyzed in Sigmaplot 11. The Tₘ was defined as the midpoint of the first transition in the fluorescence intensity profile.

### FcγRIIIa Binding Kinetics:

Binding kinetics and affinity were measured using a Biacore T200 instrument (GE Healthcare) (Zhou et al. 2008 Biotechnol. Bioeng. 99: 652-665). Anti-HPC4 antibody (Roche), at 50 μg mL⁻¹ in Acetate pH 4.5, was coupled to the surface of CM5 sensor chip for 600 sec at 10 μl min⁻¹ with amine chemistry to a final density of >20,000 RU. The running buffer for the FcγRIIIa binding kinetics experiments was HEPES Buffered Saline with 0.05% Surfactant P-20 (HBS-P+, GE Healthcare) and 2 mM Calcium Chloride (CaCl₂, Fluka) at pH 7.4. Each kinetic trace was initialized with the capture of 1.25 μg mL⁻¹ HPC4-tagged FcγRIIIa-V158 for 30 sec at 5 μl min¹. Association and dissociation kinetics at 300 nM of each variant were measured for each variant for 120-180 sec for each step at 5 μl min-1. Upon completion of the kinetic measurements, the CM5 chip was regenerated with HBS-P+ buffer supplemented with 10 mM EDTA (Ambion). Prior to the next kinetic measurements, the CM5 chip was washed for 120 sec with HBS-P+ with CaCl₂.

In one experiment, he FcγRIIIa kinetic experiments were analyzed in a similar manner as described for the FcRn binding at pH 7.4. For the WT, benchmark, lead single and combination variants, kinetics at a series of a 3-fold serial dilution from 1000 nM were obtained to determine the binding affinity to FcγRIIIa. The steady state RU at each concentration and replicate were determined, plotted as a function of antibody concentration and fit to a steady state model as shown in Equation 2.

$$RU = offset + \frac{( {R}_{max} - offset ) * [ Antibody ]}{[ Antibody ] + {K}_{D , app}} \qquad ( Equation 2 )$$

Where offset is the baseline RU at 0 nM antibody, Rₘₐₓ is the plateau RU at high antibody concentrations, [Antibody] is the concentration of antibody and K_{D,app} is the apparent binding affinity of the interaction between the variants and FcγRIIIa.

In another experiment, the FcγRIIIa kinetic experiments were analyzed in a similar manner as described for the FcRn binding at pH 7.4 using the average steady state binding response. For all variants, the steady state RU of 300 nM antibody was determined in triplicate and averaged. The fold change in response change relative to WT (Response Fold Change) was determined for comparison between the variants in each backbone.

### Isoelectric Focusing

The isoelectric point (p1) of the lead variants was determined using capillary electrophoresis on a Maurice C (Protein Simple). Each 200 μL sample contained 0.35% methyl cellulose (Protein Simple), 4% pharmalyte 3-10 (GE Healthcare), 10 mM arginine (Protein Simple), 0.2 mg mL⁻¹ antibody and the 4.05 and 9.99 μl markers (Protein Simple). The sample was loaded into the capillary for 1 min at 1500 V, followed by a separation phase for 6 min at 3000 V and monitored using tryptophan fluorescence. The μl for each variant was determined using the Maurice C software and defined as the pH at the fluorescence maximum for the major species.

### Homogeneous Bridging Rheumatoid Factor (RF) ELISA

Antibodies were biotinylated and digoxigen-labeled using the EZ-Link Sulfo-NHS-LC-Biotin and Mix-n-Stain™ Digoxigenin Antibody Labeling Kits (Biotium) according to the manufacturer's instructions. A stock solution containing 4 μg mL⁻¹ of the biotinylated and digoxigenin-labeled antibodies was prepared for each variant and mixed in a 1:1 ratio with 300 U/mL RF (Abcam). Following incubation at room temperature for 20 hours, 100 μL of each antibody-RF mixture was added to Streptawell plates (Sigma-Aldrich) and incubated at room temperature for 2 hours. The plate was washed three times with PBS pH 7.4 with 0.05% Tween-20 and 100 μL of a 1:2000 dilution of HRP-conjugated anti-digoxigenin secondary antibody (Abcam) was added to each well. After a 2-hour incubation at room temperature, the wells were washed and treated with 100 μL of the TMB substrate (Abcam) for 15 minutes at room temperature. The reaction was stopped with 100 μL of the stop solution (Abcam) and the absorbance was measured at 450 nm on a SpectraMax plate reader. A well containing no antibody-RF mixture provided the blank subtraction and the experiment was repeated three times. P-values were determined using the student's t-test.

### In Vivo Pharmacokinetics

Pharmacokinetics studies were conducted in cynomolgus monkey and hFcRn transgenic mouse (Tg32 strain, Jackson Laboratory, Bar Harbor, Me.). In monkey studies WT, LS, DQ, DW and YD variants in the mAb2 backbone were administered as a single intravenous dose of 2.5 mg/kg into the brachiocephalic vein with a dose volume of 1.5 mL/kg, to three treatment-naive male cynomolgus. Blood samples (0.5 mL) were collected by venipuncture of saphenous vein at 8 sampling times post dosing: 0.0035, 0.17, 1, 3, 7, 14, 21 and 28 days. Once collected, blood samples were centrifuged at 4° C. for 10 minutes at 1500 g and stored at −80° C.

In hFcRn mice the antibody variants were administered as single intravenous dose of 2.5 mg/kg into the tail vein with a dose volume of 5 ml/kg. At each time point 20 μl blood was collected from saphenous vein using prefilled heparin capillaries. Collected blood samples were transferred into microtubes and centrifuged at 1500 g for 10 minutes and at 4° C. Plasma samples were collected, pooled for each time point (6 mice/sample), and stored at −80° C. prior to analysis.

All in vivo studies were conducted in compliance with the Sanofi institutional animal care policy. The monkey and mouse studies were approved by French “Ministère de l′Enseignement Superieur et de la Recherche” and German “Regierungspraesidium Darmstadt.” The concentration of each mAb2 variant at each time point was determined by a bottom-up LC-MS/MS assay. After a precipitation of a plasma aliquot, the plasma pellet was subjected to a protein denaturation, reduction, alkylation, trypsin digestion and solid phase extraction prior to analysis of surrogate peptides. Calibration standards were prepared by spiking the mAb2 variant into the plasma at 1.00, 2.00, 5.00, 10.0, 20.0, 50.0, 100, 200 and 400 μg mL⁻¹. Peptide separation was performed on a Waters Acquity UPLC system with a reverse phase XBridge BEH C18 column (2.1×150 mm, 3.5 μM, 300 Å, Waters) at a flow rate of 300 μL min⁻¹ in a step-wise gradient of 0.1% formic acid in water and 0.1% formic acid in acetonitrile. For detection, a Sciex AP15500 mass spectrometer was used in positive ion mode, with the source temperature at 700° C., the ionspray voltage at 5500 V, curtain and nebulizer gases at 40 and the collision gas at mid. The dwell times were 20 ms and the entrance potential was at 10 V for each transition. The multiple reaction monitoring transitions for two unique surrogate peptides of the mAb2 backbone were used for concentration determination relative to the standards and controls using the peak area from the MQIII integration algorithm of the Analyst software. The clearance rate and serum half-life were obtained from a non-compartmental model of the antibody concentration as a function of time using the Phoenix Software (Certara). All time points showing a sharp reduction in concentration were excluded from the mean plasma concentration due to, without being bound by any theory, presumed target-mediated drug disposition (TMDD) and/or anti-drug antibody (ADA) interference.

### Example 2: Octet Screening of Saturation Point Mutations in Conditioned Media

FcRn is a heterodimer of an MHC class-I-like α-domain and a β2-macroglobulin (β2-m) subunit (FIG. 1A), common to a majority of the Fc receptors, and recognizes regions on the antibody Fc heavy chain distinct from the other FcγRs (see, e.g., Oganesyan et al. 2014 J. Biol. Chem. 289: 7812-7824; and Shields et al. 2001 supra).

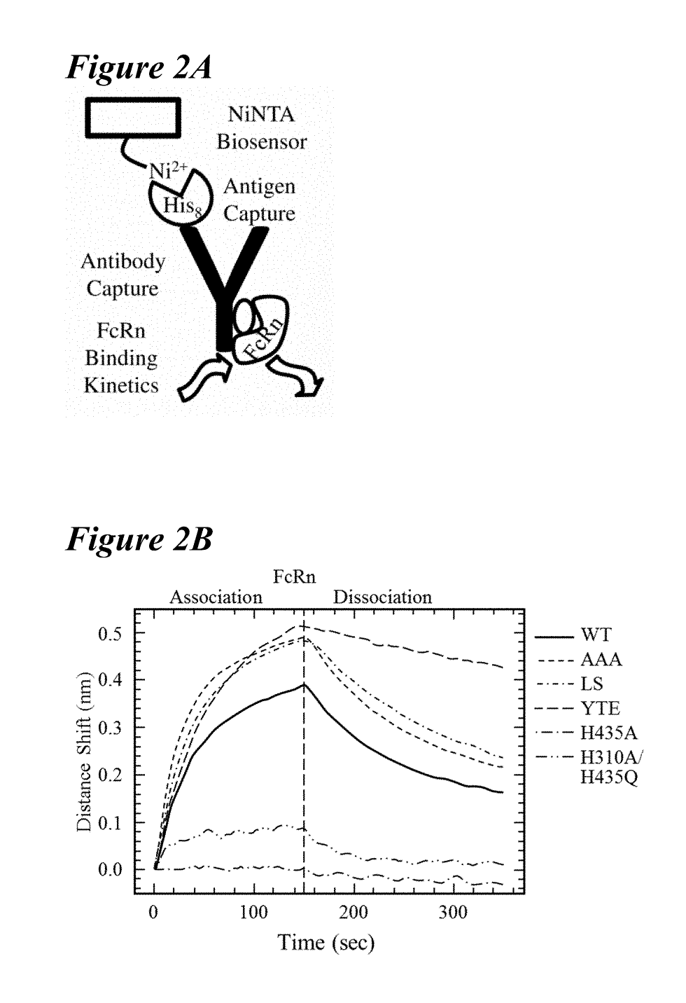

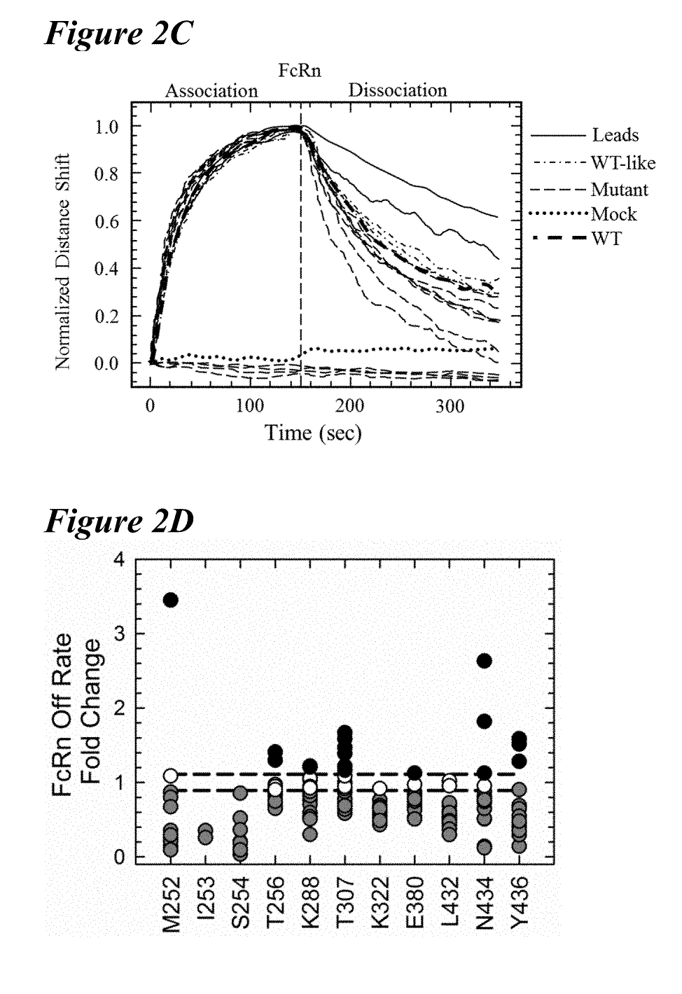

In order to identify variants with slower FcRn off-rates than the WT antibody, a biolayer interferometry (BLI)-based assay was designed to screen the antibody variants in conditioned media in a high throughput manner (FIG. 2A). This assay was developed using several benchmark variants which enhance (AAA, LS and YTE) or reduce (H435A, H310A/H435Q) the affinity for FcRn at pH 6.0, in comparison to the WT antibody. NiNTA biosensors captured the his-tagged antigen and, subsequently, each antibody variant at pH 7.4 to mimic conditioned media (FIG. 2A). Binding kinetics to rat FcRn (rFcRn) at pH 6.0, which has a ˜25-fold slower off-rate from human IgG1 and is more amenable for Octet studies than human FcRn (hFcRn), were measured for each of the six variants (FIG. 2B). The H435A (FIG. 2B, long dashes interspersed with single dot) and H310A/H435Q (FIG. 2B, long dashes interspersed with two dots) variants show little to no FcRn binding kinetics (also see, e.g., Shields et al. 2001 supra; Medesan et al. 1997 supra; and Raghavan et al. 1995 supra). The AAA (FIG. 2B, short dashes), LS (FIG. 2B, short dashes interspersed with single dot) and YTE (FIG. 2B, long dashes) variants all display slower dissociation kinetics compared to the WT (FIG. 2B, solid line) with between a 2-7.3-fold reduction in the FcRn off-rate. This demonstrated that Octet screening is suitable to distinguish between variants with perturbed rFcRn dissociation kinetics.

An IgG1 antibody, mAb1, served as a model system to create a saturation mutagenesis library to screen for mutants with a reduced FcRn off-rate. Eleven positions in the Fc region of mAb1 were selected based on their proximity or direct contribution to the FcRn interface (FIGS. 1A and 1B) (see, e.g., Oganesyan et al. 2014 supra; and Shields et al. 2001 supra). All point mutations at these positions were constructed using site directed mutagenesis and transfected in Expi293 cells for expression. Conditioned media screening was performed for the saturation library mutants as described above. The normalized FcRn binding Octet sensorgrams for a subset of the variants are shown in FIG. 2C (long dashes) with the wild-type (FIG. 2C, thick long dashes) and mock-negative control (FIG. 2C, dotted line). The mock showed a lack of observable FcRn binding. Several mutants clearly disrupted the binding of rFcRn as little to no signal change was observed in the kinetic profiles (FIG. 2C, long dashes, located below the dotted line (mock)). The cutoff for variants with improved FcRn off-rate was defined as three standard deviations lower than the mean of the WT antibody. In the subset of mutations shown in FIG. 2C, two (FIG. 2C, solid lines) had significantly reduced off-rates compared to the wild-type antibody (FIG. 2C, thick long dashes), while the remaining variants had similar (FIG. 2C, short dashes interspersed by single dot) or faster (FIG. 2C, long dashes above dotted line (mock)) rFcRn off-rates.

The rFcRn off-rates for all of the single point mutations are shown in FIG. 2D and FIG. 14 by position and mutation. In FIG. 14, the data is sorted into one of four categories depending on the fold change of rFcRn off-rate compared to wild-type, and wildtype species are indicated by black squares.

In FIG. 14, the fold-change in the rFcRn off-rate for all possible substitutions at the eleven positions of the saturation library were normalized to the average of the WT antibody and color coded. All mutants fell into one of four categories: little to no binding (dark gray), faster rFcRn off-rate (gray), WT-like rFcRn off-rate (horizontal lines) and slower rFcRn off-rate (grids). Multiple variants possessed slower rFcRn off-rates than the WT antibody (grids).

Mutants colored in dark gray in FIG. 14 showed little to no binding to rFcRn in a similar manner as the mock (FIG. 2C, dotted line), and localized to the M252, I253 and S254 loop. The only mutations at I253 were methionine and valine, and both significantly increased the rFcRn off-rate, further supporting the importance of I253 to the FcRn interaction. Another 120 variants (FIG. 2D and FIG. 14, light gray rectangles) destabilized the interaction with rFcRn with approximately 50% located in each the C_{H}2 and C_{H}3 domains. Twenty-five mutants have a WT-like off-rate (FIG. 2D and FIG. 14, white rectangles) with eight of the 11 position possessing at least one WT-like mutation (FIG. 14, white rectangles). The following mutations had a significantly reduced rFcRn off-rate compared to wild-type (FIG. 2D and FIG. 14, black rectangles): M252Y, T256D/E, K288D/N, T307A/E/F/M/Q/W, E380C, N434F/P/Y and Y436H/N/W. The M252Y, N434F and N434Y mutations possessed off-rates greater than two-fold slower than the WT antibody (FIG. 2D). These mutations were expressed and purified with protein A chromatography for further in vitro FcRn kinetic characterization.

### Example 3: Biacore FcRn Binding Kinetics at pH 6.0

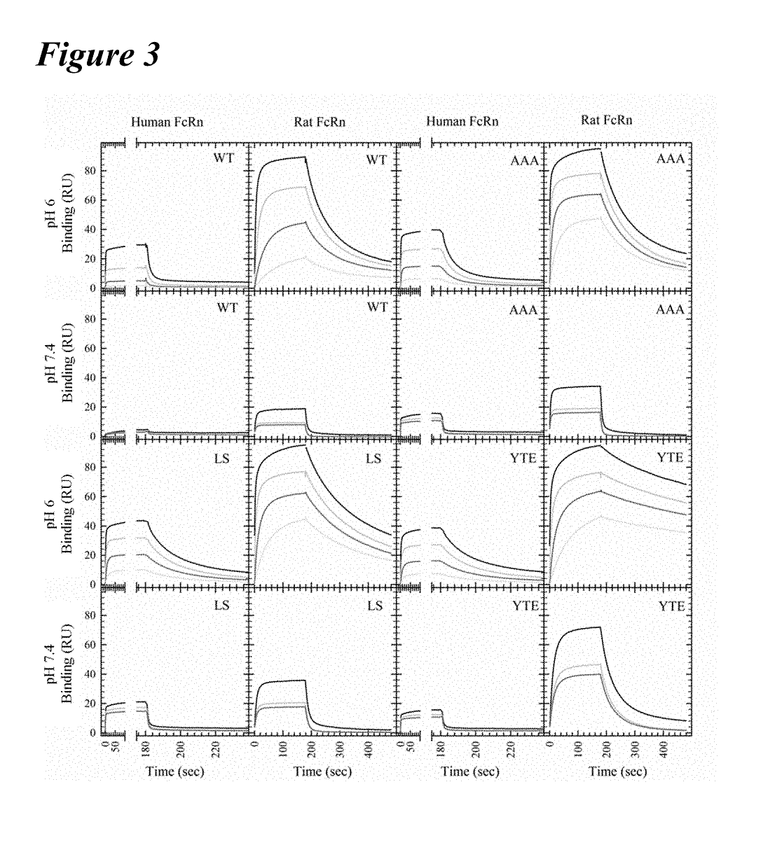

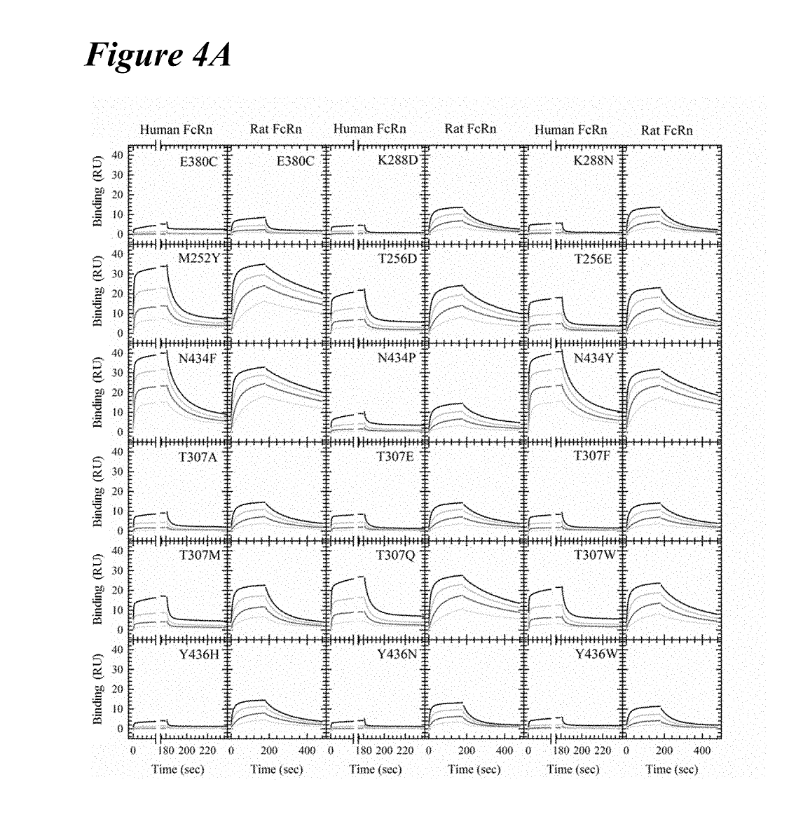

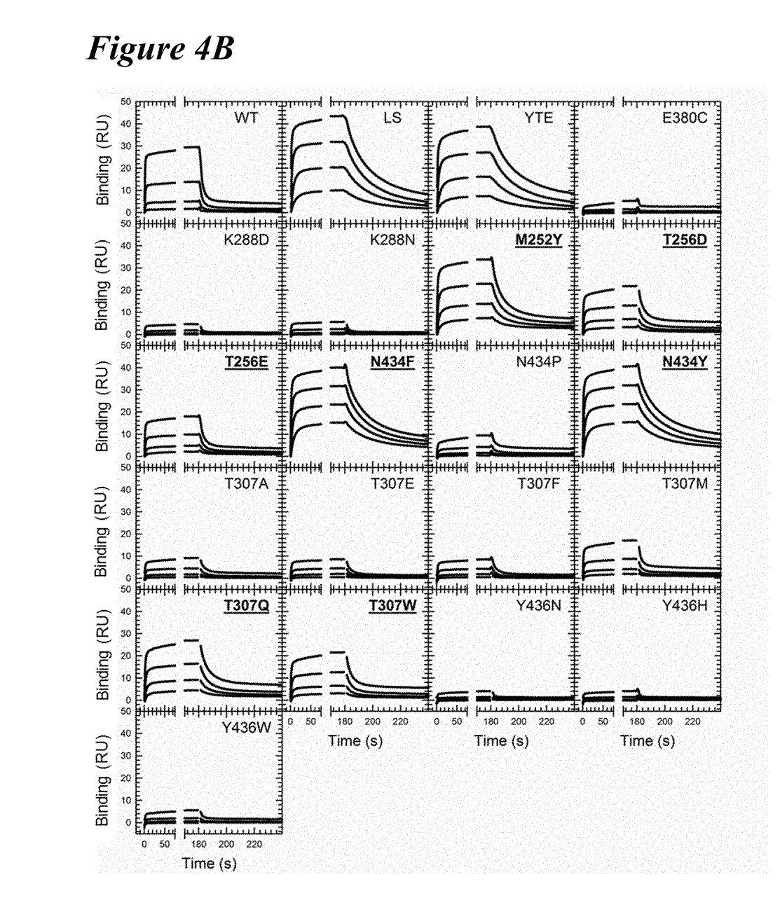

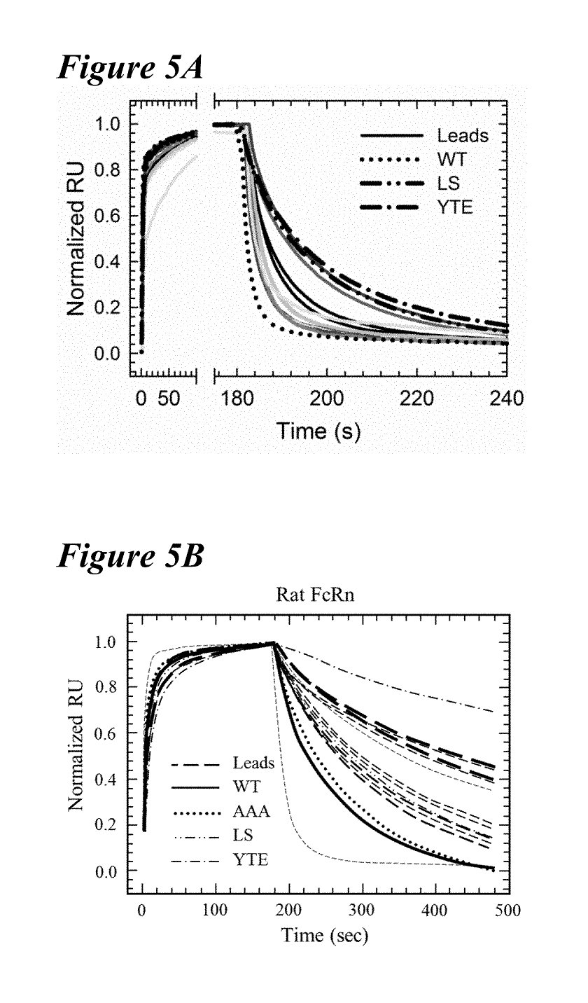

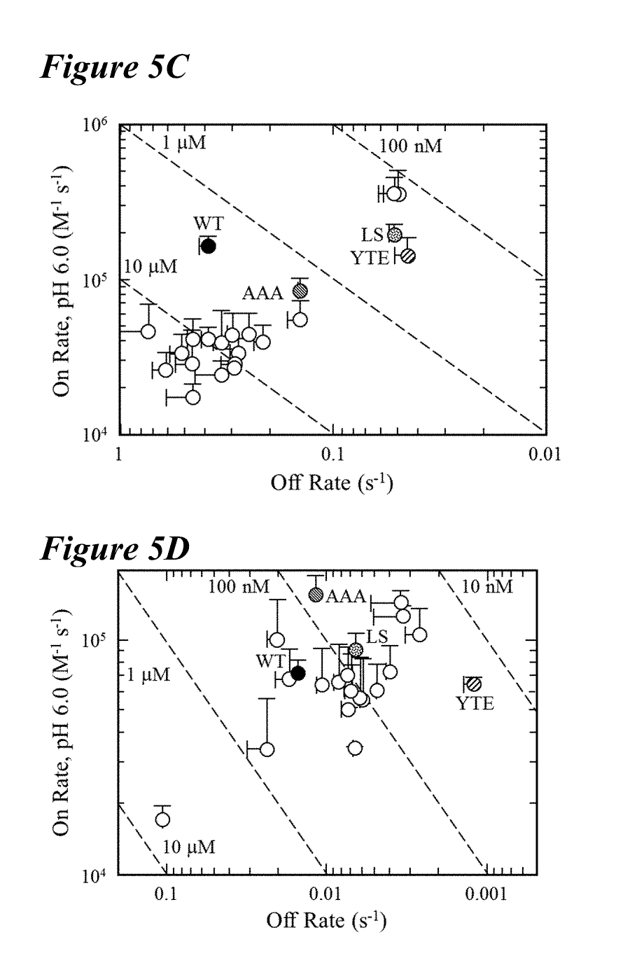

The AAA, LS and YTE variants served as positive controls in the FcRn binding kinetics measurements using Biacore to both human and rat FcRn at pH 6.0. Concentration-dependent binding to FcRn was observed for all variants, including the wild-type, benchmark (FIG. 3) and leads (FIGS. 4A and 4B), and the binding profile of a single injection with human and rat FcRn are shown in FIGS. 5A and 5B, respectively. The wild-type antibody had binding affinity for human and rat FcRn of 2380±470 nM and 207±43 nM affinities, respectively (Table 1).

TABLE 1
In vitro Characterization Parameters of Purified Lead Antibodies for mAb1.

| | Octet | FcRn | | | | |
|---|---|---|---|---|---|---|
| | pH 6.0 | Affinity | | Biacore pH 6.0 | | |
| | rFcRn | Column | DSF | hFcRn | | |
| | Off- | Elution | Tₘ | On- | Off- | |
| Mutant | rate | pH | (° C.) | rate | rate | K_{D,app} |
| E380C | 2.08 ± 0.18 | 7.18 ± 0.11 | 64.7 ± 0.5 | 1.73 ± 0.39 | 4.57 ± 1.50 | >10,000 |
| K288D | 3.79 ± 0.06 | 7.33 ± 0.10 | 65.8 ± 0.1 | 3.31 ± 1.10 | 5.13 ± 0.75 | >10,000 |
| K288N | 4.11 ± 0.08 | 7.39 ± 0.02 | 66.7 ± 0.3 | 4.12 ± 1.42 | 4.54 ± 0.34 | >10,000 |
| M252Y | 0.95 ± 0.03 | 7.88 ± 0.03 | 64.4 ± 0.2 | 5.50 ± 1.83 | 1.43 ± 0.23 | 3100 ± 1500 |
| N434F | 1.18 ± 0.05 | 8.30 ± 0.05 | 67.8 ± 0.2 | 35.4 ± 15.3 | 0.50 ± 0.08 | 165 ± 73 |
| N434P | 3.80 ± 0.08 | 7.56 ± 0.02 | 63.6 ± 0.5 | 2.42 ± 0.54 | 3.35 ± 1.10 | >10,000 |
| N434Y | 1.33 ± 0.04 | 8.46 ± 0.02 | 67.3 ± 0.5 | 35.9 ± 9.6 | 0.52 ± 0.10 | 137 ± 33 |
| T256D | 2.24 ± 0.03 | 7.82 ± 0.07 | 64.7 ± 0.2 | 4.41 ± 1.72 | 2.51 ± 0.65 | 6700 ± 3540 |
| T256E | 3.26 ± 0.04 | 7.63 ± 0.06 | 66.3 ± 0.6 | 3.90 ± 2.37 | 3.38 ± 0.28 | >10,000 |
| T307A | 2.98 ± 0.06 | 7.61 ± 0.03 | 68.0 ± 0.4 | 2.85 ± 0.72 | 2.91 ± 0.46 | >10,000 |
| T307E | 3.29 ± 0.08 | 7.58 ± 0.03 | 70.2 ± 0.5 | 4.37 ± 1.63 | 2.98 ± 0.37 | 8130 ± 5070 |
| T307F | 2.80 ± 0.07 | 7.61 ± 0.03 | 70.2 ± 0.3 | 2.70 ± 0.83 | 2.92 ± 0.13 | >10,000 |
| T307M | 3.47 ± 0.13 | 7.40 ± 0.08 | 70.0 ± 0.4 | 4.08 ± 0.83 | 3.87 ± 0.28 | >10,000 |
| T307Q | 1.84 ± 0.05 | 7.86 ± 0.06 | 70.3 ± 0.6 | 3.96 ± 1.10 | 2.15 ± 0.22 | 5720 ± 1530 |
| T307W | 2.42 ± 0.08 | 7.75 ± 0.07 | 63.0 ± 0.5 | 3.33 ± 0.83 | 2.77 ± 0.29 | 8740 ± 2440 |
| Y436H | 3.22 ± 0.08 | 7.33 ± 0.05 | 68.7 ± 0.3 | 2.59 ± 0.78 | 6.06 ± 1.00 | >10,000 |
| Y436N | 5.25 ± 0.22 | 7.22 ± 0.05 | 65.8 ± 0.5 | 4.60 ± 2.36 | 7.37 ± 3.34 | >10,000 |
| Y436W | 5.18 ± 0.18 | 7.39 ± 0.03 | 68.6 ± 0.7 | 2.84 ± 1.90 | 4.62 ± 0.92 | >10,000 |
| WT | 5.01 ± 0.45 | 7.37 ± 0.05 | 69.0 ± 0.2 | 16.2 ± 2.9 | 3.86 ± 0.38 | 2380 ± 470 |
| AAA | 3.77 ± 1.03 | 7.94 ± 0.06 | 61.3 ± 0.6 | 8.37 ± 1.82 | 1.44 ± 0.04 | 1780 ± 380 |
| LS | 3.38 ± 0.23 | 8.29 ± 0.03 | 68.5 ± 0.3 | 19.3 ± 3.5 | 0.52 ± 0.03 | 272 ± 40 |
| YTE | 0.66 ± 0.17 | 8.14 ± 0.03 | 61.2 ± 0.3 | 14.3 ± 4.4 | 0.45 ± 0.07 | 342 ± 117 |

| | | | | Biacore pH 7.4 | |
|---|---|---|---|---|---|
| | Biacore pH 6.0 | | | hFcRn | rFcRn |
| | rFcRn | | | Steady | Steady |
| | On- | Off- | | State | State |
| Mutant | rate | rate | K_{D,app} | RU | RU |
| E380C | 1.71 ± 0.25 | 106 ± 1 | 6310 ± 880 | 5.7 ± 0.1 | 27.7 ± 4.0 |
| K288D | 6.61 ± 3.01 | 8.43 ± 0.60 | 149 ± 63 | 3.8 ± 0.2 | 21.9 ± 3.1 |
| K288N | 6.42 ± 2.80 | 10.7 ± 0.9 | 190 ± 73 | 3.9 ± 0.2 | 20.4 ± 2.7 |
| M252Y | 10.6 ± 3.1 | 2.64 ± 0.62 | 25 ± 3 | 8.6 ± 0.9 | 57.7 ± 10.7 |
| N434F | 12.6 ± 1.4 | 3.36 ± 1.79 | 26 ± 13 | 20.1 ± 3.4 | 54.9 ± 9.1 |
| N434P | 3.44 ± 0.07 | 6.67 ± 0.18 | 194 ± 9 | 3.5 ± 0.4 | 4.3 ± 0.6 |
| N434Y | 14.5 ± 1.82 | 3.46 ± 1.86 | 23 ± 12 | 22.6 ± 4.2 | 47.0 ± 6.3 |
| T256D | 6.09 ± 1.84 | 4.84 ± 0.08 | 86 ± 27 | 5.8 ± 0.6 | 30.4 ± 3.4 |
| T256E | 6.29 ± 1.59 | 6.94 ± 0.97 | 113 ± 15 | 4.8 ± 0.5 | 23.0 ± 2.7 |
| T307A | 6.09 ± 2.72 | 7.08 ± 0.67 | 132 ± 48 | 5.2 ± 0.6 | 23.1 ± 2.8 |
| T307E | 5.55 ± 2.82 | 6.03 ± 0.20 | 141 ± 65 | 5.7 ± 0.7 | 21.5 ± 2.6 |
| T307F | 5.69 ± 2.83 | 6.15 ± 0.14 | 131 ± 63 | 4.9 ± 0.7 | 21.9 ± 2.7 |
| T307M | 6.81 ± 2.34 | 17.1 ± 4.0 | 279 ± 140 | 4.4 ± 0.7 | 15.3 ± 1.9 |
| T307Q | 7.35 ± 2.15 | 4.04 ± 0.28 | 58 ± 16 | 6.4 ± 0.8 | 24.3 ± 3.0 |
| T307W | 7.11 ± 2.29 | 7.37 ± 0.41 | 111 ± 32 | 5.8 ± 0.7 | 19.2 ± 3.3 |
| Y436H | 5.06 ± 0.13 | 7.30 ± 0.88 | 131 ± 9 | 3.6 ± 0.5 | 16.3 ± 1.8 |
| Y436N | 10.1 ± 4.9 | 20.3 ± 3.1 | 233 ± 86 | 3.6 ± 0.4 | 17.4 ± 2.0 |
| Y436W | 3.44 ± 2.18 | 23.7 ± 7.7 | 1140 ± 950 | 3.7 ± 0.4 | 10.3 ± 1.2 |
| WT | 7.26 ± 1.01 | 15.2 ± 0.23 | 207 ± 43 | 4.3 ± 1.0 | 12.2 ± 0.5 |
| AAA | 15.7 ± 3.3 | 11.7 ± 1.1 | 77 ± 18 | 13.9 ± 3.1 | 23.6 ± 4.9 |
| LS | 9.08 ± 1.58 | 6.58 ± 0.39 | 74 ± 9 | 18.3 ± 4.6 | 24.8 ± 4.8 |
| YTE | 6.52 ± 0.46 | 1.21 ± 0.21 | 18 ± 2 | 13.2 ± 3.5 | 53.9 ± 1.2 |

All data shown in Table 1 was obtained using the experimental techniques shown at the top of each column.

The rFcRn off-rate by Octet using purified proteins was measured as a comparison to the kinetic constants obtained from the screening in conditioned media. The elution pH was determined by FcRn affinity chromatography in triplicate (n=3) and DSF probed the thermal stability in triplicate (n=3). FcRn binding kinetics to human and rat FcRn were obtained from Biacore with a series of antibody concentrations in duplicate (n=2) and fit independently. The steady state binding response (RU) of each variant with human and rat FcRn at pH 7.4 was measured with 1000 nM antibody in triplicate (n=3) using Biacore. Units for each measurement are as follows: Octet pH 6.0 rFcRn Off-rate (×10⁻³ s⁻¹); Elution pH (unit-less); DSF Tₘ (° C.); Biacore pH 6.0 hFcRn On-rate (×10⁴ M⁻¹ s⁻¹), Off-rate (×10⁻¹ s⁻¹) and K_{D,app} (×10⁹ M); Biacore pH 6.0 rFcRn On-rate (×10⁴ M⁻¹ s⁻¹), Off-rate (×10⁻³ s⁻¹) and K_{D,app} (×10⁹ M); and Biacore pH 7.4 Steady State Binding Response (RU).

In FIG. 5B, the AAA (dotted), LS (dashes interspersed by two dots) and YTE (dashes interspersed by single dot) variants had between a 1.6 and 10.4-fold enhanced binding affinity compared to WT. The identity of the benchmark variant with the tightest FcRn affinity was species specific as LS had the tightest affinity to hFcRn, while rFcRn had a tighter affinity for YTE (Table 2A).

TABLE 2A
In vitro Characterization Parameters of Purified Double Combination Antibodies for
mAb1.

| | FcRn | | | | | | | | | |
|---|---|---|---|---|---|---|---|---|---|---|
| | Affinity | | Biacore pH 6.0 | | | | | | Biacore pH 7.4 | |
| | Column | | hFcRn | | | rFcRn | | | hFcRn | rFcRn |
| | Elution | DSF | On- | Off- | | On- | Off- | | Steady | Steady |
| Variant | pH | Tₘ (° C.) | rate | rate | K_{D,app} | rate | rate | K_{D,app} | State RU | State RU |
| MDQN | 7.92 ± 0.06 | 67.9 ± 0.4 | 3.76 ± 0.38 | 8.72 ± 0.10 | 232 ± 24 | 1.33 ± 0.22 | 1.27 ± 0.07 | 9.54 ± 1.66 | 10.7 ± 1.0 | 39.1 ± 4.5 |
| MDTF | 8.41 ± 0.07 | 62.3 ± 0.2 | 9.68 ± 0.64 | 2.90 ± 0.04 | 29.9 ± 2.0 | 2.18 ± 0.20 | 0.39 ± 0.01 | 1.78 ± 0.17 | 29.2 ± 4.0 | 63.0 ± 6.0 |
| MDTY | 8.45 ± 0.04 | 61.5 ± 0.2 | 18.3 ± 0.6 | 2.85 ± 0.01 | 15.6 ± 0.5 | 4.13 ± 0.15 | 0.25 ± 0.35 | 0.60 ± 0.85 | 37.1 ± 5.0 | 71.6 ± 6.6 |
| MDWN | 7.92 ± 0.04 | 57.8 ± 0.4 | 5.29 ± 0.14 | 8.92 ± 0.32 | 169 ± 8 | 1.57 ± 0.12 | 1.29 ± 0.01 | 8.24 ± 0.85 | 12.2 ± 1.3 | 42.1 ± 4.7 |
| MEQN | 7.84 ± 0.06 | 68.0 ± 0.5 | 2.87 ± 0.01 | 14.3 ± 0.2 | 499 ± 6 | 1.46 ± 0.25 | 1.56 ± 0.94 | 10.7 ± 6.7 | 5.8 ± 0.6 | 23.4 ± 3.1 |
| METF | 8.23 ± 0.03 | 64.1 ± 0.7 | 5.36 ± 1.41 | 4.19 ± 0.03 | 78.2 ± 20.5 | 1.08 ± 0.11 | 0.81 ± 0.07 | 7.53 ± 1.03 | 23.3 ± 3.2 | 59.0 ± 5.6 |
| METY | 8.38 ± 0.04 | 63.5 ± 0.6 | 6.28 ± 1.62 | 3.93 ± 0.02 | 62.6 ± 16.2 | 1.27 ± 0.11 | 0.98 ± 0.07 | 7.71 ± 0.84 | 26.8 ± 3.7 | 62.2 ± 6.3 |
| MEWN | 7.78 ± 0.04 | 58.2 ± 0.4 | 4.32 ± 0.36 | 14.0 ± 0.1 | 323 ± 27 | 1.81 ± 0.01 | 1.92 ± 0.05 | 10.6 ± 0.26 | 7.7 ± 0.9 | 33.0 ± 4.2 |
| MTQF | 8.56 ± 0.14 | 69.3 ± 0.2 | 5.74 ± 1.05 | 2.46 ± 0.05 | 42.9 ± 7.9 | 1.04 ± 0.07 | 0.40 ± 0.01 | 3.89 ± 0.26 | 34.2 ± 4.6 | 62.5 ± 7.6 |
| MTQY | 8.68 ± 0.15 | 69.2 ± 0.2 | 6.22 ± 1.38 | 2.02 ± 0.06 | 32.4 ± 7.3 | 1.16 ± 0.06 | 0.48 ± 0.01 | 4.11 ± 0.23 | 38.4 ± 5.2 | 63.0 ± 7.7 |
| MTWF | 8.61 ± 0.06 | 60.9 ± 0.2 | 7.87 ± 0.31 | 3.01 ± 0.08 | 38.2 ± 1.8 | 2.11 ± 0.06 | 0.57 ± 0.01 | 2.70 ± 0.08 | 30.5 ± 4.2 | 65.1 ± 6.4 |
| MTWY | 8.62 ± 0.14 | 62.1 ± 0.5 | 14.8 ± 0.8 | 3.17 ± 0.03 | 21.5 ± 1.2 | 4.80 ± 0.07 | 0.30 ± 0.04 | 0.62 ± 0.08 | 37.2 ± 4.9 | 69.6 ± 7.0 |
| YDTN | 8.29 ± 0.06 | 59.6 ± 0.9 | 6.33 ± 1.23 | 5.93 ± 0.20 | 93.6 ± 18.4 | 2.85 ± 0.11 | 1.67 ± 0.16 | 5.86 ± 0.61 | 9.7 ± 1.8 | 56.0 ± 5.6 |
| YETN | 7.83 ± 0.06 | 60.7 ± 0.7 | 5.92 ± 0.05 | 7.57 ± 0.30 | 128 ± 5 | 3.24 ± 0.07 | 2.73 ± 0.02 | 8.43 ± 0.20 | 9.8 ± 1.2 | 55.1 ± 6.1 |
| YTQN | 7.87 ± 0.06 | 63.1 ± 0.1 | 3.45 ± 0.29 | 9.60 ± 0.01 | 278 ± 23 | 2.55 ± 0.24 | 1.05 ± 0.63 | 4.11 ± 2.51 | 10.6 ± 1.2 | 49.2 ± 5.6 |
| YTTF | 8.56 ± 0.09 | 62.2 ± 0.1 | 12.7 ± 0.7 | 2.30 ± 0.01 | 18.0 ± 1.0 | 4.03 ± 0.06 | 0.12 ± 0.02 | 0.29 ± 0.04 | 43.8 ± 5.8 | 79.3 ± 9.9 |
| YTTY | 8.95 ± 0.02 | 62.0 ± 0.1 | 20.6 ± 0.6 | 1.71 ± 0.02 | 8.32 ± 0.2 | 5.40 ± 0.07 | 0.14 ± 0.04 | 0.26 ± 0.08 | 54.1 ± 7.1 | 67.9 ± 7.9 |
| YTWN | 8.14 ± 0.02 | 59.3 ± 0.2 | 4.83 ± 0.20 | 5.70 ± 0.03 | 118 ± 5 | 2.08 ± 0.07 | 1.09 ± 0.03 | 5.21 ± 0.22 | 15.9 ± 1.7 | 66.1 ± 7.1 |

A vast majority of the lead variants for both human and rat FcRn (FIGS. 5A and 5B, solid lines in various shades) had significantly slower on-rates than the WT or the benchmark variants (>2-fold) (Table 1). The N434F and N434Y mutations were the only variants which displayed an enhanced on-rate for both species of FcRn. Without being bound to any theory, as a result of the slower association kinetics with hFcRn, the apparent binding affinities of the lead variants were generally weaker than WT, unlike rFcRn (FIGS. 5C and 5D, Table 1). The affinities for rFcRn were weaker than YTE (FIG. 5D, diagonal lines facing bottom left, Table 2A). Without being bound to any theory, these results indicated that a single mutation was not sufficient to enhance the affinity to surpass the LS and YTE variants. Ranking the FcRn off-rates (due to the weak binding affinities of the variants to hFcRn) revealed a subset with reduced off-rates to both human and rat FcRn: M252Y, N434F/P/Y, T256D/E and T307A/E/F/Q/W (Table 2A). These variants are of further interest in combination to further improve the FcRn binding capabilities of the Fc region to surpass the benchmark variants.

In vitro characterization parameters for the lead variants are shown in Table 2B.

TABLE 2B
In vitro Characterization Parameters of Lead Variants.

| | | | Biacore | | | | | | |
|---|---|---|---|---|---|---|---|---|---|
| | | | FcγRIIIa | | | | | Biacore pH 7.4 | |
| | FcRn | | V158 | | | | | hFcRn | rFcRn |
| | Affinity | | Affinity | Biacore pH 6.0 | | | | Steady | Steady |
| mAb1 | Column | DSF | Fold | hFcRn | | | rFcRn | State | State |
| Variant | pH | Tₘ (° C.) | Change | On Rate | Off-rate | K_{D,app} | K_{D,app} | RU | RU |
| WT | 7.37 | 69.0 ± 0.2 | 1.00 | 16.2 ± 2.9 | 3.9 ± 0.4 | 2380 ± 470 | 207 ± 43 | 4.2 ± 0.9 | 13.0 ± 3.2 |
| LS | 8.29 | 68.5 ± 0.3 | 1.04 ± 0.04 | 1.9 ± 0.4 | 5.0 ± 0.1 | 272 ± 40 | 74 ± 9 | 18.3 ± 4.6 | 24.8 ± 4.8 |
| YTE | 8.14 | 61.2 ± 0.3 | 0.52 ± 0.03 | 1.4 ± 0.4 | 4.7 ± 0.1 | 342 ± 117 | 18 ± 2 | 13.2 ± 3.5 | 53.9 ± 1.2 |
| M252Y | 7.88 | 64.4 ± 0.2 | 0.46 ± 0.03 | 5.5 ± 1.8 | 1.4 ± 0.2 | >3000 | 25 ± 3 | 8.6 ± 1.0 | 50.5 ± 5.6 |
| N434F | 8.30 | 67.8 ± 0.2 | 1.16 ± 0.08 | 35 ± 15 | 0.5 ± 0.1 | 165 ± 73 | 26 ± 13 | 22.2 ± 2.6 | 61.5 ± 6.5 |
| N434Y | 8.46 | 67.3 ± 0.5 | 1.41 ± 0.06 | 36 ± 10 | 0.5 ± 0.1 | 137 ± 33 | 23 ± 12 | 25.5 ± 2.9 | 66.0 ± 7.1 |
| T256D | 7.82 | 64.7 ± 0.2 | 0.92 ± 0.04 | 4.4 ± 1.7 | 2.5 ± 0.7 | >3000 | 86 ± 27 | 5.8 ± 0.5 | 23.2 ± 2.8 |
| T256E | 7.63 | 66.3 ± 0.6 | 0.89 ± 0.04 | 3.9 ± 2.4 | 3.4 ± 0.2 | >3000 | 113 ± 15 | 4.5 ± 0.4 | 17.0 ± 2.1 |
| T307Q | 7.86 | 70.3 ± 0.6 | 1.00 ± 0.04 | 4.0 ± 1.1 | 2.2 ± 0.2 | >3000 | 58 ± 16 | 6.6 ± 0.7 | 31.3 ± 3.7 |
| T307W | 7.75 | 63.0 ± 0.5 | 0.97 ± 0.04 | 3.3 ± 0.8 | 2.8 ± 0.3 | >3000 | 111 ± 32 | 5.6 ± 0.7 | 21.9 ± 2.8 |
| DQ | 7.92 | 67.9 ± 0.4 | 0.73 ± 0.03 | 3.8 ± 0.4 | 8.7 ± 0.1 | 232 ± 24 | 9.5 ± 1.7 | 10.7 ± 1.0 | 39.1 ± 4.5 |
| DW | 7.92 | 57.8 ± 0.4 | 0.89 ± 0.04 | 5.3 ± 0.1 | 8.9 ± 0.3 | 169 ± 8 | 8.2 ± 0.9 | 12.2 ± 1.3 | 42.1 ± 4.7 |
| YD | 8.29 | 59.6 ± 0.9 | 0.48 ± 0.02 | 6.3 ± 1.2 | 5.9 ± 0.20 | 94 ± 18 | 5.9 ± 0.6 | 9.7 ± 1.8 | 56.0 ± 5.6 |

In Table 2B, all data was obtained using the experimental techniques at the top of each column. FcRn affinity chromatography, DSF and FcγRIIIa binding were performed in triplicate (n=3). FcRn binding kinetics to human and rat FcRn were obtained in quadruplicate and fit independently. Units: DSF Tₘ (° C.); FcγRIIIa Binding (Fold change relative to WT), Biacore pH 6.0 hFcRn On Rate (×10⁴ M⁻¹ s⁻¹), Off-rate (×10⁻¹ s⁻¹) and K_{D,app} (×10⁹ M); Biacore pH 6.0 rFcRn K_{D,app} (×10⁹ M); Biacore pH 7.4 hFcRn and rFcRn Steady State RU (RU).

### Example 4: The Combination Variants Further Decrease FcRn Binding Off-Rate

Multiple lead mutations were located at a single position (FIG. 14, black rectangles), such as T307 and N434, where six and three mutations, respectively, were identified that showed slower FcRn dissociation kinetics. Only mutations with the slowest FcRn off-rates to hFcRn at these positions were used for the creation of combination variants. In this case, T307Q, T307W, N434F and N434Y were mixed with M252Y, T256D and T256E to obtain double, triple and quadruple variants using mixed primer PCR and site directed mutagenesis. In total, the combination library consisted of 54 variants including the seven lead single, 18 double, 20 triple, 8 quadruple variants and the WT antibody. The nomenclature of these variants is as follows: the wild-type background contains M252, T256, T307 and N434 and is relabeled as MTTN. As such, the triple variant, YTQY, contains the M252Y, T307Q and N434Y mutations, while maintaining the WT threonine at position 256.

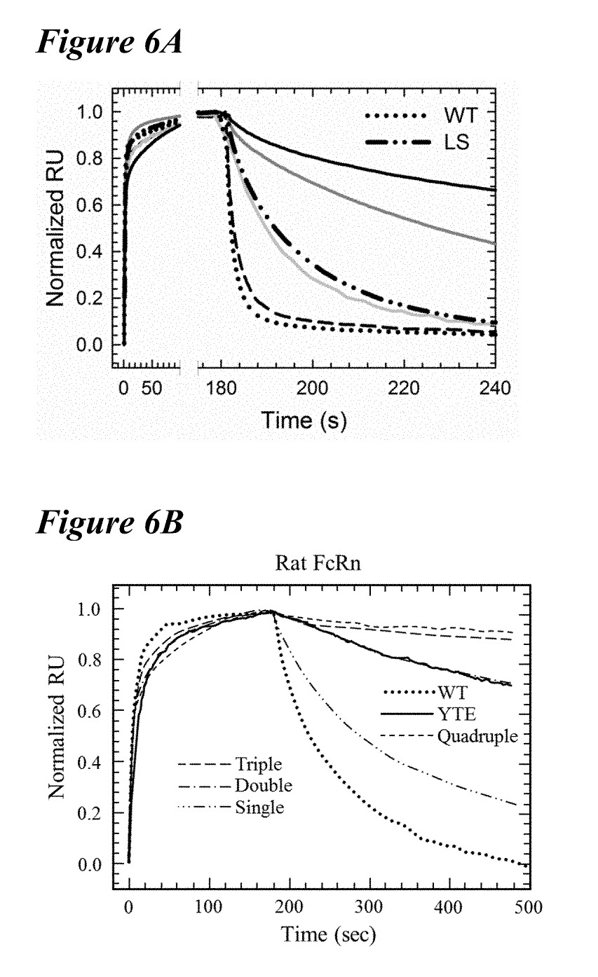

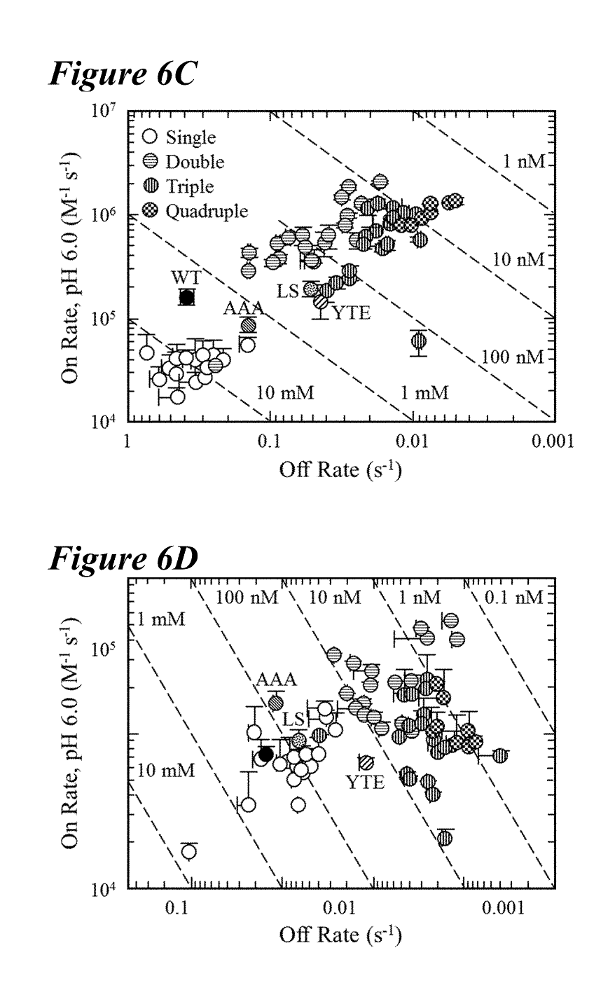

As with the single mutations, FcRn binding kinetics at pH 6.0 using Biacore was used to determine which combination variants have improved affinity. A representative FcRn binding kinetic trace of each the single (long dashes interspersed with two dots), double (long dashes interspersed with single dot), triple (long dashes) and quadruple (short dashes) are shown in FIGS. 6A and 6B, in comparison to the WT (dotted line) and the benchmark variant with the tightest affinity for their respective species of FcRn (hFcRn: LS (long dashes interspersed by two dots); rFcRn: YTE (solid line)). The hFcRn on and off-rates (FIG. 6C) revealed that two single, 15 double, 18 triple and eight quadruple variants had an enhanced binding affinity than the LS variant (FIG. 6C, dotted). Similarly, all combinations, except one triple variant, had a tighter affinity to rFcRn than the YTE (FIG. 6D, diagonal lines facing bottom left). In the case of hFcRn, additional FcRn-enhancing mutations further increased the binding affinity (FIG. 6C). The five combinations with the tightest affinity to hFcRn were all quadruple variants (FIG. 6C, checkered) with binding affinities approximately 500-fold greater than wild-type. A similar phenomenon did not occur with rFcRn (FIG. 6D), as the variants with the highest affinity were double variants (FIG. 6D, horizontal lines). The triple (FIG. 6D, vertical lines) and quadruple (FIG. 6D, checkered) variants typically showed only slight decreases in the off-rate (less than 2-fold), but also displayed decreased association-rates (FIG. 6D). Without being bound to any theory, these results suggest that a lower limit possibly exists regarding the FcRn apparent binding affinity (approximately 0.5 nM) that has been reached with rFcRn, but not hFcRn (FIG. 6B). In total, more than 40 combination variants had a tighter affinity than the benchmark variants and further characterization is required to select combinations with favorable properties for in vivo studies.

### Example 5: Combination Variants Retain Significant Binding at Physiological pH

As a result of the significantly improved FcRn affinity at pH 6.0, the effect on the pH-dependence was investigated using FcRn affinity chromatography and Biacore steady state measurements at pH 7.4. FcRn affinity chromatography employs a linear pH gradient to directly measure the perturbation of the pH-dependence by the mutations. H435A and H310A/H435Q, variants with weak FcRn binding, did not bind to the column regardless of pH (FIG. 8A). WT eluted near physiological pH (pH 7.37±0.05), while AAA, LS and YTE required a higher pH (Table 2B). All combination variants and the seven lead single variants required a higher pH to elute from the affinity column than WT (FIGS. 8A and 8C). The N434F/Y variants eluted at a greater pH than LS (Table 2B) which, without intending to be bound by scientific theory, indicates that these variants, both alone and in combination, disrupted the pH-dependence.

Representative chromatograms showed a clear shift to higher elution pH with the number of mutations (FIGS. 9A and 9B). A strong correlation (R2=0.94) between the elution pH and the hFcRn off-rates (FIG. 9C) indicates that the slower FcRn off-rate at pH 6.0 directly contributed to the increased elution pH for the FcRn variants.

FcRn binding kinetic experiments were performed at pH 7.4 using Biacore to measure the residual binding activity under physiological conditions. The steady state RU was used as a measure of residual FcRn binding affinity, as some variants showed unreliable kinetics and little to no binding at this pH. Representative kinetic traces of the single (long dashes interspersed with two dots), double (long dashes interspersed with single dot), triple (long dashes) and quadruple (short dashes) variants are shown in FIGS. 7A and 7B, in comparison to LS (FIG. 7A, solid) and YTE (FIG. 7B, solid). These two variants displayed the greatest residual binding to human and rat FcRn at pH 7.4, respectively. A majority of the lead single variants had slightly elevated FcRn binding in comparison to WT (4.3±1.0 RU), but less than AAA (13.1±1.7 RU), LS (18.5±2.6 RU) and YTE (13.1±1.6 RU), except for the N434F/Y mutations (Tables 2A and 2B). The combination variants also possessed significant residual binding to both species of FcRn at pH 7.4 (FIGS. 7A and 7B) to an even greater extent than N434F/Y. Without being bound to any theory, an ideal candidate for in vivo studies are variants with increased FcRn binding at low pH (such as the AAA, LS and YTE variants), but maintain a low level of binding at elevated pH, in a similar manner as the WT. In a plot shown in FIGS. 7C and 7D, these combinations would occupy the lower left quadrant designated by the affinities of the LS and YTE variants at each pH to human and rat FcRn, respectively.

### Example 6: FcRn Affinity Chromatography

The combination variants displayed a moderate positive correlation (hFcRn: R²=0.69, rFcRn: R²=0.71) between the apparent binding affinity at pH 6.0 and the steady state RU at pH 7.4 (FIGS. 7C and 7D). Without being bound by any theory, these results indicate that a higher affinity at pH 6.0 typically translates to a greater residual FcRn binding at pH 7.4. These variants could remain bound to FcRn in the bloodstream and have short serum half-lives and/or promote their clearance, similar to the high FcRn affinity abdeg mutations (see, e.g., Swiercz et al. 2014 supra; and Vaccaro et al. 2005 supra). As the antibody-FcRn interaction is pH-dependent and occurs only at low pH (<pH 6.5), the saturation mutations may strengthen the interaction through hydrophobic or charge-derived contributions, that may disrupt the deprotonation of the critical histidine residues (FIG. 1B, as indicated) and weakening of this interaction at physiological pH.

FcRn affinity chromatography employs a linear pH gradient to directly measure the perturbation of the FcRn interactions pH dependence (see, e.g., Schlothauer et al. 2013 supra). FcRn affinity chromatography with the AAA, LS, YTE, H435A and H310A/H435Q variants revealed that H435A (FIG. 8A, solid light gray line) and H310A/H435Q (FIG. 8A, AQ, solid dark gray line) do not bind to FcRn even at pH 5.5 and elute in the flow-through. The wild-type antibody eluted near physiological pH (pH 7.37±0.05), while AAA, LS and YTE, which have slower off-rates and tighter FcRn binding affinities than the wild-type by Octet (FIG. 2) and Biacore (FIG. 3), required a considerably higher pH to dissociate from the column (AAA: 7.94±0.06; LS: 8.29±0.03; YTE: 8.14±0.03). Elution profiles revealed that all of the variants in the combination library required a higher pH to elute from the affinity column than wild-type.

Representative chromatograms at the average elution pH for the single (long dashes interspersed with two dots), double (long dashes interspersed by single dot), triple (long dashes) and quadruple (short dashes) variants are shown in FIG. 9A. The seven lead single variants required a higher pH to dissociate from the column compared to WT (FIG. 10A, Table 3), while those with wild-type-like kinetics to hFcRn (K288D/N, Y436H/H/VV) all eluted at a similar pH to the wild-type.

TABLE 3
In vitro Characterization Parameters of Lead Antibody Variants

| | | | Biacore pH 6.0 | | | | | |
|---|---|---|---|---|---|---|---|---|
| | FcRn | | hFcRn | | | rFcRn | | |
| | Affinity | | On-rate | | | On-rate | | |
| | Column | DSF | (×10⁴ M⁻¹ | Off-rate | K_{D,app} | (×10⁴ M⁻¹ | Off-rate | K_{D,app} |
| Variant | pH | Tₘ (° C.) | s⁻¹) | (×10⁻¹ s⁻¹) | (×10⁹M) | s⁻¹) | (×10⁻³ s⁻¹) | (×10⁹M) |
| WT | 7.37 | 69.0 ± 0.2 | 16.2 ± 2.9 | 3.9 ± 0.4 | 2380 ± 470 | 7.3 ± 1.0 | 15.2 ± 0.2 | 207 ± 43 |
| E380C | 7.18 | 64.7 ± 0.5 | 1.7 ± 0.4 | 4.6 ± 1.5 | >10,000 | 1.7 ± 0.25 | 106 ± 1 | 6310 ± 880 |
| K288D | 7.33 | 65.8 ± 0.1 | 3.3 ± 1.1 | 5.1 ± 0.8 | >10,000 | 6.6 ± 3.0 | 8.4 ± 0.6 | 149 ± 63 |
| K288N | 7.39 | 66.7 ± 0.3 | 4.1 ± 1.4 | 4.5 ± 0.3 | >10,000 | 6.4 ± 2.8 | 10.7 ± 0.9 | 190 ± 73 |
| M252Y | 7.88 | 64.4 ± 0.2 | 5.5 ± 1.8 | 1.4 ± 0.2 | 3100 ± 1500 | 10.6 ± 3.1 | 2.6 ± 0.6 | 25 ± 3 |
| N434F | 8.30 | 67.8 ± 0.2 | 35 ± 15 | 0.5 ± 0.1 | 165 ± 73 | 12.6 ± 1.4 | 3.4 ± 1.8 | 26 ± 13 |
| N434P | 7.56 | 63.6 ± 0.5 | 2.4 ± 0.5 | 3.4 ± 1.1 | >10,000 | 3.4 ± 0.1 | 6.7 ± 0.2 | 194 ± 9 |
| N434Y | 8.46 | 67.3 ± 0.5 | 36 ± 10 | 0.5 ± 0.1 | 137 ± 33 | 14.5 ± 1.8 | 3.5 ± 1.9 | 23 ± 12 |
| T256D | 7.82 | 64.7 ± 0.2 | 4.4 ± 1.7 | 2.5 ± 0.7 | 6700 ± 3540 | 6.1 ± 1.8 | 4.8 ± 0.1 | 86 ± 27 |
| T256E | 7.63 | 66.3 ± 0.6 | 3.9 ± 2.4 | 3.4 ± 0.2 | >10,000 | 6.3 ± 1.6 | 6.9 ± 1.0 | 113 ± 15 |
| T307A | 7.61 | 68.0 ± 0.4 | 2.9 ± 0.7 | 2.9 ± 0.5 | >10,000 | 6.1 ± 2.7 | 7.1 ± 0.7 | 132 ± 48 |
| T307E | 7.58 | 70.2 ± 0.5 | 4.4 ± 1.6 | 3.0 ± 0.4 | 8130 ± 5070 | 5.6 ± 2.8 | 6.0 ± 0.2 | 141 ± 65 |
| T307F | 7.61 | 70.2 ± 0.3 | 2.7 ± 0.8 | 2.9 ± 0.1 | >10,000 | 5.7 ± 2.8 | 6.2 ± 0.1 | 131 ± 63 |
| T307M | 7.40 | 70.0 ± 0.4 | 4.1 ± 0.8 | 3.9 ± 0.3 | >10,000 | 6.8 ± 2.3 | 17.1 ± 4.0 | 279 ± 140 |
| T307Q | 7.86 | 70.3 ± 0.6 | 4.0 ± 1.1 | 2.2 ± 0.2 | 5720 ± 1530 | 7.4 ± 2.2 | 4.0 ± 0.3 | 58 ± 16 |
| T307W | 7.75 | 63.0 ± 0.5 | 3.3 ± 0.8 | 2.8 ± 0.3 | 8740 ± 2440 | 7.1 ± 2.3 | 7.4 ± 0.4 | 111 ± 32 |
| Y436H | 7.33 | 68.7 ± 0.3 | 2.6 ± 0.8 | 6.1 ± 1.0 | >10,000 | 5.1 ± 0.1 | 7.3 ± 0.9 | 131 ± 9 |
| Y436N | 7.22 | 65.8 ± 0.5 | 4.6 ± 2.4 | 7.4 ± 3.3 | >10,000 | 10.1 ± 4.9 | 20.3 ± 3.1 | 233 ± 86 |
| Y436W | 7.39 | 68.6 ± 0.7 | 2.8 ± 1.9 | 4.6 ± 0.9 | >10,000 | 3.4 ± 2.2 | 23.7 ± 7.7 | 1140 ± 950 |

All data were obtained using the experimental techniques at the top of each column. The elution pH was determined by FcRn affinity chromatography in triplicate (n=3) and DSF probed the thermal stability in triplicate (n=3). FcRn binding kinetics to human and rat FcRn were obtained from Biacore with a series of antibody concentrations (n=4) and fit independently. Units for each measurement are as follows: Elution pH (unit-less); DSF Tₘ (° C.); Biacore pH 6.0 hFcRn On-rate (×10⁴ M⁻¹ s⁻¹), Off-rate (×10⁻¹ s⁻¹) and K_{D,app} (×10⁹ M); Biacore pH 6.0 rFcRn On-rate (×10⁴ M⁻¹ s⁻¹), Off-rate (×10⁻³ s⁻¹) and K_{D,app} (×10⁹ M).

The N434F/Y variants both eluted at a greater pH than the LS variant (N434F: 8.30±0.05; N434Y: 8.46±0.02) and showed considerable FcRn binding at pH 7.4 (Table 4). These results indicate that these variants alone can disrupt the pH dependence. In general, the average elution pH increased with an increasing the number of FcRn binding enhancing mutations (FIG. 9B). A strong correlation (R²=0.94) appeared with the elution pH in comparison to the hFcRn off-rates (FIG. 9C); without being bound to any theory, indicating that the disruption of the pH dependence of the interaction directly contributes to the slower FcRn off-rates observed for the combination library at pH 6.0.

### Example 7: Thermal Stability

Most proteins, including antibodies, with low thermodynamic stability have an increased propensity for misfolding and aggregation and would limit or hinder their activity, efficacy and potential as novel therapeutics. The thermal stability of each variant was determined using DSF and the reported melting temperature (Tₘ) was defined as the midpoint of the first transition in the Sypro Orange fluorescence intensity profile. In comparison to the WT with a T, of 69.0±0.2° C., the LS variant is WT-like (68.5±0.3° C.) and AAA and YTE are thermally destabilized by −8° C. (AAA: 61.3±0.6° C.; YTE: 61.2±0.3° C.) (FIGS. 8B, 9B, and 10B; and Tables 2B, 3 and 4). In comparison to WT and LS with a T, of 69.0±0.2° C., the AAA and YTE variants had lower thermal stabilities by −8° C. by DSF.

TABLE 4
In vitro Characterization Parameters of the Benchmark and Lead Combinations

| | FcRn | | Biacore pH 6.0 | | | | | | | | |
|---|---|---|---|---|---|---|---|---|---|---|---|
| | Af- | | hFcRn | | | rFcRn | | | Biacore pH 7.4 | | |
| | finity | | On- | | | On- | | | hFcRn | rFcRn | FcγRIIIa |
| | Col- | DSF | rate | Off- | | rate | Off- | | Steady | Steady | V158 |
| | umn | Tₘ | (×10⁴ | rate | K_{D,app} | (×10⁴ | rate | K_{D,app} | State | State | K_{D,app} |
| Variant | pH | (° C.) | M⁻¹ s⁻¹) | (×10^{-1 s}⁻¹) | (×10⁹M) | M⁻¹ s⁻¹) | (×10^{-3 s}⁻¹) | (×10⁹M) | RU | RU | (×10⁹M) |
| WT | 7.37 | 69.0 ± 0.2 | 16.2 ± 2.9 | 3.9 ± 0.4 | 2380 ± 470 | 7.3 ± 1.0 | 15.2 ± 0.2 | 207 ± 43 | 4.2 ± 0.9 | 13.0 ± 3.2 | 467 ± 99 |
| AAA | 7.94 | 61.3 ± 0.6 | 8.4 ± 1.8 | 1.4 ± 0.1 | 1780 ± 380 | 15.7 ± 3.3 | 11.7 ± 1.1 | 77 ± 18 | 13.9 ± 3.1 | 23.6 ± 4.9 | 450 ± 19 |
| LS | 8.29 | 68.5 ± 0.3 | 19.3 ± 3.5 | 0.5 ± 0.1 | 272 ± 40 | 9.1 ± 1.6 | 6.6 ± 0.4 | 74 ± 9 | 18.3 ± 4.6 | 24.8 ± 4.8 | 369 ± 19 |
| YTE | 8.14 | 61.2 ± 0.3 | 14.3 ± 4.4 | 0.5 ± 0.1 | 342 ± 117 | 6.5 ± 0.5 | 1.2 ± 0.2 | 18 ± 2 | 13.2 ± 3.5 | 53.9 ± 1.2 | 1040 ± 160 |
| MDQN | 7.92 | 67.9 ± 0.4 | 3.8 ± 0.4 | 8.7 ± 0.1 | 232 ± 24 | 1.3 ± 0.2 | 1.3 ± 0.1 | 9.5 ± 1.7 | 10.7 ± 1.0 | 39.1 ± 4.5 | 600 ± 4 |
| MDWN | 7.92 | 57.8 ± 0.4 | 5.3 ± 0.1 | 8.9 ± 0.3 | 169 ± 8 | 1.6 ± 0.1 | 1.3 ± 0.1 | 8.2 ± 0.9 | 12.2 ± 1.3 | 42.1 ± 4.7 | 512 ± 30 |
| YDTN | 8.29 | 59.6 ± 0.9 | 6.3 ± 1.2 | 5.9 ± 0.20 | 94 ± 18 | 2.9 ± 0.1 | 1.7 ± 0.2 | 5.9 ± 0.6 | 9.7 ± 1.8 | 56.0 ± 5.6 | 1060 ± 60 |
| YETN | 7.83 | 60.7 ± 0.7 | 5.9 ± 0.1 | 7.6 ± 0.3 | 128 ± 5 | 3.2 ± 0.1 | 2.7 ± 0.1 | 8.4 ± 0.2 | 9.8 ± 1.2 | 55.1 ± 6.1 | 878 ± 101 |
| YTWN | 8.14 | 59.3 ± 0.2 | 4.8 ± 0.2 | 5.7 ± 0.1 | 118 ± 5 | 2.1 ± 0.1 | 1.1 ± 0.1 | 5.2 ± 0.2 | 15.9 ± 1.7 | 66.1 ± 7.1 | 896 ± 53 |
| YDQN | 8.51 | 60.5 ± 0.1 | 2.5 ± 0.2 | 2.8 ± 0.1 | 115 ± 7 | 0.5 ± 0.1 | 0.3 ± 0.1 | 5.1 ± 0.3 | 19.8 ± 2.7 | 65.2 ± 6.6 | 1060 ± 50 |
| YEQN | 8.12 | 61.9 ± 0.8 | 1.9 ± 0.1 | 4.1 ± 0.1 | 218 ± 6 | 0.5 ± 0.1 | 0.4 ± 0.1 | 7.8 ± 0.4 | 15.2 ± 2.1 | 61.8 ± 6.4 | 1620 ± 210 |

The mutations introduced into the wild-type backbone are in bold and underlined. All data was obtained using the experimental techniques shown at the top of each column. The elution pH and Tₘ was determined in triplicate (n=3). FcRn binding kinetics to human and rat FcRn at pH 6.0 were obtained from Biacore (n=4) and fit independently. Steady state FcRn binding response at pH 7.4 was measured using Biacore at a single antibody concentration in triplicate. The FcγRIIIa binding affinity was determined from a series of antibody concentrations in duplicate using Biacore. Units for each measurement are as follows: Elution pH (unit-less); DSF Tₘ (° C.); Biacore pH 6.0 hFcRn On-rate (×10⁵ M⁻¹ s⁻¹), Off-rate (×10⁻² s⁻¹) and K_{D,app} (×10⁹ M); Biacore pH 6.0 rFcRn On-rate (×10⁵ M⁻¹ s⁻¹), Off-rate (×10⁻³ s⁻¹) and K_{D,app} (×10⁹ M); Biacore pH 7.4 Steady State Binding Response (RU) and FcγRIIIa K_{D,app} (×10⁹ M).

Twelve of 18 lead saturation variants had a reduced Tₘ compared to wild-type, and several of the T307 mutants (T307E/F/M/Q) showed a slight stabilization (Table 4). None of the seven single variants used for combinations (FIG. 10B and Table 4) were significantly destabilized relative to YTE (FIG. 8B and Table 5). The addition of double (FIG. 9D, horizontal lines), triple (FIG. 9D, vertical lines) and quadruple (FIG. 9D, checkered) variants led to a further decrease in overall thermal stability compared to the single variants (FIG. 9D, white circles). Multiple variants exhibited a Tₘ lower than AAA or YTE (61.2±0. ° C.) with >60% of these variants containing T307W. The quadruple variants (FIG. 9D, checkered) showed a distinct bimodal distribution of melting temperatures with combinations containing T307Q having approximately 6° C. more thermal stability than those possessing T307W (FIG. 9D).

### Example 8: The Fc Variants Alter the Binding Interaction with FcγRIIIa

Besides the interaction with FcRn, the Fc regions hinge and C_{H}2 domains are responsible for the interaction with other Fc receptors, including FcγRIIIa. As five of the seven single variants used for the construction of the combination saturation library are located within the C_{H}2 domain, the ability to interact with these receptors may be compromised relative to wild-type, despite their location far from the interaction interface. Using Biacore to measure the FcγRIIIa binding in a similar manner as the FcRn binding at pH 7.4 revealed that the YTE (FIG. 11A, dark gray) variant showed an approximately 50% reduction in binding response compared to the wild-type (FIG. 11A, black). Without being bound to any theory, the reduced FcγRIIIa binding for YTE is a result of the M252Y mutation (FIG. 11B, lowest white circle) as this variant alone has significantly decreased affinity for this receptor. The other single mutations did not share this reduced affinity (FIG. 11B, white circles) and N434F/Y variants alone enhanced the binding by 16-40%. These effects were transferred to most, but not all, of their corresponding combinations. For example, M252Y-containing combinations had between a 17 and 72% reduction in FcγRIIIa binding (Table 5).

TABLE 5
Concentrations of the Saturation Library Variants in Conditioned Media

| | Position | | | | | | | | | | |
|---|---|---|---|---|---|---|---|---|---|---|---|
| | M252 | I253 | S254 | T256 | K288 | T307 | K322 | E380 | L432 | N434 | Y436 |
| Mutant | Concentration (μg mL⁻¹) | | | | | | | | | | |
| A | 89.4 | 3.4 | 99.5 | 4.5 | 106 | <0.1 | 110 | 206 | 173 | 108 | 22.0 |
| C | <0.1 | 0.2 | 108 | <0.1 | <0.1 | <0.1 | 323 | <0.1 | 138 | 31.8 | <0.1 |
| D | 22.7 | <0.1 | <0.1 | <0.1 | <0.1 | <0.1 | 169 | 127 | 133 | <0.1 | 2.2 |
| E | <0.1 | 136 | 93.0 | 163 | <0.1 | 23.5 | 167 | WT | 2.7 | 65.9 | 8.9 |
| F | <0.1 | <0.1 | <0.1 | <0.1 | <0.1 | <0.1 | 154 | 127 | 118 | <0.1 | <0.1 |
| G | 46.7 | <0.1 | 172 | 106 | <0.1 | 4.9 | 124 | 146 | 107 | <0.1 | <0.1 |
| H | <0.1 | <0.1 | <0.1 | 207 | <0.1 | 7.0 | 229 | 103 | 124 | <0.1 | 173 |
| I | <0.1 | WT | <0.1 | 193 | 53.9 | <0.1 | 63.0 | 60.2 | 182 | 16.3 | <0.1 |
| K | 1.2 | 4.1 | <0.1 | 3.2 | WT | 88.8 | WT | 69.2 | 25.0 | <0.1 | 67.0 |
| L | <0.1 | <0.1 | 103 | 124 | <0.1 | <0.1 | 181 | 169 | WT | 77.7 | <0.1 |
| M | WT | 80.0 | 1.5 | 122 | <0.1 | <0.1 | 112 | 76.8 | 97.3 | 30.5 | 90.1 |
| N | <0.1 | <0.1 | 89.6 | 46.8 | <0.1 | 126 | 231 | 190 | 235 | WT | 18.9 |
| P | 0.7 | <0.1 | 18.5 | 2.7 | 92.6 | <0.1 | 1.3 | 175 | <0.1 | 120 | <0.1 |
| Q | 176 | 35.7 | 20.9 | 123 | 1.0 | 14.0 | 216 | 197 | 188 | 55.4 | 96.9 |
| R | 0.8 | <0.1 | <0.1 | 189 | <0.1 | 1.5 | 209 | 9.1 | 142 | 102 | 25.0 |
| S | <0.1 | 71.1 | WT | 141 | <0.1 | 88.8 | 99.3 | 153 | 106 | <0.1 | 3.0 |
| T | 19.7 | 12.5 | <0.1 | WT | 83.8 | WT | 218 | 176 | 114 | 75.2 | 34.1 |
| V | 63.6 | 89.9 | 67.1 | 150 | <0.1 | <0.1 | 88.9 | 239 | 114 | 68.0 | 18.0 |
| W | 2.8 | <0.1 | <0.1 | 2.9 | 4.9 | <0.1 | 66.8 | 171 | <0.1 | 21.6 | 128 |
| Y | 0.7 | <0.1 | 150 | 117 | <0.1 | 1.1 | 143 | 11.3 | 26.1 | <0.1 | WT |

One variant, MDQF (FIG. 11B, highest in triple variant category), showed a dramatic 140% increase in FcγRIIIa binding. Thus, the combination saturation library offered variants with a wide range of Fc receptor functionalities that could be leveraged to tailor therapeutic antibodies with particular effector functions.

FIG. 110 shows a box plot of the FcγRIIIa binding responses of the seven lead single variants compared to the WT and YTE variants.

### Example 9: Seven Lead Combinations Balance the pH-Dependence of the FcRn Interaction

Without being bound to any theory, candidate variants for further study in vivo occupied the lower left quadrant of the plots shown in FIGS. 7C and 7D. Seven variants satisfied these criteria for hFcRn and comprised five double and two triple combinations (MDQN, MDWN, YDTN, YETN, YTWN, YDQN and YEQN), and did not contain a mutation at the N434 position (Table 3). Each of these combinations eluted from the FcRn affinity column between AAA (pH 7.94±0.06) and LS (pH 8.29±0.03) with YDQN eluting at the highest pH of 8.51±0.14 (FIG. 12A, Table 5), indicating only a slight perturbation in the pH-dependence and greater residual binding at pH 7.4 (Table 2A). One of the variants (MDQN) possessed a wild-type-like thermal stability, and six had a similar or reduced T, compared to the YTE variant (FIG. 12B, Table 4). In an FcγRIIIa binding assay, five combination variants showed a similar reduction as YTE (Table 4). Further investigation with the single mutations revealed that M252Y significantly affected FcγRIIIa binding and, without being bound by any theory, translates this effect towards combinations with this mutation. The remaining six single mutations were WT-like or possessed slightly improved binding to this receptor.

Three combination variants were selected for further studies based on their FcRn binding properties, thermal stabilities and FcγRIIIa binding. DQ (T256D/T307Q), DW (T256D/T307W) and YD (M252Y/T256D) each provided optimal FcRn binding properties (Table 2B) as the LS variant (FIG. 12E). Each variant offers a diverse range of thermal stability and FcγRIIIa binding properties (FIGS. 12F and 12G, Table 2B) that provided a range of functionality. FIG. 12H is a plot of homogeneous bridging RF.

The enhancement in apparent binding affinity for both human and rat FcRn at pH 6.0 compared to the LS (FIG. 13A, thick long dashes) and YTE variants (FIG. 13B, thick long dashes), respectively, was a compromise between the on- and off-rates (FIGS. 13A and 13B, Table 4). Typically, combinations with faster off-rates also possessed faster on-rates and vice versa. This observation was maintained between human and rat FcRn (Table 4). Furthermore, all of these variants had a lower steady state response than the LS variant (FIG. 13C, thick long dashes) to hFcRn at pH 7.4. These results were not consistent with rFcRn, as the five M252Y-containing variants, YDTN, YETN, YTWN, YDQN and YEQN, had elevated FcRn binding at pH 7.4 (FIG. 13, Table 5) compared to YTE. The MDQN and MDWN variants were the only combinations that were cross-reactive between human and rat FcRn. Furthermore, these two variants did not perturb the interaction with FcγRIIIa to a similar extent as the M252Y-containing variants (FIGS. 12C and 12D and Table 5; MDQN: 600±4 nM; MDWN: 512±30 nM; WT: 467±99 nM). Thus, saturation and combination mutagenesis at key FcRn interaction positions has led to the identification of lead variants that balanced the pH-dependence of the interaction, maintained functionality with an Fc receptor, could enhance FcRn functionality in vivo, and could extend the serum half-life of therapeutic antibodies.

### Example 10: Rheumatoid Factor Binding Characteristics of Lead Combination Variants

The isoelectric point and RF binding of the lead variants was investigated, as these mutations may alter antibody surface charge and immunogenicity. More acidic antibodies have been thought to prolong antibody pharmacokinetics. Compared to the WT and LS controls, all three leads resulted in a ˜0.2 pH unit reduction in the pl, as a result of the T256D substitution. FcRn-enhancing mutations may simultaneously alter binding to host antibodies, such as rheumatoid factor (RF), due to overlapping interaction interfaces. A homogeneous bridging ELISA was adapted to measure the change in RF binding for the lead variants. Interestingly, LS and YTE showed completely opposite shifts in RF binding compared to WT (FIG. 12H). LS significantly increased the RF binding, while YTE showed a significant decrease (p<0.001). YD (p<0.001) and DW (p<0.01) also significantly reduced RF binding, while DQ produced a similar response as WT. Without being bound to any theory, these results indicate that DQ, DW and YD can provide an immunogenic advantage compared to LS. The YD, DW and DQ variants represent a range of key antibody characteristics that can be leveraged in conjunction with the improved FcRn binding properties over the benchmark YTE and LS variants.

### Example 11: Lead Combination Variants are Transferable to Other Antibodies

A new binding assay was developed using a CM5 sensor chip, as depicted in FIG. 15A. The binding assay includes a step to immobilize streptavidin on a CM5 sensor chip to capture biotinylated FcRn to about 30 RU, replenished as necessary. Antibody binding kinetics were measured at pH 6.0 and 7.4, and pH 8.5 for regeneration. FIGS. 15B and 15C show the direct immobilization of FcRn and streptavidin capture of biotinylated FcRn respectively, using the new binding assay.

FcRn binding of Antibody-2 at pH 6.0: With mouse FcRn, lead Antibody-2 variants demonstrate slower off-rates than the LS variant (dashes) and wild-type (black) (FIG. 16A). For human FcRn, the lead variants all have faster on-rates but similar off-rates as LS (dashes) (FIG. 16B).

FcRn binding of Antibody-2 at pH 7.4: All lead variants showed a reduced human FcRn binding at pH 7.4 compared to LS (dashes) (FIG. 17A). As with the Antibody-1 background, the DW (MDWN) and DQ (MDQN) variants also showed lower residual binding to mouse (rat) FcRn at pH 7.4 (FIG. 17B).

Lead variants maintained a higher binding affinity at pH 6.0 and a lower residual binding at pH 7.4 compared to LS (FIG. 18). Importantly, variants were found to be transferable between different IgG1 backgrounds with little effect on FcRn binding. As shown in FIG. 19, LS had a similar elution pH regardless of the background. WT, DQ and DW in the Antibody-2 background showed a higher elution pH than in the Antibody-1 background, possibly as a result of tighter binding at pH 6.0 in the Antibody-2 background.

Antibody-2 background variants all showed a slightly increased thermal stability as shown in FIG. 20.

As shown in FIG. 21, similar to the Antibody-1 background, YD (YDTN) showed a reduction in FcγRIIIa binding response (left) and affinity (right). DQ (light gray) and DW (dark gray) showed FcγRIIIa binding properties similar to WT (black) in the Antibody-2 background. The effect on FcγRIIIa binding for LS is consistent between Antibody-1 and Antibody-2.

Thus, the lead variants in the Antibody-2 background do not significantly affect the FcRn binding, pH dependence, thermal stability, or FcγRIIIa binding as compared to the same lead variants in the Antibody-1 background.

In one embodiment, DQ (T256D/T307Q), DW (T256D/T307W) and YD (M252Y/T256D) variants were incorporated into an additional IgG1 antibody and a recombinant Fc fragment: mAb2 recognizes a different antigen from mAb1, and Ab3 is an Fc fragment. In each case, the pH-dependent FcRn binding kinetics (FIG. 22) were highly similar in addition to the elution pH, thermal stability and FcγRIIIa binding affinities (Table 2B, and Table 6). Without being bound to any theory, these results indicate that the DQ, DW and YD variants conferred their improved FcRn binding properties to proteins consisting of an Fc domain.

TABLE 6
Concentrations of the Saturation Library Variants in Conditioned Media

| | | | | Biacore | | | | | | |
|---|---|---|---|---|---|---|---|---|---|---|
| | | | | FcγRIIIa | | | | Biacore pH 7.4 | | |
| | | FcRn | | V158 | | | | hFcRn | cFcRn | mFcRn |
| | | Affinity | | Affinity | Biacore pH 6.0 | | | Steady | Steady | Steady |
| | | Column | DSF | Fold | hFcRn | cFcRn | mFcRn | State | State | State |
| Ab | Variant | pH | Tₘ | Change | *K_{D,app} | *K_{D,app} | *K_{D,app} | RU | RU | RU |
| 2 | WT | 7.61 | 69.3 ± 0.1 | 1.0 | 678 ± 97 | 1440 ± 360 | 107 ± 9 | 1.3 ± 0.2 | 1.3 ± 0.3 | 92 ± 8 |
| 2 | LS | 8.32 | 69.0 ± 0.1 | 1.23 ± 0.02 | 113 ± 22 | 210 ± 43 | 20 ± 9 | 44 ± 4 | 38 ± 6 | 285 ± 8 |
| 2 | DQ | 8.06 | 69.3 ± 0.1 | 1.05 ± 0.01 | 97 ± 33 | 110 ± 42 | 24 ± 11 | 14 ± 1 | 11 ± 1 | 248 ± 5 |
| 2 | DW | 8.11 | 58.1 ± 0.1 | 1.15 ± 0.01 | 69 ± 25 | 99 ± 9 | 13 ± 5 | 18 ± 2 | 15 ± 1 | 257 ± 8 |
| 2 | YD | 8.25 | 60.5 ± 0.1 | 0.58 ± 0.01 | 99 ± 49 | 120 ± 6 | 10 ± 3 | 27 ± 2 | 22 ± 3 | 394 ± 11 |
| 3 | WT | 7.62 | 67.5 ± 0.2 | 1.0 | 717 ± 23 | | 61 ± 1 | 2.2 ± 0.3 | | 72 ± 2 |
| 3 | LS | 8.32 | 66.6 ± 0.2 | 1.11 ± 0.03 | 51 ± 2 | | 16 ± 2 | 32 ± 2 | | 103 ± 1 |
| 3 | DQ | 8.07 | 63.8 ± 0.2 | 0.87 ± 0.02 | 51 ± 1 | | 24 ± 1 | 19 ± 2 | | 89 ± 1 |
| 3 | DW | 8.12 | 57.1 ± 0.2 | 0.93 ± 0.02 | 39 ± 6 | | 23 ± 1 | 22 ± 2 | | 99 ± 1 |
| 3 | YD | 8.23 | 59.5 ± 0.1 | 0.73 ± 0.02 | 54 ± 1 | | 0.5 ± 0.2 | 28 ± 3 | | 125 ± 2 |

### Example 12: Lead Variants Extended the In Vivo Plasma Antibody Elimination Half-Life

The pharmacokinetics (PK) of the DQ, DW and YD variants were examined for their effect on antibody circulation half-life with cynomolgus monkeys and hFcRn transgenic mice (strain Tg32) (see, e.g., Avery et al. Mabs (2016) 8: 1064-1078) in comparison to WT and LS controls. FcRn binding studies with cynomolgus FcRn revealed similar binding affinities to hFcRn (FIGS. 23A-23B; Table 6). Each animal was intravenously injected with the WT, LS, DQ, DW or YD variants, and the antibody concentration was quantified through a mass spectrometry approach to determine the clearance rate and serum half-life in monkeys (FIG. 24A) and hFcRn transgenic mice (FIG. 24B). The clearance rates and serum half-lives were obtained from a non-compartmental model of the antibody concentration as a function of time. All three lead variants and LS showed a significantly reduced clearance rate compared to WT in both monkeys and mice (p<0.001). The plasma half-life of the WT antibody was 9.9±0.5 and 11.7 days for monkeys and mice, respectively. Furthermore, the LS benchmark and variants identified exhibited a significant increase of elimination half-life compared to wild type in both species (2.5- and 1.7-fold increase in monkey and mouse, respectively) (Table 7). DQ, DW and YD showed a similar prolongation of half-life compared to the LS benchmark (Table 7). The DQ, DW and YD mutations identified herein through saturation mutagenesis demonstrated significantly prolonged plasma half-life than their WT counterparts in both mouse and non-human primate animal models.

TABLE 7
Clearance Rates and Serum Half-Lives of the Benchmark and Lead Variants

| | Cynomolgus Monkey (n = 3) | | | | hFcRn Tg32 Mouse (n = 6) | | | |
|---|---|---|---|---|---|---|---|---|
| | Clearance | | | | Clearance | | | |
| | (mL day⁻¹ | t_{1/2} (days) | | | (mL day⁻¹ | t_{1/2} (days) | | |
| mAb2 | kg⁻¹) | | Fold vs. | Fold vs. | kg⁻¹) | | Fold vs. | Fold vs. |
| Variant | Mean ± SD | Mean ± SD | WT | LS | Mean | Mean | WT | LS |
| WT | 5.6 ± 0.5 | 9.9 ± 0.5 | 1.0 | 0.4 | 7.4 | 11.7 | 1.0 | 0.6 |
| LS | 2.1** | 22.5 ± 2.4 | 2.3 | 1.0 | 4.6 | 19.5 | 1.7 | 1.0 |
| DQ | 3.4* | 20.8 | 2.1 | 0.9 | 3.2 | 24.5 | 2.1 | 1.3 |
| DW | 2.5 ± 0.2 | 20.4 ± 0.9 | 2.1 | 0.9 | 3.5 | 20.1 | 1.7 | 1.0 |
| YD | 2.4 ± 0.3 | 23.5 ± 2.1 | 2.4 | 1.0 | 4.5 | 17.5 | 1.5 | 0.9 |

In Table 7, the clearance rate and plasma-half-lives were determined using mAb2. Each clearance rate and half-life was the average of n=3 for the cynomolgus monkey and a single evaluation from a pool of n=6 hFcRn transgenic mice. Fold vs. WT and Fold vs. LS shows the relative improvement in serum half-life compared to the WT and LS, respectively. *n=2 due to ADA formation, **n=2 due to partial subcutaneous route of administration

### Example 13: Combination Variants with Enhanced FcRn Binding at pH 6.0 and pH 7.4

Based on the Octet screening (BLI-based screen) as described in Example 2, various single, double, triple, and quadruple variants were generated and their binding to FcRn at pH 6.0 and pH 7.4 were assessed (Table 8).

TABLE 8
Binding Affinity (pH 6.0) and Steady State Binding (pH 7.4) of Variants

| | | | | Steady | Steady | | |
|---|---|---|---|---|---|---|---|
| | | | | State | State | | |
| | | | Binding | Binding, | Binding | Ratio (pH | Ratio |
| | | Binding | Affinity | pH 7.4 | Error, pH | 6.0/pH | (pH 6.0/pH |
| Variant | Type | Affinity (M⁻¹) | Error (M⁻¹) | (RU) | 7.4 (RU) | 7.4) | 7.4) |
| WT | Benchmark | 420000 | 83000 | 4.2 | 0.9 | 100000 | 1.00E−05 |
| (MTTN) | | | | | | | |
| AAA | Benchmark | 562000 | 120000 | 13.9 | 3.1 | 40432 | 2.47E−05 |
| LS | Benchmark | 3680000 | 541000 | 18.3 | 4.6 | 201093 | 4.97E−06 |
| YTE | Benchmark | 2920000 | 1000000 | 13.2 | 3.5 | 221212 | 4.52E−06 |
| MDQN | Double | 4310000 | 446000 | 10.7 | 1 | 402804 | 2.48E−06 |
| MDTF | Double | 33400000 | 2240000 | 29.2 | 4 | 1143836 | 8.74E−07 |
| MDTY | Double | 64100000 | 2050000 | 37.1 | 5 | 1727763 | 5.79E−07 |
| MDWN | Double | 5920000 | 280000 | 12.2 | 1.3 | 485246 | 2.06E−06 |
| MEQN | Double | 2000000 | 24100 | 5.8 | 0.6 | 344828 | 2.90E−06 |
| METF | Double | 12800000 | 3350000 | 23.3 | 3.2 | 549356 | 1.82E−06 |
| METY | Double | 16000000 | 4130000 | 26.8 | 3.7 | 597015 | 1.68E−06 |
| MEWN | Double | 3100000 | 259000 | 7.7 | 0.9 | 402597 | 2.48E−06 |
| MTQF | Double | 23300000 | 4290000 | 34.2 | 4.6 | 681287 | 1.47E−06 |
| MTQY | Double | 30900000 | 6950000 | 38.4 | 5.2 | 804688 | 1.24E−06 |
| MTWF | Double | 26200000 | 1230000 | 30.5 | 4.2 | 859016 | 1.16E−06 |
| MTWY | Double | 46500000 | 2600000 | 37.2 | 4.9 | 1250000 | 8.00E−07 |
| YDTN | Double | 10700000 | 2100000 | 9.7 | 1.8 | 1103093 | 9.07E−07 |
| YETN | Double | 7810000 | 305000 | 9.8 | 1.2 | 796939 | 1.25E−06 |
| YTQN | Double | 3600000 | 298000 | 10.6 | 1.2 | 339623 | 2.94E−06 |
| YTTF | Double | 55600000 | 3090000 | 43.8 | 5.8 | 1269406 | 7.88E−07 |
| YTTY | Double | 120000000 | 2890000 | 54.1 | 7.1 | 2218115 | 4.51E−07 |
| YTWN | Double | 8470000 | 359000 | 15.9 | 1.7 | 532704 | 1.88E−06 |
| MDQF | Triple | 6620000 | 1840000 | 55.3 | 11.2 | 119711 | 8.35E−06 |
| MDQY | Triple | 36900000 | 4360000 | 49.4 | 6.7 | 746964 | 1.34E−06 |
| MDWF | Triple | 28100000 | 2840000 | 47.1 | 6.4 | 596603 | 1.68E−06 |
| MDWY | Triple | 84000000 | 9890000 | 59 | 7.9 | 1423729 | 7.02E−07 |
| MEQF | Triple | 142000 | 6490 | 8.6 | 0.8 | 16512 | 6.06E−05 |
| MEQY | Triple | 23800000 | 2660000 | 38.6 | 5.2 | 616580 | 1.62E−06 |
| MEWF | Triple | 56200000 | 8520000 | 41.9 | 5.6 | 1341289 | 7.46E−07 |
| MEWY | Triple | 70400000 | 7440000 | 46.6 | 6.3 | 1510730 | 6.62E−07 |
| YDQN | Triple | 8700000 | 560000 | 19.8 | 2.7 | 439394 | 2.28E−06 |
| YDTF | Triple | 29600000 | 2540000 | 57.7 | 7.7 | 512998 | 1.95E−06 |
| YDTY | Triple | 90100000 | 812000 | 65.4 | 8.9 | 1377676 | 7.26E−07 |
| YDWN | Triple | 10100000 | 1540000 | 25.9 | 3.6 | 389961 | 2.56E−06 |
| YEQN | Triple | 4590000 | 126000 | 15.2 | 2.1 | 301974 | 3.31E−06 |
| YETY | Triple | 33400000 | 3580000 | 22.6 | 2.9 | 1477876 | 6.77E−07 |
| YEWN | Triple | 6410000 | 904000 | 69.6 | 9.3 | 92098 | 1.09E−05 |
| YTQF | Triple | 56500000 | 1280000 | 59.9 | 8 | 943239 | 1.06E−06 |
| YTQY | Triple | 63300000 | 4010000 | 71.5 | 9.6 | 885315 | 1.13E−06 |
| YTWF | Triple | 65400000 | 2990000 | 62.6 | 8.2 | 1044728 | 9.57E−07 |
| YTWY | Triple | 106000000 | 10600000 | 75.1 | 9.8 | 1411451 | 7.08E−07 |
| YDQF | Quadruple | 111000000 | 4320000 | 68.6 | 10 | 1618076 | 6.18E−07 |
| YDQY | Quadruple | 235000000 | 6060000 | 80.2 | 12 | 2930175 | 3.41E−07 |
| YDWF | Quadruple | 166000000 | 3050000 | 71.8 | 9.9 | 2311978 | 4.33E−07 |
| YDWY | Quadruple | 266000000 | 19100000 | 88.5 | 11.7 | 3005650 | 3.33E−07 |

In Table 8, binding affinity to FcRn at pH 6.0 and steady state binding to FcRn at pH 7.4 for various single, double, triple, and quadruple mutants, as well as benchmark variants (AAA, LS, YTE) are shown.

These values are plotted in FIG. 25. FIG. 25 shows a comparison of the binding affinity at pH 6.0 and the RU at pH 7.4. As shown, the benchmark variant LS has the tightest binding affinity at pH 6.0 and largest residual binding at pH 7.4 of the benchmark variants tested (AAA, LS, YTE).

It was determined that several combination variants shown in FIG. 25 exhibited enhanced FcRn binding affinity at pH 6.0 and at pH 7.4. To investigate whether any of the combination variants showed a tighter binding than the MST-HN variant (referred to herein as “the YTEKF benchmark,” containing mutations at Met252, Ser254, Thr256, His433 and Asn434 to Tyr252, Thr254, Glu256, Lys433 and Phe434) at both pH 6.0 and pH 7.4, the following methodology was performed. Capture of biotinylated human, cynomolgus and mouse FcRn was performed via the Biotin CAPture method (see FIG. 26 for schematic). For pH 6.0, a concentration series (5 pts) from 1000 nM was performed in duplicate. For pH 7.4, a single concentration (1000 nM) injection was performed in triplicate (capture level of each FcRn was increased 10-fold to observe binding at this pH). Association: 180 sec; Dissociation: 300 sec.

Human FcRn binding kinetics at pH 6.0 of the YTEKF benchmark and various combination variants are shown in FIG. 27. As shown in FIG. 27, All examined variants showed a two-order magnitude tighter affinity to human FcRn compared to wild type (WT).

FIGS. 28A and 28B show the FcRn binding kinetics of the combination variants in comparison to the YTEKF benchmark at pH 6.0 (FIG. 28A) and at pH 7.4 (FIG. 28B). In FIG. 28A, a majority of the variants exhibited slower off-rates than the YTEKF benchmark, and had similar or slower on-rates. In FIG. 28B, YTEKF exhibits significant binding at pH 7.4, and four variants show a higher residual binding.

TABLE 9
Binding Affinity (pH 6.0) and Steady State Binding (pH 7.4) of Select
Variants

| Human FcRn | |
|---|---|
| pH 6.0 | pH 7.4 |

| Variant | KD (nM) | SD | RU | SD |
|---|---|---|---|---|
| YDQY | 2.7 | 0.1 | 103.1 | 0.6 |
| YEWY | 8.3 | 0.5 | 98.2 | 0.9 |
| YEQY | 4.8 | 0.2 | 93.6 | 0.8 |
| YDQF | 4.7 | 0.1 | 91.7 | 0.6 |
| YDWY | 5.2 | 0.2 | 91.2 | 0.5 |
| YTEKF | 14.8 | 0.3 | 89.9 | 1.3 |
| YWY | 10.0 | 0.4 | 77.1 | 0.3 |
| YDWF | 11.0 | 0.4 | 75.7 | 0.5 |
| YDY | 15.1 | 0.8 | 69.7 | 0.4 |
| DWY | 18.1 | 1.1 | 59.8 | 0.8 |
| YY | 22.9 | 0.9 | 54.4 | 0.1 |
| WT | 1288 | 201 | 0.4 | 0.1 |

In Table 9, binding affinity to FcRn at pH 6.0 and steady state binding to FcRn at pH 7.4 for select combination variants, as well as the YTEKF benchmark and WT are shown.

FIG. 29 shows a comparison of the binding affinity at pH 6.0 and the RU at pH 7.4 for select combination variants as indicated in Table 9. As shown in Table 9 and FIG. 29, four quadruple variants were found to have a higher affinity at pH 6.0 and pH 7.4 for FcRn compared to the YTEKF benchmark. The four quadruple variants favored the T256D, T307Q, and N434Y mutations. These quadruple variants exhibited an approximately 500-fold and 3-fold improvement in affinity (at pH 6.0) over WT and YTEKF, respectively.

Other characterization parameters, e.g., thermal stability, binding to FcγRIIIa, and elution pH were determined and shown in Table 10.

TABLE 10
Other Characterization Parameters of Lead Quadruple Variants

| | Affinity, pH | Steady State | Tm | FcγRIIIa | Elution |
|---|---|---|---|---|---|
| Variant | 6.0 (nM) | RU, pH 7.4 | (° C.) | Binding (RU) | pH |
| YDQY | 2.7 | 103.1 | 59.0 | 119 | 9.2 |
| YEWY | 8.3 | 98.2 | 52.7 | 92 | 9.3 |
| YEQY | 4.8 | 93.6 | 59.5 | 108 | 9.1 |
| YDQF | 4.7 | 91.7 | 59.1 | 93.2 | 8.8 |
| YDWY | 5.2 | 91.2 | 52.5 | 104 | 9.5 |
| WT | ~1500 | <1 | 69.0 | 142 | 7.37 |
| YTEKF | 14.8 | 89.9 | N.P.* | N.P.* | N.P. |

*Without intending to be bound by theory, due to the presence of “YTE” in YTEKF, the thermal stability and FcγRIIIa binding are expected to be similar to the lead quadruple variants

As shown in Table 10, all lead quadruple variants were found to be thermally destabilized and exhibited reduced FcγRIIIa binding capabilities.

1. An isolated binding polypeptide comprising a modified Fc domain, comprising:
an aspartic acid (D) or a glutamic acid (E) at amino acid position 256, and/or a tryptophan (W) or a glutamine (Q) at amino acid position 307, wherein amino acid position 254 is not threonine (T), and further comprising:
a phenylalanine (F) or a tyrosine (Y) at amino acid position 434; or
a tyrosine (Y) at amino acid position 252,
wherein amino acid positions are according to EU numbering.

2. An isolated binding polypeptide comprising a modified Fc domain comprising a combination of amino acid substitutions at positions selected from the group consisting of:
a) a tyrosine (Y) at amino acid position 252, and an aspartic acid (D) at amino acid position 256;
b) an aspartic acid (D) at amino acid position 256, and a phenylalanine (F) at amino acid position 434;
c) an aspartic acid (D) at amino acid position 256, and a tyrosine (Y) at amino acid position 434;
d) a tryptophan (W) at amino acid position 307, and a phenylalanine (F) at amino acid position 434;
e) a tyrosine (Y) at amino acid position 252, and a tryptophan (W) at amino acid position 307, wherein a tyrosine (Y) is not at amino acid position 434;
f) an aspartic acid (D) at amino acid position 256, and a tryptophan (W) at amino acid position 307, wherein a tyrosine (Y) is not at amino acid position 434;
g) an aspartic acid (D) at amino acid position 256, and a glutamine (Q) at amino acid position 307, wherein a tyrosine (Y) is not at amino acid position 434;
h) a tyrosine (Y) at amino acid position 252, an aspartic acid (D) at amino acid position 256, and a glutamine (Q) at amino acid position 307, wherein a tyrosine (Y) is not at amino acid position 434; and
i) a tyrosine (Y) at amino acid position 252, a glutamic acid (E) at amino acid position 256, and a glutamine (Q) at amino acid position 307, wherein a threonine (T) is not at amino acid position 254, a histidine (H) is not at amino acid position 311, and a tyrosine (Y) is not at amino acid position 434;
wherein the amino acid substitutions are according to EU numbering.

3. An isolated binding polypeptide comprising a modified Fc domain comprising:
a) a double amino acid substitution selected from the group consisting of M252Y/T256D, M252Y/T256E, M252Y/T307Q, M252Y/T307W, T256D/T307Q, T256D/T307W, T256E/T307Q, and T256E/T307W, wherein a threonine (T) is not at amino acid position 254, a histidine (H) is not at amino acid position 311, and a tyrosine (Y) is not at amino acid position 434; or
b) a triple amino acid substitution selected from the group consisting of M252Y/T256D/T307Q, M252Y/T256D/T307W, M252Y/T256E/T307Q, and M252Y/T256E/T307W, wherein a threonine (T) is not at amino acid position 254, a histidine (H) is not at amino acid position 311, and a tyrosine (Y) is not at amino acid position 434;
wherein the amino acid substitutions are according to EU numbering.

4. The isolated binding polypeptide of claim 1, optionally wherein:
the modified Fc domain is a modified human Fc domain;
the modified Fc domain is a modified IgG1 Fc domain;
the binding polypeptide has human FcRn binding affinity;
the binding polypeptide has rat FcRn binding affinity;
the binding polypeptide has human and rat FcRn binding affinity;
the isolated binding polypeptide has an altered serum half-life compared to a binding polypeptide comprising a wild-type Fc domain, optionally wherein the isolated binding polypeptide has an increased serum half-life compared to a binding polypeptide comprising a wild-type Fc domain;
the isolated binding polypeptide has altered FcRn binding affinity compared to a binding polypeptide comprising a wild-type Fc domain, optionally wherein the isolated binding polypeptide has enhanced FcRn binding affinity compared to a binding polypeptide comprising a wild-type Fc domain, optionally wherein the enhanced FcRn binding affinity comprises a reduced FcRn binding off-rate;
the isolated binding polypeptide has enhanced FcRn binding affinity at an acidic pH compared to a binding polypeptide comprising a wild-type Fc domain, optionally wherein the enhanced FcRn binding affinity comprises a reduced FcRn binding off-rate; and/or
the isolated binding polypeptide has enhanced FcRn binding affinity at an acidic pH compared to the FcRn binding affinity of the binding polypeptide at an elevated non-acidic pH, optionally wherein the enhanced FcRn binding affinity comprises a reduced FcRn binding off-rate.

5-15. (canceled)

16. The isolated binding polypeptide of claim 4, optionally wherein:
the acidic pH is about 6.0; and/or
the acidic pH is about 6.0 and the non-acidic pH is about 7.4.

17. (canceled)

18. The isolated binding polypeptide of claim 1, optionally wherein:
the isolated binding polypeptide is an antibody;
the isolated binding polypeptide is a monoclonal antibody;
the isolated antibody is a chimeric, humanized, or human antibody;
the isolated antibody is a full-length antibody;
the isolated binding polypeptide specifically binds one or more human targets; or
the isolated binding polypeptide has altered FcγRIIIa binding affinity compared to a binding polypeptide comprising a wild-type Fc domain, optionally wherein:
the isolated binding polypeptide has reduced FcγRIIIa binding affinity compared
to a binding polypeptide comprising a wild-type Fc domain;
the isolated binding polypeptide has enhanced FcγRIIIa binding affinity compared to a binding polypeptide comprising a wild-type Fc domain;
the isolated binding polypeptide has approximately the same FcγRIIIa binding affinity as a binding polypeptide comprising a wild-type Fc domain;
the isolated binding polypeptide has approximately the same thermal stability as a binding polypeptide comprising a wild-type Fc domain; or
the isolated binding polypeptide has approximately the same thermal stability as a binding polypeptide comprising a modified Fc domain having the triple amino acid substitution M252Y/S254T/T256E, according to EU numbering.

19-28. (canceled)

29. An isolated nucleic acid molecule comprising a nucleic acid encoding the isolated polypeptide of claim 1.

30. A vector comprising the isolated nucleic acid molecule of claim 29, optionally wherein the vector is an expression vector.

31. (canceled)

32. A host cell comprising the vector of claim 30, optionally wherein:
the host cell is of eukaryotic or prokaryotic origin;
the host cell is of mammalian origin; and/or
the host cell is of bacterial origin.

33-35. (canceled)

36. A pharmaceutical composition comprising the isolated binding polypeptide of claim 1.

37. (canceled)

38. An isolated binding polypeptide comprising a modified Fc domain, wherein the modified Fc domain comprises:
an aspartic acid (D) at amino acid position 256, and a glutamine (Q) at amino acid position 307, according to EU numbering;
an aspartic acid (D) at amino acid position 256, and a tryptophan (W) at amino acid position 307, according to EU numbering; or
a tyrosine (Y) at amino acid position 252, and an aspartic acid (D) at amino acid position 256, according to EU numbering.

39-60. (canceled)

61. An isolated binding polypeptide comprising a modified Fc domain, wherein the modified Fc domain comprises a combination of at least four amino acid substitutions comprising:
an aspartic acid (D) or a glutamic acid (E) at amino acid position 256, and a tryptophan (W) or a glutamine (Q) at amino acid position 307, wherein amino acid position 254 is not threonine (T), and further comprising:
a phenylalanine (F) or a tyrosine (Y) at amino acid position 434; and
a tyrosine (Y) at amino acid position 252,
wherein amino acid positions are according to EU numbering; or
an isolated binding polypeptide comprising a modified Fc domain having a combination of amino acid substitutions at positions selected from the group consisting of:
a) a tyrosine (Y) at amino acid position 252, an aspartic acid (D) at amino acid position 256, a glutamine (Q) at amino acid position 307, and a tyrosine (Y) at amino acid position 434;
b) a tyrosine (Y) at amino acid position 252, a glutamic acid (E) at amino acid position 256, a tryptophan (W) at amino acid position 307, and a tyrosine (Y) at amino acid position 434;
c) a tyrosine (Y) at amino acid position 252, a glutamic acid (E) at amino acid position 256, a glutamine (Q) at amino acid position 307, and a tyrosine (Y) at amino acid position 434;
d) a tyrosine (Y) at amino acid position 252, an aspartic acid (D) at amino acid position 256, a glutamine (Q) at amino acid position 307, and a phenylalanine (F) at amino acid position 434: or
e) a tyrosine (Y) at amino acid position 252, an aspartic acid (D) at amino acid position 256, a tryptophan (W) at amino acid position 307, and a tyrosine (Y) at amino acid position 434,
wherein the amino acid substitutions are according to EU numbering; or
an isolated binding polypeptide comprising a modified Fc domain comprising:
a quadruple amino acid substitution selected from the group consisting of M252Y/T256D/T307Q/N434Y, M252Y/T256E/T307W/N434Y, M252Y/T256E/T307Q/N434Y, M252Y/T256D/T307Q/N434F, and M252Y/T256D/T307W/N434Y,
wherein the amino acid substitutions are according to EU numbering.

62. (canceled)

63. (canceled)

64. The isolated binding polypeptide of claim 61, optionally wherein:
the modified Fc domain is a modified human Fc domain;
the modified Fc domain is a modified IgG1 Fc domain;
the binding polypeptide has human FcRn binding affinity;
the binding polypeptide has rat FcRn binding affinity;
the binding polypeptide has human and rat FcRn binding affinity;
the isolated binding polypeptide has altered FcRn binding affinity compared to a binding polypeptide comprising a wild-type Fc domain;
the isolated binding polypeptide has enhanced FcRn binding affinity compared to a binding polypeptide comprising a wild-type Fc domain;
the isolated binding polypeptide has enhanced FcRn binding affinity at an acidic pH compared to a binding polypeptide comprising a wild-type Fc domain;
the isolated binding polypeptide has enhanced FcRn binding affinity at an acidic pH compared to a binding polypeptide comprising M252Y/S254T/T256E/H433K/N434F;
the isolated binding polypeptide has enhanced FcRn binding affinity at a non-acidic pH compared to a binding polypeptide comprising a wild-type Fc domain;
the isolated binding polypeptide has enhanced FcRn binding affinity at a non-acidic pH compared to a binding polypeptide comprising M252Y/S254T/T256E/H433K/N434F;
the isolated binding polypeptide has enhanced FcRn binding affinity at an acidic pH, and enhanced FcRn binding affinity at a non-acidic pH, compared to a binding polypeptide comprising a wild-type Fc domain; and/or
the isolated binding polypeptide has enhanced FcRn binding affinity at an acidic pH, and enhanced FcRn binding affinity at a non-acidic pH, compared to a binding polypeptide comprising M252Y/S254T/T256E/H433K/N434F.

65-76. (canceled)

77. The isolated binding polypeptide of claim 64, wherein the acidic pH is about 6.0, and/or the non-acidic pH is about 7.4.

78. (canceled)

79. The isolated binding polypeptide of claim 61, optionally wherein:
the isolated binding polypeptide has an altered serum half-life compared to a binding polypeptide comprising a wild-type Fc domain;
the isolated binding polypeptide has a reduced serum half-life compared to a binding polypeptide comprising a wild-type Fc domain;
the isolated binding polypeptide has a reduced serum half-life compared to a binding polypeptide comprising M252Y/S254T/T256E/H433K/N434F;
the isolated binding polypeptide has altered FcγRIIIa binding affinity compared to a binding polypeptide comprising a wild-type Fc domain;
the isolated binding polypeptide has reduced FcγRIIIa binding affinity compared to a binding polypeptide comprising a wild-type Fc domain;
the isolated binding polypeptide has reduced FcγRIIIa binding affinity compared to a binding polypeptide comprising M252Y/S254T/T256E/H433K/N434F;
the isolated binding polypeptide has reduced thermal stability compared to a binding polypeptide comprising a wild-type Fc domain;
the isolated binding polypeptide has reduced thermal stability compared to a binding polypeptide comprising M252Y/S254T/T256E/H433K/N434F;
the isolated binding polypeptide is an antibody;
the isolated binding polypeptide is a monoclonal antibody;
the isolated antibody is a chimeric, humanized, or human antibody;
the isolated antibody is a full-length antibody; and/or
the isolated binding polypeptide specifically binds one or more targets.

80-91. (canceled)

92. An isolated nucleic acid molecule comprising a nucleic acid encoding the isolated polypeptide of claim 61.

93. A vector comprising the isolated nucleic acid molecule of claim 92, optionally wherein the vector is an expression vector.

94. (canceled)

95. A host cell comprising the vector of claim 93, optionally wherein:
the host cell is of eukaryotic or prokaryotic origin;
the host cell is of mammalian origin; and/or
the host cell is of bacterial origin.

96-98. (canceled)

99. A pharmaceutical composition comprising the isolated binding polypeptide of claim 61.

100. A pharmaceutical composition comprising the isolated antibody of claim 79.

101. An isolated binding polypeptide comprising a modified Fc domain comprising:
a tyrosine (Y) at amino acid position 252, an aspartic acid (D) at amino acid position 256, a glutamine (Q) at amino acid position 307, and a tyrosine (Y) at amino acid position 434, according to EU numbering;
a tyrosine (Y) at amino acid position 252, a glutamic acid (E) at amino acid position 256, a tryptophan (W) at amino acid position 307, and a tyrosine (Y) at amino acid position 434, according to EU numbering;
a tyrosine (Y) at amino acid position 252, a glutamic acid (E) at amino acid position 256, a glutamine (Q) at amino acid position 307, and a tyrosine (Y) at amino acid position 434, according to EU numbering;
a tyrosine (Y) at amino acid position 252, an aspartic acid (D) at amino acid position 256, a glutamine (Q) at amino acid position 307, and a phenylalanine (F) at amino acid position 434, according to EU numbering; or
a tyrosine (Y) at amino acid position 252, an aspartic acid (D) at amino acid position 256, a tryptophan (W) at amino acid position 307, and a tyrosine (Y) at amino acid position 434, according to EU numbering.

102-124. (canceled)

125. A method of treating a disease or disorder in a subject in need thereof, comprising administering to the subject a therapeutically effective amount of the isolated binding polypeptide of claim 1, optionally wherein the disease or disorder is a cancer, optionally wherein the cancer is a tumor, or optionally wherein the disease or disorder is an autoimmune disorder.

126-128. (canceled)

129. A method of treating a cancer in a subject in need thereof, comprising administering to the subject a therapeutically effective amount of the isolated binding polypeptide of claim 1.

130. (canceled)

131. A method of treating a cancer in a subject in need thereof, comprising administering to the subject a therapeutically effective amount of the isolated binding polypeptide of claim 61.
